(19) United States
(12) Patent Application Publication
(10) Pub. No.: US 2024/0343831 A1
(43) Pub. Date: Oct. 17, 2024

(54) ANTI-TMPRSS2 ANTIBODIES AND ANTIGEN-BINDING FRAGMENTS

(71) Applicant: Regeneron Pharmaceuticals, Inc., Tarrytown, NY (US)

(72) Inventors: Alina BAUM, Pleasantville, NY (US); Christos KYRATSOUS, Irvington, NY (US); Lisa PURCELL, Yorktown Heights, NY (US)

(21) Appl. No.: 18/642,721

(22) Filed: Apr. 22, 2024

Related U.S. Application Data
(63) Continuation of application No. 17/171,941, filed on Feb. 9, 2021, now Pat. No. 11,999,799.
(60) Provisional application No. 62/972,338, filed on Feb. 10, 2020.
(60) Provisional application No. 63/021,016, filed on May 6, 2020.
(60) Provisional application No. 63/094,199, filed on Oct. 20, 2020.

(51) Int. Cl. C07K 16/40; A61K 39/395; A61K 45/06; C12N 9/50

(52) U.S. Cl. CPC C07K 16/40; A61K 39/3955; A61K 45/06; C12N 9/50; C07K 2317/31; C07K 2317/33; C07K 2317/92

(57) ABSTRACT

The present disclosure provides antibodies and antigen-binding fragments thereof that bind specifically to TMPRSS2 and methods of using such antibodies and fragments for treating or preventing viral infections (e.g., coronavirus infections or influenza virus infections).

### RELATED APPLICATIONS

This application is a continuation of U.S. patent application Ser. No. 17/171,941, filed Feb. 9, 2021, which claims priority under 35 U.S.C. 119 (e) to U.S. Provisional Patent Application Ser. No. 62/972,338, filed Feb. 10, 2020; U.S. Provisional Patent Application Ser. No. 63/021,016, filed May 6, 2020; and U.S. Provisional Patent Application Ser. No. 63/094,199, filed Oct. 20, 2020, the disclosure of which are hereby incorporated by reference in their entirety.

### GOVERNMENT LICENSE RIGHTS

This invention was made with Government support under Agreement HHSO100201700020C, awarded by the U.S. Department of Health and Human Services. The Government has certain rights in the invention.

### SEQUENCE LISTING

A copy of the sequence listing is submitted concurrently with the specification electronically via Patent Center. The content of the electronic sequence listing (10737US02_Sequence Listing_ST26.xml; Size 90,112 bytes; and Date of Creation: Apr. 22, 2024) is herein incorporated by reference in its entirety.

### FIELD OF THE INVENTION

The present invention relates to antibodies and antigen-binding fragments that bind specifically to TMPRSS2 and methods for treating or preventing viral infections with said antibodies and fragments.

### BACKGROUND OF THE INVENTION

Viruses such as influenza virus have acquired resistance to currently used drugs that target the viral neuraminidase (NA) or the ion channel protein, matrix protein 2 (M2). Moreover, newly identified viruses (such as coronaviruses) can be difficult to treat because they are not sufficiently characterized. The emergence of drug resistance and newly identified viruses highlights the need for the development of novel antiviral strategies. Host cell targeting may reduce or avoid the emergence of escape mutants, but could create a “sink” due to widespread expression and raise the concern for toxicity. A number of respiratory virus fusion proteins have been shown to require cleavage by host protease(s) for activation (Shirato et al. Clinical Isolates of Human Coronavirus 229E Bypass the Endosome for Cell Entry. Journal of Virology. 91, e01387-16 (2017); Reinke et al., Different residues in the SARS-CoV spike protein determine cleavage and activation by the host cell protease TMPRSS2. PLoS ONE. 12, e0179177 (2017); Zhou et al., Protease inhibitors targeting coronavirus and filovirus entry. Antiviral Research. 116, 76-84 (2015); Zmora et al. TMPRSS2 Isoform 1 Activates Respiratory Viruses and Is Expressed in Viral Target Cells. PLoS ONE. 10, e0138380 (2015)), including influenza (Zmora et al., Non-human primate orthologues of TMPRSS2 cleave and activate the influenza virus hemagglutinin. PLoS ONE. 12, e0176597 (2017); Bottcher-Friebertshauser et al., Inhibition of influenza virus infection in human airway cell cultures by an antisense peptide-conjugated morpholino oligomer targeting the hemagglutinin-activating protease TMPRSS2. Journal of Virology. 85, 1554-1562 (2011); Bertram et al., TMPRSS2 and TMPRSS4 facilitate trypsin-independent spread of influenza virus in Caco-2 cells. Journal of Virology. 84, 10016-10025 (2010); Tarnow et al., TMPRSS2 is a host factor that is essential for pneumotropism and pathogenicity of H7N9 influenza A virus in mice. Journal of Virology (2014), May; 88(9):4744-51) and coronavirus (Heurich et al., TMPRSS2 and ADAM17 cleave ACE2 differentially and only proteolysis by TMPRSS2 augments entry driven by the severe acute respiratory syndrome coronavirus spike protein. Journal of Virology. 88, 2202-2013 (2014)).

Influenza A hemagglutinin precursor (HAG) requires cleavage by a host serine protease, to HA1 and HA2, for activation. For example, transmembrane protease, serine 2; TMPRSS2, TMPRSS4 and TMPRSS11D as well as human airway trypsin-like protease (HAT) have been implicated in HA cleavage (Bertram et al., TMPRSS2 and TMPRSS4 facilitate trypsin-independent spread of influenza virus in Caco-2 cells. Journal of Virology. 84, 10016-10025 (2010); Bottcher et al., Proteolytic Activation of Influenza Viruses by Serine Proteases TMPRSS2 and HAT from Human Airway Epithelium. Journal of Virology. 2006 Oct; 80(19):9896-8; International patent application publication no. WO2017/151453). TMPRSS2 also cleaves and activates the spike protein in coronaviruses such as the severe acute respiratory syndrome coronavirus (SARS-CoV) (Heurich et al., TMPRSS2 and ADAM17 cleave ACE2 differentially and only proteolysis by TMPRSS2 augments entry driven by the severe acute respiratory syndrome coronavirus spike protein. Journal of Virology. 88, 2202-2013 (2014)). Additionally, TMPRSS2 is a target for anti-cancer therapy. See e.g., WO2008127347 and WO2002004953. A fusion between TMPRSS2 and ERG (TMPRSS2:ERG) is a gene fusion known to be a major driver of prostate carcinogenesis which is triggered by the ERα and repressed by the ERβ. Bonkhoff, Estrogen receptor signaling in prostate cancer: Implications for carcinogenesis and tumor progression, Prostate 78(1): 2-10 (2018).

### SUMMARY OF THE INVENTION

Although there are small molecule inhibitors of TMPRSS2 and research antibodies, useful, for example, for immunohistochemistry, there is a need in the art for neutralizing therapeutic anti-TMPRSS2 antibodies and their use for treating or preventing viral infection. See e.g., Shen et al. Biochimie 142: 1-10 (2017), WO2008127347; WO2002004953; U.S. Pat. No. 9,498,529; antibody ab92323, available from Abcam (Cambridge, MA) or antibodies sc-515727 and sc-101847 available from Santa Cruz Biotech (Dallas, TX). The present invention addresses this need, in part, by providing human anti-human TMPRSS2 antibodies, such as mAb8021, mAb8029, and mAb8028, and combinations thereof including, for example, combinations with other therapeutics such as anti-influenza HA antibodies (e.g., Group I HA or Group II HA) or anti-coronavirus spike protein antibodies (e.g., Severe Acute Respiratory Syndrome (SARS) coronavirus (SARS-CoV), Middle East Respiratory Syndrome (MERS) coronavirus (MERS-CoV), and SARS-CoV-2 (the coronavirus responsible for COVID-19, previously referred to as 2019-nCoV)), and methods of use thereof for treating viral infections.

The present invention provides neutralizing human antigen-binding proteins that specifically bind to human TMPRSS2, for example, antibodies or antigen-binding fragments thereof. In some aspects, the antibody or antigen-binding fragment thereof is an isolated recombinant antibody or antigen-binding fragment thereof. In some embodiments, an antigen-binding protein comprises: (a) the CDR-H1, CDR-H2, and CDR-H3 of an immunoglobulin heavy chain that comprises the amino acid sequence set forth in SEQ ID NO: 2, 22, or 42; and/or (b) the CDR-L1, CDR-L2, and CDR-L3 of an immunoglobulin light chain that comprises the amino acid sequence set forth in SEQ ID NO: 10, 30, or 50. In an embodiment of the invention, an antigen-binding protein comprises: (a) a light chain immunoglobulin variable region comprising an amino acid sequence having at least 90% amino acid sequence identity to the amino acid sequence set forth in SEQ ID NO: 10, 30, or 50; and/or (b) a heavy chain immunoglobulin variable region comprising an amino acid sequence having at least 90% amino acid sequence identity to the amino acid sequence set forth in SEQ ID NO: 2, 22, or 42. In an embodiment of the invention, the present invention provides antigen-binding protein comprising: (a) CDR-L1, CDR-L2 and CDR-L3 of a light chain immunoglobulin comprising an amino acid sequence set forth in SEQ ID NO: 10, 30, or 50 and at least 90% amino acid sequence identity to the amino acid sequence set forth in SEQ ID NO: 10, 30, or 50; and/or (b) CDR-H1, CDR-H2 and CDR-H3 of a heavy chain immunoglobulin comprising an amino acid sequence set forth in SEQ ID NO: 2, 22, or 42 and at least 90% amino acid sequence identity to the amino acid sequence set forth in SEQ ID NO: 2, 22, or 42. For example, in an embodiment of the invention, the antigen-binding protein comprises a heavy chain immunoglobulin variable region that comprises (a) a CDR-H1 comprising the amino acid sequence: F T F R S Y D (SEQ ID NO: 4); (b) a CDR-H2 comprising the amino acid sequence: G S A G D T (SEQ ID NO: 6); (c) a CDR-H3 comprising the amino acid sequence: R V G D W G S G Y L D Y (SEQ ID NO: 8); and a light chain immunoglobulin variable region that comprises (a) a CDR-L1 comprising the amino acid sequence: S I S I Y (SEQ ID NO: 12); (b) a CDR-L2 comprising the amino acid sequence: A S (SEQ ID NO: 14); and/or (c) a CDR-L3 comprising the amino acid sequence: Q S Y G T P F T (SEQ ID NO: 16). The present invention also provides an antigen-binding protein comprising: (a) a heavy chain immunoglobulin that comprises the amino acid sequence set forth in SEQ ID NO: 18, 38, or 58; and/or (b) a light chain immunoglobulin that comprises the amino acid sequence set forth in SEQ ID NO: 20, 40, or 60.

In some aspects, the antigen-binding protein, e.g., the isolated recombinant antibody or antigen-binding fragment thereof, that specifically binds to human transmembrane protease, serine 2 (TMPRSS2), has one or more of the following characteristics:
(a) binds to TMPRSS2 with an EC₅₀ of less than about 10⁻⁹ M;
(b) demonstrates an increase in survival in a coronavirus-infected animal after administration to said coronavirus-infected animal, as compared to a comparable coronavirus-infected animal without said administration; and/or
(c) comprises
   (i) three heavy chain complementarity determining regions (CDRs) (CDR-H1, CDR-H2, and CDR-H3) contained within a heavy chain variable region (HCVR) comprising an amino acid sequence having at least about 90% sequence identity to the HCVR set forth in SEQ ID NO: 2; and three light chain CDRs (CDR-L1, CDR-L2, and CDR-L3) contained within a light chain variable region (LCVR) comprising an amino acid sequence having at least about 90% sequence identity to the LCVR set forth in SEQ ID NO: 10; or
   (ii) three heavy chain CDRs (CDR-H1, CDR-H2, and CDR-H3) contained within an HCVR comprising an amino acid sequence having at least about 90% sequence identity to the HCVR set forth in SEQ ID NO: 22; and three light chain CDRs (CDR-L1, CDR-L2, and CDR-L3) contained within an LCVR comprising an amino acid sequence having at least about 90% sequence identity to the LCVR set forth in SEQ ID NO: 30; or
   (iii) three heavy chain CDRs (CDR-H1, CDR-H2, and CDR-H3) contained within an HCVR comprising an amino acid sequence having at least about 90% sequence identity to the HCVR set forth in SEQ ID NO: 42; and three light chain CDRs (CDR-L1, CDR-L2, and CDR-L3) contained within an LCVR comprising an amino acid sequence having at least about 90% sequence identity to the LCVR set forth in SEQ ID NO: 50.

In some aspects, the antigen-binding protein, e.g., the antibody or antigen-binding fragment thereof, comprises the three heavy chain CDRs (CDR-H1, CDR-H2, and CDR-H3) contained within the HCVR comprising an amino acid sequence having at least about 90% sequence identity to the HCVR set forth in SEQ ID NO: 2; and the three light chain CDRs (CDR-L1, CDR-L2, and CDR-L3) contained within the LCVR comprising an amino acid sequence having at least about 90% sequence identity to the LCVR set forth in SEQ ID NO: 10.

In some aspects, the antigen-binding protein, e.g., the antibody or antigen-binding fragment thereof, comprises: (a) an immunoglobulin heavy chain variable region comprising the CDR-H1, CDR-H2, and CDR-H3 of an immunoglobulin heavy chain that comprises the amino acid sequence set forth in SEQ ID NO: 2; and/or (b) an immunoglobulin light chain variable region comprising the CDR-L1, CDR-L2, and CDR-L3 of an immunoglobulin light chain that comprises the amino acid sequence set forth in SEQ ID NO: 10.

In some aspects, the antigen-binding protein, e.g., the antibody or antigen-binding fragment thereof, comprises: (a) a heavy chain immunoglobulin variable region comprising an amino acid sequence having at least 90% amino acid sequence identity to the amino acid sequence set forth in SEQ ID NO: 2; and/or (b) a light chain immunoglobulin variable region comprising an amino acid sequence having at least 90% amino acid sequence identity to the amino acid sequence set forth in SEQ ID NO: 10.

In some aspects, the antigen-binding protein, e.g., the antibody or antigen-binding fragment thereof, comprises: a heavy chain immunoglobulin variable region that comprises
(a) a CDR-H1 comprising the amino acid sequence of SEQ ID NO: 4,
(b) a CDR-H2 comprising the amino acid sequence of SEQ ID NO: 6, and/or
(c) a CDR-H3 comprising the amino acid sequence of SEQ ID NO: 8; and/or
a light chain immunoglobulin variable region that comprises
(a) a CDR-L1 comprising the amino acid sequence of SEQ ID NO: 12,
(b) a CDR-L2 comprising the amino acid sequence of SEQ ID NO: 14, and/or
(c) a CDR-L3 comprising the amino acid sequence of SEQ ID NO: 16.

In some aspects, the antigen-binding protein, e.g., the antibody or antigen-binding fragment thereof, comprises: (a) a heavy chain immunoglobulin that comprises the amino acid sequence set forth in SEQ ID NO: 18, or an immunoglobulin heavy chain variable region that comprises the amino acid sequence set forth in SEQ ID NO: 2; and/or (b) a light chain immunoglobulin that comprises the amino acid sequence set forth in SEQ ID NO: 20, or an immunoglobulin light chain variable region that comprises the amino acid sequence set forth in SEQ ID NO: 10.

In some aspects, the antigen-binding protein, e.g., the antibody or antigen-binding fragment thereof, comprises the three heavy chain CDRs (CDR-H1, CDR-H2, and CDR-H3) contained within the HCVR comprising an amino acid sequence having at least about 90% sequence identity to the HCVR set forth in SEQ ID NO: 22; and the three light chain CDRs (CDR-L1, CDR-L2, and CDR-L3) contained within the LCVR comprising an amino acid sequence having at least about 90% sequence identity to the LCVR set forth in SEQ ID NO: 30.

In some aspects, the antigen-binding protein, e.g., the antibody or antigen-binding fragment thereof, comprises (a) an immunoglobulin heavy chain variable region comprising the CDR-H1, CDR-H2, and CDR-H3 of an immunoglobulin heavy chain that comprises the amino acid sequence set forth in SEQ ID NO: 22; and/or (b) an immunoglobulin light chain variable region comprising the CDR-L1, CDR-L2, and CDR-L3 of an immunoglobulin light chain that comprises the amino acid sequence set forth in SEQ ID NO: 30.

In some aspects, the antigen-binding protein, e.g., the antibody or antigen-binding fragment thereof, comprises (a) a heavy chain immunoglobulin variable region comprising an amino acid sequence having at least 90% amino acid sequence identity to the amino acid sequence set forth in SEQ ID NO: 22; and/or (b) a light chain immunoglobulin variable region comprising an amino acid sequence having at least 90% amino acid sequence identity to the amino acid sequence set forth in SEQ ID NO: 30.

In some aspects, the antigen-binding protein, e.g., the antibody or antigen-binding fragment thereof, comprises: a heavy chain immunoglobulin variable region that comprises
(a) a CDR-H1 comprising the amino acid sequence of SEQ ID NO: 24,
(b) a CDR-H2 comprising the amino acid sequence of SEQ ID NO: 26, and/or
(c) a CDR-H3 comprising the amino acid sequence of SEQ ID NO: 28; and/or
a light chain immunoglobulin variable region that comprises
(a) a CDR-L1 comprising the amino acid sequence of SEQ ID NO: 32,
(b) a CDR-L2 comprising the amino acid sequence of SEQ ID NO: 34, and/or
(c) a CDR-L3 comprising the amino acid sequence of SEQ ID NO: 36.

In some aspects, the antigen-binding protein, e.g., the antibody or antigen-binding fragment thereof, comprises: (a) a heavy chain immunoglobulin that comprises the amino acid sequence set forth in SEQ ID NO: 38, or an immunoglobulin heavy chain variable region that comprises the amino acid sequence set forth in SEQ ID NO: 22; and/or (b) a light chain immunoglobulin that comprises the amino acid sequence set forth in SEQ ID NO: 40, or an immunoglobulin light chain variable region that comprises the amino acid sequence set forth in SEQ ID NO: 30.

In some aspects, the antigen-binding protein, e.g., the antibody or antigen-binding fragment thereof, comprises: the three heavy chain CDRs (CDR-H1, CDR-H2, and CDR-H3) contained within the HCVR comprising an amino acid sequence having at least about 90% sequence identity to the HCVR set forth in SEQ ID NO: 42; and the three light chain CDRs (CDR-L1, CDR-L2, and CDR-L3) contained within the LCVR comprising an amino acid sequence having at least about 90% sequence identity to the LCVR set forth in SEQ ID NO: 50.

In some aspects, the antigen-binding protein, e.g., the antibody or antigen-binding fragment thereof, comprises: (a) an immunoglobulin heavy chain variable region comprising the CDR-H1, CDR-H2, and CDR-H3 of an immunoglobulin heavy chain that comprises the amino acid sequence set forth in SEQ ID NO: 42; and/or (b) an immunoglobulin light chain variable region comprising the CDR-L1, CDR-L2, and CDR-L3 of an immunoglobulin light chain that comprises the amino acid sequence set forth in SEQ ID NO: 50.

In some aspects, the antigen-binding protein, e.g., the antibody or antigen-binding fragment thereof, comprises: (a) a heavy chain immunoglobulin variable region comprising an amino acid sequence having at least 90% amino acid sequence identity to the amino acid sequence set forth in SEQ ID NO: 42; and/or (b) a light chain immunoglobulin variable region comprising an amino acid sequence having at least 90% amino acid sequence identity to the amino acid sequence set forth in SEQ ID NO: 30.

In some aspects, the antigen-binding protein, e.g., the antibody or antigen-binding fragment thereof, comprises:
(a) a CDR-H1 comprising the amino acid sequence of SEQ ID NO: 44,
(b) a CDR-H2 comprising the amino acid sequence of SEQ ID NO: 46, and/or
(c) a CDR-H3 comprising the amino acid sequence of SEQ ID NO: 48; and/or
a light chain immunoglobulin variable region that comprises
(a) a CDR-L1 comprising the amino acid sequence of SEQ ID NO: 52,
(b) a CDR-L2 comprising the amino acid sequence of SEQ ID NO: 54, and/or
(c) a CDR-L3 comprising the amino acid sequence of SEQ ID NO: 56.

In some aspects, the antigen-binding protein, e.g., the antibody or antigen-binding fragment thereof, comprises: (a) a heavy chain immunoglobulin that comprises the amino acid sequence set forth in SEQ ID NO: 58, or an immunoglobulin heavy chain variable region that comprises the amino acid sequence set forth in SEQ ID NO: 42; and/or (b) a light chain immunoglobulin that comprises the amino acid sequence set forth in SEQ ID NO: 60, or an immunoglobulin light chain variable region that comprises the amino acid sequence set forth in SEQ ID NO: 50.

The present invention also provides any anti-TMPRSS2 antigen-binding protein that competes with any antigen-binding protein that is set forth herein for binding to TMPRSS2 (e.g., as determined by use of using a real time, label-free bio-layer interferometry assay, e.g., on an Octet RED384 biosensor (Pall ForteBio Corp.)); or which binds to the same or an overlapping epitope on TMPRSS2 (or a fragment thereof) as any antigen-binding protein that is set forth herein.

The present invention also provides multispecific antigen-binding proteins that bind to TMPRSS2 and another antigen or to TMPRSS2 at a different epitope. For example, the multispecific molecule comprises (a) a first antigen-binding domain that binds specifically to TMPRSS2; and (b) a second antigen-binding domain that binds specifically to another antigen or to TMPRSS2 or to an epitope which differs from that of the first antigen-binding domain. In some aspects, the multispecific antigen-binding protein is an antibody or antigen-binding fragment thereof.

The present invention also provides any anti-TMPRSS2 antigen-binding protein (e.g., an antibody or antigen-binding fragment, e.g., comprising a sequence set forth herein) that comprises one or more of the following properties:
Inhibits growth of influenza virus (e.g., A/Puerto Rico/08/1934 (H1N1)) or coronavirus (e.g., SARS-CoV, MERS-CoV, or SARS-CoV-2) in TMPRSS2-expressing cells (e.g., Calu-3 cells);
Binds to the surface of TMPRSS-expressing cells;
Does not significantly bind to MDCK/Tet-on cells which do not express TMPRSS2;
Limits spread of influenza virus infection or coronavirus infection of cells in vitro; and/or
Protects a mouse engineered to express the human TMPRSS2 protein from death and/or weight loss caused by influenza virus infection or coronavirus infection.

The present invention also provides a complex comprising any antigen-binding protein set forth herein bound to a TMPRSS2 polypeptide, e.g., in vitro or in the body of a subject.

The present invention also provides a method for making an anti-TMPRSS2 antigen-binding protein set forth herein (e.g., mAb8021, mAb8028, or mAb8029) or immunoglobulin chain thereof comprising: (a) introducing one or more polynucleotides encoding a light and/or a heavy immunoglobulin chain of the said antigen-binding protein; (b) culturing the host cell (e.g., CHO cell, Pichia cell or Pichia pastoris cell) under conditions favorable to expression of the polynucleotide; and (c) optionally, isolating the antigen-binding protein or immunoglobulin chain from the host cell and/or medium in which the host cell is grown. An antigen-binding protein or immunoglobulin chain which is a product of such a method is part of the present invention.

A polypeptide comprising: (a) CDR-H1, CDR-H2, and CDR-H3 of a V_{H} domain of an antibody or antigen-binding fragment that comprises the amino acid sequence set forth in any one of SEQ ID NO: 2, 22, or 42; or (b) CDR-L1, CDR-L2, and CDR-L3 of a V_{L} domain of an immunoglobulin chain that comprises the amino acid sequence set forth in SEQ ID NO: 10, 30, or 50, also forms part of the present invention. In some embodiments, provided herein is a polynucleotide encoding said polypeptide.

A polypeptide (e.g., an immunoglobulin) comprising: (a) CDR1, CDR2, and CDR3 of a V_{H} domain of an immunoglobulin chain that comprises the amino acid sequence set forth in SEQ ID NO: 2; or (b) CDR1, CDR2, and CDR3 of a V_{L} domain of an immunoglobulin chain that comprises the amino acid sequence set forth in SEQ ID NO: 10 (e.g., wherein the polypeptide is in a host cell) also forms part of the present invention. Similarly, the present invention provides a polypeptide comprising (a) CDR1, CDR2, and CDR3 of a V_{H} domain of an immunoglobulin chain that comprises the amino acid sequence set forth in SEQ ID NO: 22; or (b) CDR1, CDR2, and CDR3 of a V_{L} domain of an immunoglobulin chain that comprises the amino acid sequence set forth in SEQ ID NO: 30 (e.g., wherein the polypeptide is in a host cell). Similarly, the present invention provides a polypeptide comprising (a) CDR1, CDR2, and CDR3 of a V_{H} domain of an immunoglobulin chain that comprises the amino acid sequence set forth in SEQ ID NO: 42; or (b) CDR1, CDR2, and CDR3 of a V_{L} domain of an immunoglobulin chain that comprises the amino acid sequence set forth in SEQ ID NO: 50 (e.g., wherein the polypeptide is in a host cell). In some embodiments, provided herein is a polynucleotide encoding said polypeptide.

The present invention also provides a polynucleotide (e.g., DNA or RNA) that encoded a polypeptide of the present invention. In an embodiment of the invention, the polynucleotide encodes two different immunoglobulin chains (e.g., heavy and light). In an embodiment of the invention, one polynucleotide encodes a light immunoglobulin chain and another polynucleotide encodes a heavy immunoglobulin chain, e.g., wherein the chains are in a host cell or are in a vessel. For example, the polynucleotide is in a vector (e.g., a plasmid) and/or is integrated into a host cell chromosome.

The present invention also provides a vector comprising a polynucleotide as described herein.

Host cells (e.g., CHO cell, Pichia cell or Pichia pastoris cell) of the present invention may include an anti-TMPRSS2 antigen-binding protein (e.g., mAb8021, mAb8028, or mAb8029), polypeptide thereof or polynucleotide encoding such a polypeptide and/or a vector including such a polynucleotide.

The present invention also provides a composition or kit comprising an anti-TMPRSS2 antigen-binding protein set forth herein (e.g., mAb8021, mAb8028, or mAb8029) in association with a further therapeutic agent (e.g., an anti-viral drug and/or a vaccine). For example, the composition may be a pharmaceutical composition comprising the antigen-binding protein and pharmaceutically acceptable carrier and, optionally, a further therapeutic agent. The further therapeutic agent may be remdesivir, chloroquine, lopinavir, ritonavir, ribavirin, ledipasvir, sofosbuvir, a combination of ledipasvir and sofosbuvir, oseltamivir, zanamivir, ribavirin and interferon-alpha2b, interferon-alpha2a and/or an antibody or antigen-binding fragment thereof that specifically binds to influenza HA or coronavirus spike protein. In an embodiment of the invention, the further therapeutic agent is an antibody or antigen binding fragment thereof selected from the group consisting of H1H14611N2; H1H14612N2; H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; and H1H18335B, as set forth in International patent application publication no. WO2016/100807. In an embodiment of the invention, the further therapeutic agent is an antibody or antigen binding fragment thereof selected from the group consisting of H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2, as set forth in International patent application publication no. WO/2015/179535.

In an embodiment of the invention, a further therapeutic agent which is provided in association with an anti-TMPRSS2 antigen-binding protein is an antibody or antigen-binding fragment that binds to influenza Group II HA protein, such as H1H14611N2; or an antibody or fragment that comprises V_{H} and V_{L} of H1H14611N2; or a heavy chain immunoglobulin comprising CDR-H1, CDR-H2 and CDR-H3 of H1H14611N2 and a light chain immunoglobulin comprising CDR-L1, CDR-L2 and CDR-L3 of H1H14611N2.

In an embodiment of the invention, a further therapeutic agent which is provided in association with an anti-TMPRSS2 antigen-binding protein is an antibody or antigen-binding fragment that binds to influenza Group II HA protein, such as H1H14612N2; or an antibody or fragment that comprises V_{H} and V_{L} of H1H14612N2; or a heavy chain immunoglobulin comprising CDR-H1, CDR-H2 and CDR-H3 of H1H14612N2 and a light chain immunoglobulin comprising CDR-L1, CDR-L2 and CDR-L3 of H1H14612N2.

In an embodiment of the invention, a further therapeutic agent which is provided in association with an anti-TMPRSS2 antigen-binding protein is an antibody or antigen-binding fragment that binds to influenza Group I HA protein, such as H1H11729P; or an antibody or fragment that comprises V_{H} and V_{L} of H1H11729P; or a heavy chain immunoglobulin comprising CDR-H1, CDR-H2 and CDR-H3 of H1H11729P and a light chain immunoglobulin comprising CDR-L1, CDR-L2 and CDR-L3 of H1H11729P.

The present invention also provides a vessel or injection device that comprises an anti-TMPRSS2 antigen-binding protein (e.g., mAb8021, mAb8028, or mAb8029) or composition thereof (e.g., pharmaceutical composition).

The present invention also provides a method for treating or preventing a viral infection other than an influenza virus infection, in a subject (e.g., a human) in need thereof, comprising administering a therapeutically effective amount of anti-TMPRSS2 antigen-binding protein set forth herein (e.g., mAb8021, mAb8028, or mAb8029).

The present invention also provides a method for treating or preventing cancer (e.g., prostate cancer, colon cancer, lung cancer, pancreas cancer, urinary tract cancer, breast cancer, ovarian cancer, prostate adenocarcinoma, renal cell carcinoma, colorectal adenocarcinoma, lung adenocarcinoma, lung squamous cell carcinoma, and/or pleural mesothelioma) or infection, e.g., a viral infection, e.g., an infection with an influenza virus, coronavirus, SARS-CoV, MERS-CoV, SARS-CoV-2, parainfluenza virus, human metapneumovirus or hepatitis C virus (HCV), in a subject (e.g., a human) in need thereof, comprising administering a therapeutically effective amount of anti-TMPRSS2 antigen-binding protein set forth herein (e.g., mAb8021, mAb8028, or mAb8029).

In some aspects, the method comprises administering the antigen-binding protein in association with one or more further therapeutic agents (e.g., anti-viral drug and/or a vaccine). In an embodiment of the invention, a further therapeutic agent is a member selected from the group consisting of: remdesivir, chloroquine, lopinavir, ritonavir, ribavirin, ledipasvir, sofosbuvir, a combination of ledipasvir and sofosbuvir, oseltamivir, zanamivir, ribavirin and interferon-alpha2b, interferon-alpha2a and an antibody or antigen-binding fragment thereof that specifically binds to influenza HA. In an embodiment of the invention, a further therapeutic agent is an antibody or antigen binding fragment thereof selected from the group consisting of H1H14611N2; H1H14612N2; H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; and H1H18335B, as set forth in International patent application publication no. WO2016/100807. In an embodiment of the invention, the further therapeutic agent is an antibody or antigen binding fragment thereof selected from the group consisting of H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2, as set forth in International patent application publication no. WO/2015/179535.

The present invention also provides a method for administering an anti-TMRPSS2 antigen-binding protein (e.g., mAb8021, mAb8028, or mAb8029) set forth herein into the body of a subject (e.g., a human) comprising injecting the antigen-binding protein into the body of the subject. In some aspects, the antigen-binding protein is injected into the body of the subject parenterally (e.g., subcutaneously, intravenously or intramuscularly).

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

The term “coronavirus” or “CoV” refers to any virus of the coronavirus family, including but not limited to MERS-CoV, SARS-CoV, and SARS-CoV-2. SARS-CoV-2 refers to the newly-emerged coronavirus that causes COVID-19, This virus was initially identified as the cause of a serious outbreak starting in Wuhan, China, and rapidly spread around the globe. It binds via the viral spike protein to human host cell receptor angiotensin-converting enzyme 2 (ACE2). The spike protein also binds to and is cleaved by TMPRSS2, which activates the spike protein for membrane fusion of the virus.

The term “CoV-S”, also called “S protein” refers to the spike protein of a coronavirus, and can refer to specific S proteins such as MERS-CoV-S, SARS-CoV-S, and SARS-CoV-2-S. The SARS-CoV-2 spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 subunit. The amino acid sequence of full-length SARS-CoV-2 spike protein is exemplified by the amino acid sequence provided in SEQ ID NO: 61. The term “CoV-S” includes protein variants of CoV spike protein isolated from different CoV isolates as well as recombinant CoV spike protein or a fragment thereof. The term also encompasses CoV spike protein or a fragment thereof coupled to, for example, a histidine tag, mouse or human Fc, or a signal sequence such as ROR1.

The term “coronavirus infection” or “CoV infection,” as used herein, refers to infection with a coronavirus such as MERS-CoV, SARS-CoV, or SARS-CoV-2. The term includes respiratory tract infection, often in the lower respiratory tract. The symptoms include high fever, cough, shortness of breath pneumonia, gastro-intestinal symptoms such as diarrhea, organ failure (kidney failure and renal dysfunction), septic shock and death in severe cases. Infection with SARS-CoV-2 can cause Coronavirus Disease 19 (COVID-19), which may cause symptoms such as fever, chills, shortness of breath, congestion, cough, fatigue, body/muscle aches, and loss of taste and/or smell.

The term “influenza hemagglutinin”, also called “influenza HA” is a trimeric glycoprotein found on the surface of influenza virions, which mediates viral attachment (via HA1 binding to α-2,3- and α-2,6-sialic acids) and entry (through conformational change) into host cells. The HA is comprised of two structural domains: a globular head domain containing the receptor binding site (subject to high frequency of antigenic mutations) and the stem region (more conserved among various strains of influenza virus). The influenza HA is synthesized as a precursor (HAG) that undergoes proteolytic processing to produce two subunits (HA1 and HA2) which associate with one another to form the stem/globular head structure. The viral HA is the most variable antigen on the virus and the stem (HA2) is highly conserved within each group.

The term “influenza neuraminidase”, also called “influenza NA” is an exosialidase (EC 3.2.1.18) which cleaves α-ketosidic linkage between the sialic (N-acetylneuraminic) acid and an adjacent sugar residue.

The amino acid sequence of full-length Influenza HA is exemplified by the amino acid sequence of influenza isolate H1N1 A/California/04/2009 provided in GenBank as accession number FJ966082.1. The term “influenza-HA” also includes protein variants of influenza HA isolated from different influenza isolates, e.g., GQ149237.1, NC_002017, KM972981.1, etc. The term “influenza-HA” also includes recombinant influenza HA or a fragment thereof. The term also encompasses influenza HA or a fragment thereof coupled to, for example, histidine tag, mouse or human Fc, or a signal sequence.

An anti-TMPRSS2 “antigen-binding protein” is a polypeptide or complex of more than one polypeptide (e.g., a tetrameric IgG antibody) that binds specifically to TMPRSS2 polypeptide, for example, an anti-TMPRSS2 antibody or antigen-binding fragment whether monospecific or multispecific.

### TMPRSS2

TMPRSS2 (Transmembrane protease serine 2) is a protein, located on human chromosome 21, that belongs to the serine protease family (type II transmembrane serine proteases (TTSPs)) which is important for influenza virus infectivity. TMPRSS2 has been demonstrated to mediate cleavage of influenza virus HAG to HA1 and HA2.

The human TMPRSS2 gene encodes a predicted protein of 492 amino acids which anchors to the plasma membrane. The protein converts to its mature form through autocatalytic cleavage between Arg255 and Ile256. After cleavage, the mature proteases are mostly membrane bound, yet a portion of them may be liberated into the extracellular milieu.

In an embodiment of the invention, human TMPRSS2 (V160M) comprises the amino acid sequence: MALNSGSPPAIGPYYENHGYQPENPYPAQPTVVPTVYEVHPAQYYPSPVPQYAPRVLTQ ASNPVVCTQPKSPSGTVCTSKTKKALCITLTLGTFLVGAALAAGLLWKFMGSKCSNSGI ECDSSGTCINPSNWCDGVSHCPGGEDENRCVRLYGPNFILQMYSSQRKSWHPVCQDDW NENYGRAACRDMGYKNNFYSSQGIVDDSGSTSFMKLNTSAGNVDIYKKLYHSDACSSK AVVSLRCIACGVNLNSSRQSRIVGGESALPGAWPWQVSLHVQNVHVCGGSIITPEWIVT AAHCVEKPLNNPWHWTAFAGILRQSFMFYGAGYQVEKVISHPNYDSKTKNNDIALMK LQKPLTFNDLVKPVCLPNPGMMLQPEQLCWISGWGATEEKGKTSEVLNAAKVLLIETQ RCNSRYVYDNLITPAMICAGFLQGNVDSCQGDSGGPLVTSKNNIWWLIGDTSWGSGCA KAYRPGVYGNVMVFTDWIYRQMRADG (SEQ ID NO: 63; methionine 160 in bold font). In an embodiment of the invention, the TMPRSS2 polypeptide does not comprise the V160M mutation. See also NCBI Accession No. NM_005656.3.

In an embodiment of the invention, Macaca mulatta TMPRSS2 (S129L, N251S, I415V, R431Q, D492G) comprises the amino acid sequence: MALNSGSPPGVGPYYENHGYQPENPYPAQPTVAPNVYEVHPAQYYPSPVPQYTPRVLT HASNPAVCRQPKSPSGTVCTSKTKKALCVTMTLGAVLVGAALAAGLLWKFMGSKCSD SGIECDSSGTCISLSNWCDGVSHCPNGEDENRCVRLYGPNFILQVYSSQRKSWHPVCRD DWNENYARAACRDMGYKNSFYSSQGIVDNSGATSFMKLNTSAGNVDIYKKLYHSDAC SSKAVVSLRCIACGVRSNLSRQSRIVGGQNALLGAWPWQVSLHVQNIHVCGGSIITPEWI VTAAHCVEKPLNSPWQWTAFVGTLRQSSMFYEKGHRVEKVISHPNYDSKTKNNDIALM KLHTPLTFNEVVKPVCLPNPGMMLEPEQHCWISGWGATQEKGKTSDVLNAAMVPLIEP RRCNNKYVYDGLITPAMICAGFLQGTVDSCQGDSGGPLVTLKNDVWWLIGDTSWGSG CAQANRPGVYGNVTVFTDWIYRQMRADG (SEQ ID NO: 64). In an embodiment of the invention, the TMPRSS2 polypeptide does not comprise the S129L, N251S, I415V, R431Q and/or D492G mutation.

In an embodiment of the invention, Mus musculus TMPRSS2 mRNA comprises the nucleotide sequence set forth in NCBI Accession No. NM_015775.2.

### Viruses

The present invention includes methods for treating or preventing a viral infection in a subject. The term “virus” includes any virus whose infection in the body of a subject is treatable or preventable by administration of an anti-TMPRSS2 antibody or antigen-binding fragment thereof (e.g., wherein infectivity of the virus is at least partially dependent on TMPRSS2). In an embodiment of the invention, a “virus” is any virus that expresses HA0, spike protein (e.g., CoV-S), or another substrate of TMPRSS2 whose proteolytic cleavage is required for full infectivity of the virus against a cell in a host. The term “virus” also includes a TMPRSS2-dependent respiratory virus which is a virus that infects the respiratory tissue of a subject (e.g., upper and/or lower respiratory tract, trachea, bronchi, lungs) and is treatable or preventable by administration of an anti-TMPRSS2. For example, in an embodiment of the invention, virus includes influenza virus, coronavirus, SARS-CoV (severe acute respiratory syndrome coronavirus), MERS-CoV (Middle East Respiratory Syndrome (MERS) coronavirus), SARS-CoV-2, parainfluenza virus, Sendai virus (SeV), human metapneumovirus and/or hepatitis C virus (HCV). “Viral infection” refers to the invasion and multiplication of a virus in the body of a subject. The present invention includes embodiments with a proviso that “virus” excludes influenza virus, e.g., wherein viral infection excludes influenza virus infection.

Coronavirus virions are spherical with diameters of approximately 125 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. MERS-CoV (Middle East Respiratory Syndrome coronavirus), SARS-CoV (severe acute respiratory syndrome coronavirus), and SARS-CoV-2 belong to the coronavirus family. The initial attachment of the virion to the host cell is initiated by interactions between the S protein and its receptor. The sites of receptor binding domains (RBD) within the S1 region of a coronavirus S protein vary depending on the virus, with some having the RBD at the C-terminus of S1. The S-protein/receptor interaction is the primary determinant for a coronavirus to infect a host species and also governs the tissue tropism of the virus. Many coronaviruses utilize peptidases as their cellular receptor. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes.

Influenza viruses are members of the family Orthomyxoviridae. This family represents enveloped viruses the genome of which has segmented negative-sense single-strand RNA segments. There are four genera of this family: types A, B, C and Thogotovirus. The Influenza viruses classes, A, B and C, are based on core protein and are further divided into subtypes determined by the viral envelope glycoproteins hemagglutinin (HA) and neuraminidase (NA) (e.g., subtype A/H1N1). There are at least 18 influenza hemagglutinin (“HA”) protein subtypes (H1-H18 or HA1-HA18) and at least 11 influenza neuraminidase (NA) protein subtypes (N1-N11 or NA1-NA11) used to define influenza subtypes. Group 1 influenza has H1, H2, H5, H6, H8, H9, H11, H12, H13, H16, H17 and H18 subtypes and NA8, NA5, Na4 and NA1 subtypes. Group 2 has H3, H4, H7, H10, H14 and H15 subtypes and NA6, NA9, NA7, NA2 and NA3 subtypes. Influenza A viruses infect a range of mammalian and avian species, whereas type B and C infections are largely restricted to humans. The eight genome segments of influenza A and B viruses are loosely encapsidated by the nucleoprotein.

There are now two genera of human parainfluenza virus (HPIV), respirovirus (HPIV-1 and HPIV-3) and rubulavirus (HPIV-2 and HPIV-4). Both genera (paramyxoviruses) can be separated morphologically from influenza virus.

Sendai virus, also known as murine parainfluenza virus, is the type species in the genus respirovirus, which also contains the species human parainfluenza virus 3, bovine parainfluenza virus 3, and human parainfluenza virus 1. TMPRSS2 Is an Activating Protease for Respiratory Parainfluenza Viruses such as parainfluenza viruses and Sendai virus (SeV). See et al. Abe et al., J. Virol. 87(21): 11930-11935 (2013).

Human metapneumovirus (HMPV) is classified as the first human member of the Metapneumovirus genus in the Pneumovirinae subfamily within the Paramyxoviridae family. It is an enveloped negative-sense single-stranded RNA virus. The RNA genome includes 8 genes coding for 9 different proteins. HMPV is identical in gene order to the avian pneumovirus (AMPV), which also belongs to the Metapneumovirus genus. TMPRSS2 is expressed in the human lung epithelium, cleaves the HMPV F protein efficiently and supports HMPV multiplication and may be involved in the development of lower respiratory tract illness in HMPV-infected patients. See et al. Shirogane et al. J Virol. 82(17): 8942-8946 (2008).

Hepatitis C virus (HCV) is a small, enveloped, positive-sense single-stranded RNA virus of the family Flaviviridae. HCV, with at least 6 genotypes and numerous subtypes, is a member of the hepacivirus genus. TMPRSS2 may activate HCV infection at the post-binding and entry stage. Esumi et al., Hepatology 61(2): 437-446 (2015).

### Anti-TMPRSS2 Antibodies and Antigen-Binding Fragments

The present invention provides antigen-binding proteins, such as antibodies and antigen-binding fragments thereof, that specifically bind to TMPRSS2 protein or an antigenic fragment thereof.

The term “antibody”, as used herein, refers to immunoglobulin molecules comprising four polypeptide chains, two heavy chains (HCs) and two light chains (LCs) inter-connected by disulfide bonds (i.e., “full antibody molecules”), as well as multimers thereof (e.g. IgM). Exemplary antibodies include, for example, mAb8021, mAb8028, and mAb8029. Each heavy chain comprises a heavy chain variable region (“HCVR” or “V_{H}”) (e.g., SEQ ID NO 2) and a heavy chain constant region (comprised of domains C_{H}1, C_{H}2 and C_{H}3). Each light chain is comprised of a light chain variable region (“LCVR or “V_{L}”) (e.g., SEQ ID NO 4) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) are identical to the human germline sequences, or are naturally or artificially modified.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light and heavy chains variable domains comprise FR1, CDR1, FR2, CDR2, FR3, CDR3 and FR4. In an embodiment of the invention, the assignment of amino acids to each domain is in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The present invention includes monoclonal anti-TMPRSS2 antigen-binding proteins, e.g., antibodies and antigen-binding fragments thereof, as well as monoclonal compositions comprising a plurality of isolated monoclonal antigen-binding proteins. The term “monoclonal antibody”, as used herein, refers to a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment of the invention, an anti-TMPRSS2 antigen-binding protein, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an antigen-binding protein, e.g., antibody or antigen-binding fragment comprises a light chain constant domain, e.g., of the type kappa or lambda.

The term “human” antigen-binding protein, such as an antibody, as used herein, includes antibodies having variable and constant regions derived from human germline immunoglobulin sequences whether in a human cell or grafted into a non-human cell, e.g., a mouse cell. See e.g., U.S. Pat. No. 8,502,018, 6,596,541 or 5,789,215. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include antibodies isolated from or generated in a human subject. See below.

The present invention includes anti-TMPRSS2 chimeric antigen-binding proteins, e.g., antibodies and antigen-binding fragments thereof, and methods of use thereof. As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81: 6851-6855).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) expression system or isolated from a recombinant combinatorial human antibody library.

Recombinant anti-TMPRSS2 antigen-binding proteins, e.g., antibodies and antigen-binding fragments, disclosed herein may also be produced in an E. coli/T7 expression system. In this embodiment, nucleic acids encoding the anti-TMPRSS2 antibody immunoglobulin molecules of the invention (e.g., mAb8021, mAb8028, or mAb8029) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Thus, the present invention includes recombinant methods for making an anti-TMPRSS2 antigen-binding protein, such as an antibody or antigen-binding fragment thereof of the present invention, or an immunoglobulin chain thereof, comprising (i) introducing one or more polynucleotides (e.g., including the nucleotide sequence in any one or more of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, or 59) encoding light and/or heavy immunoglobulin chains, or CDRs, of the antigen-binding protein, e.g., mAb8021, mAb8028, or mAb8029, for example, wherein the polynucleotide is in a vector; and/or integrated into a host cell chromosome and/or is operably linked to a promoter; (ii) culturing the host cell (e.g., CHO or Pichia or Pichia pastoris) under condition favorable to expression of the polynucleotide and, (iii) optionally, isolating the antigen-binding protein, (e.g., antibody or fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods include those wherein only a heavy immunoglobulin chain or only a light immunoglobulin chain (e.g., any of those discussed herein including mature fragments and/or variable domains thereof) is expressed. Such chains are useful, for example, as intermediates in the expression of an antibody or antigen-binding fragment that includes such a chain. For example, the present invention also includes anti-TMPRSS2 antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 1 and a light chain immunoglobulin (or variable domain thereof or comprising the CDRs thereof) encoded by the nucleotide sequence set forth in SEQ ID NO: 9 which are the product of such production methods, and, optionally, the purification methods set forth herein. The present invention also includes anti-TMPRSS2 antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 21 and a light chain immunoglobulin (or variable domain thereof or comprising the CDRs thereof) encoded by the nucleotide sequence set forth in SEQ ID NO: 29 which are the product of such production methods, and, optionally, the purification methods set forth herein. The present invention also includes anti-TMPRSS2 antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 41 and a light chain immunoglobulin (or variable domain thereof or comprising the CDRs thereof) encoded by the nucleotide sequence set forth in SEQ ID NO: 49 which are the product of such production methods, and, optionally, the purification methods set forth herein. For example, in an embodiment of the invention, the product of the method is an anti-TMPRSS2 antigen-binding protein which is an antibody or fragment comprising a V_{H} comprising the amino acid sequence set forth in SEQ ID NO: 2 and a V_{L} comprising the amino acid sequence set forth in SEQ ID NO: 10; or comprising a HC comprising the amino acid sequence set forth in SEQ ID NO: 18 and a LC comprising the amino acid sequence set forth in SEQ ID NO: 20. In another embodiment of the invention, the product of the method is an anti-TMPRSS2 antigen-binding protein which is an antibody or fragment comprising a V_{H} comprising the amino acid sequence set forth in SEQ ID NO: 22 and a V_{L} comprising the amino acid sequence set forth in SEQ ID NO: 30; or comprising a HC comprising the amino acid sequence set forth in SEQ ID NO: 38 and a LC comprising the amino acid sequence set forth in SEQ ID NO: 40. In another embodiment of the invention, the product of the method is an anti-TMPRSS2 antigen-binding protein which is an antibody or fragment comprising a V_{H} comprising the amino acid sequence set forth in SEQ ID NO: 42 and a V_{L} comprising the amino acid sequence set forth in SEQ ID NO: 50; or comprising a HC comprising the amino acid sequence set forth in SEQ ID NO: 58 and a LC comprising the amino acid sequence set forth in SEQ ID NO: 60.

Eukaryotic and prokaryotic host cells, including mammalian cells, may be used as hosts for expression of an anti-TMPRSS2 antigen-binding protein. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a CHO cell) comprising an antigen-binding protein, such as mAb8021, mAb8028, or mAb8029; or a polynucleotide encoding such a polypeptide thereof.

The term “specifically binds” refers to those antigen-binding proteins (e.g., mAbs) having a binding affinity to an antigen, such as TMPRSS2 protein (e.g., human TMPRSS2), expressed as K_{D}, of at least about 10⁻⁸ M (e.g., 2.81×10⁻⁹ M; 9.31×10⁻⁹M; 10⁻⁹ M; 10⁻⁹ mM, 10⁻¹¹ M, or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present invention includes antigen-binding proteins that specifically bind to TMPRSS2 protein.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., as defined in WO08/020079 or WO09/138519) (e.g., monovalent nanobodies, bivalent nanobodies, etc.), small modular immunopharmaceuticals (SMIPs), and shark variable IgNAR domains, are also encompassed within the expression “antigen-binding fragment,” as used herein. In an embodiment of the invention, the antigen-binding fragment comprises three or more CDRs of mAb8021, mAb8028, or mAb8029 (e.g., CDR-H1, CDR-H2 and CDR-H3; or CDR-L1, CDR-L2 and CDR-L3).

An antigen-binding fragment of an antibody will, in an embodiment of the invention, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}I; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Antigen-binding proteins (e.g., antibodies and antigen-binding fragments) may be mono-specific or multi-specific (e.g., bi-specific). Multispecific antigen-binding proteins are discussed further herein.

In specific embodiments, antibody or antibody fragments of the invention may be conjugated to a moiety such a ligand or a therapeutic moiety (“immunoconjugate”), such as an anti-viral drug, a second anti-influenza antibody, or any other therapeutic moiety useful for treating a viral infection, e.g., influenza viral infection. See below.

The present invention also provides a complex comprising an anti-TMPRSS2 antigen-binding protein, e.g., antibody or antigen-binding fragment, discussed herein complexed with TMPRSS2 polypeptide or an antigenic fragment thereof and/or with a secondary antibody or antigen-binding fragment thereof (e.g., detectably labeled secondary antibody) that binds specifically to the anti-TMPRSS2 antibody or fragment. In an embodiment of the invention, the antibody or fragment is in vitro (e.g., is immobilized to a solid substrate) or is in the body of a subject. In an embodiment of the invention, the TMPRSS2 is in vitro (e.g., is immobilized to a solid substrate) or is on the surface of a cell or is in the body of a subject. Immobilized anti-TMRPSS2 antibodies and antigen-binding fragments thereof which are covalently linked to an insoluble matrix material (e.g., glass or polysaccharide such as agarose or sepharose, e.g., a bead or other particle thereof) are also part of the present invention; optionally, wherein the immobilized antibody is complexed with TMPRSS2 or antigenic fragment thereof or a secondary antibody or fragment thereof.

“Isolated” antigen-binding proteins, antibodies or antigen-binding fragments thereof, polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

The term “epitope” refers to an antigenic determinant (e.g., on TMPRSS2 polypeptide) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) (e.g., coversin) interacts is hydrogen/deuterium exchange detected by mass spectrometry. In general terms, the hydrogen/deuterium exchange method involves deuterium-labeling the protein of interest, followed by binding the antigen-binding protein, e.g., antibody or fragment or polypeptide, to the deuterium-labeled protein. Next, the TMPRSS2 protein/antigen-binding protein complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antigen-binding protein interface may retain deuterium and therefore exhibit relatively higher mass compared to amino acids not included in the interface. After dissociation of the antigen-binding protein (e.g., antibody or fragment or polypeptide), the target protein is subjected to protease cleavage and mass spectrometry analysis, thereby revealing the deuterium-labeled residues which correspond to the specific amino acids with which the antigen-binding protein interacts. See, e.g., Ehring (1999) Analytical Biochemistry 267: 252-259; Engen and Smith (2001) Anal. Chem. 73: 256A-265A.

The term “competes” as used herein, refers to an antigen-binding protein (e.g., antibody or antigen-binding fragment thereof) that binds to an antigen (e.g., TMPRSS2) and inhibits or blocks the binding of another antigen-binding protein (e.g., antibody or antigen-binding fragment thereof) to the antigen. The term also includes competition between two antigen-binding proteins e.g., antibodies, in both orientations, i.e., a first antibody that binds and blocks binding of second antibody and vice versa. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. In an embodiment of the invention, competition between a first and second anti-TMPRSS2 antigen-binding protein (e.g., antibody) is determined by measuring the ability of an immobilized first anti-TMPRSS2 antigen-binding protein (e.g., antibody) (not initially complexed with TMPRSS2 protein) to bind to soluble TMPRSS2 protein complexed with a second anti-TMPRSS2 antigen-binding protein (e.g., antibody). A reduction in the ability of the first anti-TMPRSS2 antigen-binding protein (e.g., antibody) to bind to the complexed TMPRSS2 protein, relative to uncomplexed TMPRSS2 protein, indicates that the first and second anti-TMPRSS2 antigen-binding proteins (e.g., antibodies) compete. The degree of competition can be expressed as a percentage of the reduction in binding. Such competition can be measured using a real time, label-free bio-layer interferometry assay, e.g., on an Octet RED384 biosensor (Pall ForteBio Corp.), ELISA (enzyme-linked immunosorbent assays) or SPR (surface plasmon resonance).

Binding competition between anti-TMPRSS2 antigen-binding proteins (e.g., monoclonal antibodies (mAbs)) can be determined using a real time, label-free bio-layer interferometry assay on an Octet RED384 biosensor (Pall ForteBio Corp.). For example, to determine competition between two anti-human TMPRSS2 monoclonal antibodies, the anti-TMPRSS2 mAb can be first captured onto anti-hFc antibody coated Octet biosensor tips (Pall ForteBio Corp., #18-5060) by submerging the tips into a solution of anti-human TMPRSS2 mAb (subsequently referred to as “mAb1”). As a positive-control for blocking, the antibody captured biosensor tips can then be saturated with a known blocking isotype control mAb (subsequently referred to as “blocking mAb”) by dipping into a solution of blocking mAb. To determine if mAb2 competes with mAb1, the biosensor tips can then be subsequently dipped into a co-complexed solution of human TMPRSS2 polypeptide and a second anti-human TMPRSS2 mAb (subsequently referred to as “mAb2”), that had been pre-incubated for a period of time and binding of mAb1 to the TMPRSS2 polypeptide can be determined. The biosensor tips can be washed in buffer in between every step of the experiment. The real-time binding response can be monitored during the course of the experiment and the binding response at the end of every step can be recorded.

For example, in an embodiment of the invention, the competition assay is conducted at 25° C. and pH about 7, e.g., 7.4, e.g., in the presence of buffer, salt, surfactant and a non-specific protein (e.g., bovine serum albumin).

Typically, an antibody or antigen-binding fragment of the invention which is modified in some way retains the ability to specifically bind to TMPRSS2, e.g., retains at least 10% of its TMPRSS2 binding activity (when compared to the parental antibody) when that activity is expressed on a molar basis. Preferably, an antibody or antigen-binding fragment of the invention retains at least 20%, 50%, 70%, 80%, 90%, 95% or 100% or more of the TMPRSS2 binding affinity as the parental antibody. It is also intended that an antibody or antigen-binding fragment of the invention can include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., mAb8021 V_{H}, V_{L}, HC, or LC, mAb8028 V_{H}, V_{L}, HC, or LC, or mAb8029 V_{H}, V_{L}, HC, or LC), refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., SEQ ID NO: 2, 10, 18, 20, 22, 30, 38, 40, 42, 50, 58, or 60); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

A “variant” of a polynucleotide refers to a polynucleotide comprising a nucleotide sequence that is at least about 70-99.9% (e.g., at least about 70, 72, 74, 75, 76, 79, 80, 81, 82, 83, 84, 85, 86, 87, 88, 89, 90, 91, 92, 93, 94, 95, 96, 97, 98, 99, 99.5, or 99.9%) identical to a referenced nucleotide sequence that is set forth herein (e.g., SEQ ID NO: 1, 9, 17, 19, 21, 29, 37, 39, 41, 49, 57, or 59); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear).

Anti-TMPRSS2 antigen-binding proteins, e.g., antibodies and antigen-binding fragments thereof of the present invention, in an embodiment of the invention, include a heavy chain immunoglobulin variable region having at least 70% (e.g., 80%, 85%, 90%, 95%, 99%) amino acid sequence identity to the amino acids set forth in SEQ ID NO: 2, 18, 22, 38, 42, or 58; and/or a light chain immunoglobulin variable region having at least 70% (e.g., 80%, 85%, 90%, 95%, 99%) amino acid sequence identity to the amino acids set forth in SEQ ID NO: 10, 20, 30, 40, 50, or 60.

In addition, a variant anti-TMPRSS2 antigen-binding protein may include a polypeptide comprising an amino acid sequence that is set forth herein except for one or more (e.g., 1, 2, 3, 4, 5, 6, 7, 8, 9 or 10) mutations such as, for example, missense mutations (e.g., conservative substitutions), non-sense mutations, deletions, or insertions. For example, the present invention includes antigen-binding proteins which include an immunoglobulin light chain variant comprising the amino acid sequence set forth in SEQ ID NO: 10, 20, 30, 40, 50, or 60 but having one or more of such mutations and/or an immunoglobulin heavy chain variant comprising the amino acid sequence set forth in SEQ ID NO: 2, 18, 22, 38, 42, or 58 but having one or more of such mutations. In an embodiment of the invention, a variant anti-TMPRSS2 antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The invention further provides variant anti-TMPRSS2 antigen-binding proteins, e.g., antibodies or antigen-binding fragments thereof, comprising one or more variant CDRs (e.g., any one or more of CDR-L1, CDR-L2, CDR-L3, CDR-H1, CDR-H2 and/or CDR-H3) that are set forth herein with at least 70%, 75%, 80%, 85%, 90%, 95%, 98%, 99% or 99.9% sequence identity or similarity to, e.g., SEQ ID NO: 4, 6, 8, 12, 14, and/or 16; or 24, 26, 28, 32, 34, and/or 36; or 44, 46, 48, 52, 54, and/or 56.

Embodiments of the present invention also include variant antigen-binding proteins, e.g., anti-TMPRSS2 antibodies and antigen-binding fragments thereof, that comprise immunoglobulin V_{H}s and V_{L}s; or HCs and LCs, which comprise an amino acid sequence having 70% or more (e.g., 80%, 85%, 90%, 95%, 97% or 99%) overall amino acid sequence identity or similarity to the amino acid sequences of the corresponding V_{H}s, V_{L}s, HCs or LCs specifically set forth herein, but wherein the CDR-L1, CDR-L2, CDR-L3, CDR-H1, CDR-H2 and CDR-H3 of such immunoglobulins are not variants and comprise the amino acid sequence set forth in SEQ ID NOs: 4, 6, 8, 12, 14, and 16; or 24, 26, 28, 32, 34, and 36; or 44, 46, 48, 52, 54, and 56, respectively. Thus, in such embodiments, the CDRs within variant antigen-binding proteins are not, themselves, variants.

Conservatively modified variant anti-TMPRSS2 antibodies and antigen-binding fragments thereof are also part of the present invention. A “conservatively modified variant” or a “conservative substitution” refers to a variant wherein there is one or more substitutions of amino acids in a polypeptide with other amino acids having similar characteristics (e.g. charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45.

Function-conservative variants of the anti-TMPRSS2 antibodies and antigen-binding fragments thereof are also part of the present invention. Any of the variants of the anti-TMPRSS2 antibodies and antigen-binding fragments thereof (as discussed herein) may be “function-conservative variants”. Such function-conservative variants may, in some cases, also be characterized as conservatively modified variants. “Function-conservative variants,” as used herein, refers to variants of the anti-TMPRSS2 antibodies or antigen-binding fragments thereof in which one or more amino acid residues have been changed without significantly altering one or more functional properties of the antibody or fragment. In an embodiment of the invention, a function-conservative variant anti-TMPRSS2 antibody or antigen-binding fragment thereof of the present invention comprises a variant amino acid sequence and exhibits one or more of the following functional properties:
Inhibits growth of influenza virus (e.g., A/Puerto Rico/08/1934 (H1N1)) or coronavirus (e.g., SARS-CoV-2) in TMPRSS2-expressing cells (e.g., Calu-3 cells);
Does not significantly bind to MDCK/Tet-on cells which do not express TMPRSS2;
Limits spread of coronavirus infection (e.g., by SARS-CoV-2) or influenza virus infection (e.g., by H1_PR34; H1_CA09; H1_Bris; H9N2 or H3N2 influenza virus) of cells, e.g., Calu-3, in vitro; and/or
Protects a mouse engineered to express the human TMPRSS2 protein from death caused by influenza virus infection (e.g., H1N1, or H3N2) or coronavirus infection (e.g., SARS-CoV-2), for example, wherein the mice are infected with an otherwise lethal dose of the virus, optionally when combined with an anti-HA or an anti-spike protein antibody.
Protects a mouse engineered to express the human TMPRSS2 protein from weight loss caused by influenza virus infection (e.g., H1N1, or H3N2) or coronavirus infection (e.g., SARS-CoV-2), for example, wherein the mice are infected with a dose of the virus that would otherwise cause weight loss, optionally when combined with an anti-HA or an anti-spike protein antibody.

The present invention includes a mouse engineered to express the human TMPRSS2 protein which includes, within the mouse's body, an anti-TMPRSS2 antigen-binding protein (e.g., antibody or antigen-binding fragment) such as mAb8021, mAb8028, or mAb8029. See International patent application publication no. WO2017/151453.

A “neutralizing” or “antagonist” anti-TMPRSS2 antigen-binding protein, e.g., antibody or antigen-binding fragment, refers to a molecule that inhibits an activity of TMPRSS2 to any detectable degree, e.g., inhibits protease activity of TMPRSS2, for example, of a substrate such as HA; Cbz-Gly-Gly-Arg-AMC (Sigma), where Cbz is benzyloxycarbonyl and AMC is 7-amino-4-methylcoumarin; influenza virus HA0; coronavirus S protein; or precursor TMPRSS2 which is autocatalytically cleaved between Arg255 and Ile256 and/or inhibits influenza virus entry into a cell and/or inhibits influenza virus reproduction in the body of a subject.

mAb8021, mAb8028, and mAb8029 refer to antigen-binding proteins, such as antibodies and antigen-binding fragments thereof, that comprise the heavy chain or V_{H} (or a variant thereof) and light chain or V_{L} (or a variant thereof) as set forth below; or that comprise a V_{H} that comprises the CDRs thereof (CDR-H1 (or a variant thereof), CDR-H2 (or a variant thereof) and CDR-H3 (or a variant thereof)) and a V_{L} that comprises the CDRs thereof (CDR-L1 (or a variant thereof), CDR-L2 (or a variant thereof) and CDR-L3 (or a variant thereof)), e.g., wherein the immunoglobulin chains, variable regions and/or CDRs comprise the specific amino acid sequences described below.

In an embodiment of the invention, mAb8028 refers to an antibody or antigen-binding fragment thereof comprising CDR-H1, CDR-H2, and CDR-H3 of an immunoglobulin heavy chain that comprises the amino acid sequence set forth in SEQ ID NO: 2 or 18 and CDR-L1, CDR-L2, and CDR-L3 of an immunoglobulin light chain that comprises the amino acid sequence set forth in SEQ ID NO: 10 or 20.

In an embodiment of the invention, mAb8021 refers to an antibody or antigen-binding fragment thereof comprising CDR-H1, CDR-H2, and CDR-H3 of an immunoglobulin heavy chain that comprises the amino acid sequence set forth in SEQ ID NO: 22 or 38 and CDR-L1, CDR-L2, and CDR-L3 of an immunoglobulin light chain that comprises the amino acid sequence set forth in SEQ ID NO: 30 or 40.

In an embodiment of the invention, mAb8029 refers to an antibody or antigen-binding fragment thereof comprising CDR-H1, CDR-H2, and CDR-H3 of an immunoglobulin heavy chain that comprises the amino acid sequence set forth in SEQ ID NO: 42 or 58 and CDR-L1, CDR-L2, and CDR-L3 of an immunoglobulin light chain that comprises the amino acid sequence set forth in SEQ ID NO: 50 or 60.

In an embodiment of the invention, mAb8028 refers to an antibody or antigen-binding fragment thereof comprising a V_{H} that comprises the amino acid sequence set forth in SEQ ID NO: 2; and a V_{L} that comprises the amino acid sequence set forth in SEQ ID NO: 10.

In an embodiment of the invention, mAb8021 refers to an antibody or antigen-binding fragment thereof comprising a V_{H} that comprises the amino acid sequence set forth in SEQ ID NO: 22; and a V_{L} that comprises the amino acid sequence set forth in SEQ ID NO: 30.

In an embodiment of the invention, mAb8029 refers to an antibody or antigen-binding fragment thereof comprising a V_{H} that comprises the amino acid sequence set forth in SEQ ID NO: 42; and a V_{L} that comprises the amino acid sequence set forth in SEQ ID NO: 50.

In an embodiment of the invention, mAb8028 refers to an antibody or antigen-binding fragment comprising a heavy chain immunoglobulin that comprises the amino acid sequence set forth in SEQ ID NO: 18; and a light chain immunoglobulin that comprises the amino acid sequence set forth in SEQ ID NO: 20.

In an embodiment of the invention, mAb8021 refers to an antibody or antigen-binding fragment comprising a heavy chain immunoglobulin that comprises the amino acid sequence set forth in SEQ ID NO: 38; and a light chain immunoglobulin that comprises the amino acid sequence set forth in SEQ ID NO: 40.

In an embodiment of the invention, mAb8029 refers to an antibody or antigen-binding fragment comprising a heavy chain immunoglobulin that comprises the amino acid sequence set forth in SEQ ID NO: 58; and a light chain immunoglobulin that comprises the amino acid sequence set forth in SEQ ID NO: 60.

The antibodies described herein also include embodiments wherein the V_{H} is fused to a wild-type IgG4 (e.g., wherein residue 108 is S) or to IgG4 variants (e.g., wherein residue 108 is P).

Antibodies and antigen-binding fragments of the present invention comprise immunoglobulin chains including the amino acid sequences set forth herein as well as cellular and in vitro post-translational modifications to the antibody. For example, the present invention includes antibodies and antigen-binding fragments thereof that specifically bind to TMPRSS2 comprising heavy and/or light chain amino acid sequences set forth herein (e.g., CDR-H1, CDR-H2, CDR-H3, CDR-L1, CDR-L2 and/or CDR-L3) as well as antibodies and fragments wherein one or more amino acid residues is glycosylated, one or more Asn residues is deamidated, one or more residues (e.g., Met, Trp and/or His) is oxidized, the N-terminal Gln is pyroglutamate (pyroE) and/or the C-terminal Lysine is missing.

### Administration of Antibodies

The present invention provides methods for administering an anti-TMPRSS2 antigen-binding protein of the present invention, e.g., mAb8021, mAb8028, or mAb8029, comprising introducing the antigen-binding protein into the body of a subject (e.g., a human). For example, the method comprises piercing the body of the subject with a needle of a syringe and injecting the antigen-binding protein into the body of the subject, e.g., into the vein, artery, tumor, muscular tissue or subcutis of the subject.

The present invention provides a vessel (e.g., a plastic or glass vial, e.g., with a cap or a chromatography column, hollow bore needle or a syringe cylinder) comprising an anti-TMPRSS2 antigen-binding protein of the present invention, e.g., mAb8021, mAb8028, or mAb8029.

The present invention also provides an injection device comprising one or more antigen-binding proteins (e.g., antibody or antigen-binding fragment) that bind specifically to TMPRSS2, e.g., mAb8021, mAb8028, or mAb8029, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antigen-binding protein, e.g., an antibody or antigen-binding fragment thereof, from a combination of the present invention, or a pharmaceutical composition thereof is an intravenous (IV) injection device. Such a device can include the antigen-binding protein or a pharmaceutical composition thereof in a cannula or trocar/needle which may be attached to a tube which may be attached to a bag or reservoir for holding fluid (e.g., saline) introduced into the body of the subject through the cannula or trocar/needle. The antibody or fragment or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a subject's body. External infusion pumps are medical devices that deliver the antibody or fragment or a pharmaceutical composition thereof into a subject's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Preparation of Human Antibodies

Methods for generating human antibodies in transgenic mice are known in the art. Any such known methods can be used in the context of the present invention to make human antibodies that specifically bind to TMPRSS2. An immunogen comprising any one of the following can be used to generate antibodies to TMPRSS2. In certain embodiments of the invention, the antibodies of the invention are obtained from mice immunized with a full length, native TMPRSS2, or with a live attenuated or inactivated virus, or with DNA encoding the protein or fragment thereof. Alternatively, the TMPRSS2 protein or a fragment thereof may be produced using standard biochemical techniques and modified and used as immunogen. In one embodiment of the invention, the immunogen is a recombinantly produced TMPRSS2 protein or fragment thereof. In certain embodiments of the invention, the immunogen may be a TMPRSS2 polypeptide vaccine. In certain embodiments, one or more booster injections may be administered. In certain embodiments, the immunogen may be a recombinant TMPRSS2 polypeptide expressed in E. coli or in any other eukaryotic or mammalian cells such as Chinese hamster ovary (CHO) cells.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to TMPRSS2 can be initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

### Anti-TMPRSS2 Antibodies Comprising Fc Variants

According to certain embodiments of the present invention, anti-TMPRSS2 antigen-binding proteins, e.g., antibodies or antigen-binding fragments, are provided comprising an Fc domain comprising one or more mutations, which, for example, enhance or diminish antibody binding to the FcRn receptor, e.g., at acidic pH as compared to neutral pH. For example, the present invention includes anti-TMPRSS2 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-TMPRSS2 antigen-binding proteins, e.g., antibodies or antigen-binding fragments, comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 3111 (e.g., P257I and Q3111); 257I and 434H (e.g., P257I and N434H); 376V and 434H (e.g., D376V and N434H); 307A, 380A and 434A (e.g., T307A, E380A and N434A); and 433K and 434F (e.g., H433K and N434F).

Anti-TMPRSS antigen-binding proteins, e.g., antibodies and antigen-binding fragments thereof, that comprise a V_{H} and/or V_{L} as set forth herein comprising any possible combinations of the foregoing Fc domain mutations, are contemplated within the scope of the present invention.

The present invention also includes anti-TMPRSS2 antigen-binding proteins, antibodies or antigen-binding fragments, comprising a V_{H} set forth herein and a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., WO2014/022540).

### Immunoconjugates

The invention encompasses an anti-TMPRSS2 antigen-binding proteins, e.g., antibodies or antigen-binding fragments, conjugated to another moiety, e.g., a therapeutic moiety (an “immunoconjugate”), such as a toxoid or an anti-viral drug to treat influenza virus infection. In an embodiment of the invention, an anti-TMPRSS2 antibody or fragment is conjugated to any of the further therapeutic agents set forth herein. As used herein, the term “immunoconjugate” refers to an antigen-binding protein, e.g., an antibody or antigen-binding fragment, which is chemically or biologically linked to a radioactive agent, a cytokine, an interferon, a target or reporter moiety, an enzyme, a peptide or protein or a therapeutic agent. The antigen-binding protein may be linked to the radioactive agent, cytokine, interferon, target or reporter moiety, enzyme, peptide or therapeutic agent at any location along the molecule so long as it is able to bind its target (TMPRSS2). Examples of immunoconjugates include antibody-drug conjugates and antibody-toxin fusion proteins. In one embodiment of the invention, the agent may be a second, different antibody that binds specifically to TMPRSS2. The type of therapeutic moiety that may be conjugated to the anti-TMPRSS2 antigen-binding protein (e.g., antibody or fragment) will take into account the condition to be treated and the desired therapeutic effect to be achieved. See, e.g., Amon et al., “Monoclonal Antibodies For Immunotargeting Of Drugs In Cancer Therapy”, Monoclonal Antibodies And Cancer Therapy, Reisfeld et al. (eds.), pp. 243-56 (Alan R. Liss, Inc. 1985); Hellstrom et al., “Antibodies For Drug Delivery”, Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, Monoclonal Antibodies 1984: Biological And Clinical Applications, Pinchera et al. (eds.), pp. 475-506 (1985); “Analysis, Results, And Future Prospective Of The Therapeutic Use Of Radiolabeled Antibody In Cancer Therapy”, Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

### Multi-Specific Antibodies

The present invention includes anti-TMPRSS2 antigen-binding proteins, e.g., antibodies and antigen-binding fragments thereof, as well as methods of use thereof and methods of making such antigen-binding proteins. The term “anti-TMPRSS2” antigen-binding proteins, e.g., antibodies or antigen-binding fragments, includes multispecific (e.g., bispecific or biparatopic) molecules that include at least one first antigen-binding domain that specifically binds to TMPRSS2 (e.g., an antigen-binding domain from mAb8021, mAb8028, or mAb8029) and at least one second antigen-binding domain that binds to a different antigen or to an epitope in TMPRSS2 which is different from that of the first antigen-binding domain (e.g., influenza HA such as an antigen-binding domain from mAb8021, mAb8028, or mAb8029). In an embodiment of the invention, the first and second epitopes overlap. In another embodiment of the invention, the first and second epitopes do not overlap. For example, in an embodiment of the invention, a multispecific antibody is a bispecific IgG antibody (e.g., IgG1 or IgG4) that includes a first antigen-binding domain that binds specifically to TMPRSS2 including the heavy and light immunoglobulin chain of mAb8021, mAb8028, or mAb8029, and a second antigen-binding domain that binds specifically to influenza HA (comprising a different light and heavy immunoglobulin chain such as from mAb8021, mAb8028, or mAb8029).

mAb8021, mAb8028, and mAb8029 include multispecific molecules, e.g., antibodies or antigen-binding fragments, that include the CDR-Hs and CDR-Ls, V_{H} and V_{L}, or HC and LC of mAb8021, mAb8028, or mAb8029, respectively (including variants thereof as set forth herein).

In an embodiment of the invention, an antigen-binding domain that binds specifically to TMPRSS, which may be included in a multispecific molecule, comprises:
(1)
   (i) a heavy chain variable domain sequence that comprises CDR-H1 comprising the amino acid sequence set forth in SEQ ID NO: 4, CDR-H2 comprising the amino acid sequence set forth in SEQ ID NO: 6, and CDR-H3 comprising the amino acid sequence set forth in SEQ ID NO: 8, and
   (ii) a light chain variable domain sequence that comprises CDR-L1 comprising the amino acid sequence set forth in SEQ ID NO: 12, CDR-L2 comprising the amino acid sequence set forth in SEQ ID NO: 14, and CDR-L3 comprising the amino acid sequence set forth in SEQ ID NO: 16;
   or,
(2)
   (i) a heavy chain variable domain sequence comprising the amino acid sequence set forth in SEQ ID NO: 2, and
   (ii) a light chain variable domain sequence comprising the amino acid sequence set forth in SEQ ID NO: 10;
or,
(3)
   (i) a heavy chain immunoglobulin sequence comprising the amino acid sequence set forth in SEQ ID NO: 18, and
   (ii) a light chain immunoglobulin sequence comprising the amino acid sequence set forth in SEQ ID NO: 20.

In an embodiment of the invention, an antigen-binding domain that binds specifically to TMPRSS, which may be included in a multispecific molecule, comprises:
(1)
   (i) a heavy chain variable domain sequence that comprises CDR-H1 comprising the amino acid sequence set forth in SEQ ID NO: 24, CDR-H2 comprising the amino acid sequence set forth in SEQ ID NO: 26, and CDR-H3 comprising the amino acid sequence set forth in SEQ ID NO: 28, and
   (ii) a light chain variable domain sequence that comprises CDR-L1 comprising the amino acid sequence set forth in SEQ ID NO: 32, CDR-L2 comprising the amino acid sequence set forth in SEQ ID NO: 34, and CDR-L3 comprising the amino acid sequence set forth in SEQ ID NO: 36;
or,
(2)
   (i) a heavy chain variable domain sequence comprising the amino acid sequence set forth in SEQ ID NO: 22, and
   (ii) a light chain variable domain sequence comprising the amino acid sequence set forth in SEQ ID NO: 30;
or,
(3)
   (i) a heavy chain immunoglobulin sequence comprising the amino acid sequence set forth in SEQ ID NO: 38, and
   (ii) a light chain immunoglobulin sequence comprising the amino acid sequence set forth in SEQ ID NO: 40.

In an embodiment of the invention, an antigen-binding domain that binds specifically to TMPRSS, which may be included in a multispecific molecule, comprises:
(1)
   (i) a heavy chain variable domain sequence that comprises CDR-H1 comprising the amino acid sequence set forth in SEQ ID NO: 44, CDR-H2 comprising the amino acid sequence set forth in SEQ ID NO: 46, and CDR-H3 comprising the amino acid sequence set forth in SEQ ID NO: 48, and
   (ii) a light chain variable domain sequence that comprises CDR-L1 comprising the amino acid sequence set forth in SEQ ID NO: 52, CDR-L2 comprising the amino acid sequence set forth in SEQ ID NO: 54, and CDR-L3 comprising the amino acid sequence set forth in SEQ ID NO: 56;
or,
(2)
   (i) a heavy chain variable domain sequence comprising the amino acid sequence set forth in SEQ ID NO: 42, and
   (ii) a light chain variable domain sequence comprising the amino acid sequence set forth in SEQ ID NO: 50;
or,
(3)
   (i) a heavy chain immunoglobulin sequence comprising the amino acid sequence set forth in SEQ ID NO: 58, and
   (ii) a light chain immunoglobulin sequence comprising the amino acid sequence set forth in SEQ ID NO: 60.

In an embodiment of the invention, the multispecific antibody or fragment includes more than two different binding specificities (e.g., a trispecific molecule), for example, one or more additional antigen-binding domains which are the same or different from the first and/or second antigen-binding domain.

In an embodiment of the invention, a multispecific molecule comprises, in addition to an antigen-binding site that bind specifically to TMPRSS2, an antigen-binding site that binds specifically to a coronavirus spike protein taken from an antibody selected from the group consisting of: H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2, as set forth in International patent application publication no. WO/2015/179535.

In an embodiment of the invention, a multispecific molecule comprises, in addition to an antigen-binding site that bind specifically to TMPRSS2, an antigen-binding site that binds specifically to influenza HA taken from an antibody selected from the group consisting of:

H1H14611N2; H1H14612N2; H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; and H1H18335B; as set forth in International patent application publication no. WO2016/100807 (e.g., the CDR-Hs, V_{H} or heavy chain thereof; and the CDR-Ls, V_{L} or light chain thereof).

In an embodiment of the invention, a multispecific molecule comprises, in addition to an antigen-binding site that binds specifically to TMPRSS2, an antigen-binding site that binds specifically to influenza Group II HA protein, e.g., which comprises V_{H} and V_{L} of H1H14611N2; or a heavy chain immunoglobulin comprising CDR-H1, CDR-H2 and CDR-H3 of H1H14611N2 and a light chain immunoglobulin comprising CDR-L1, CDR-L2 and CDR-L3 of H1H14611N2.

In an embodiment of the invention, a multispecific molecule comprises, in addition to an antigen-binding site that bind specifically to TMPRSS2, an antigen-binding site that binds specifically to influenza Group II HA protein, e.g., which comprises V_{H} and V_{L} of H1H14612N2; or a heavy chain immunoglobulin comprising CDR-H1, CDR-H2 and CDR-H3 of H1H14612N2 and a light chain immunoglobulin comprising CDR-L1, CDR-L2 and CDR-L3 of H1H14612N2.

In an embodiment of the invention, a multispecific molecule comprises, in addition to an antigen-binding site that bind specifically to TMPRSS2, an antigen-binding site that binds specifically to influenza Group I HA protein, e.g., which comprises V_{H} and V_{L} of H1H11729P; or a heavy chain immunoglobulin comprising CDR-H1, CDR-H2 and CDR-H3 of H1H11729P and a light chain immunoglobulin comprising CDR-L1, CDR-L2 and CDR-L3 of H1H11729P.

In one embodiment of the invention, a bispecific antigen-binding fragment comprises a first scFv (e.g., comprising V_{H} and V_{L} of mAb8021, mAb8028, or mAb8029) having binding specificity for a first epitope (e.g., TMPRSS2) and a second scFv (e.g., comprising V_{H} and V_{L} of an anti-influenza HA antibody) having binding specificity for a second, different epitope. For example, in an embodiment of the invention, the first and second scFv are tethered with a linker, e.g., a peptide linker (e.g., a GS linker such as (GGGGS)n (SEQ ID NO: 62) wherein n is, for example, 1, 2, 3, 4, 5, 6, 7, 8, 9 or 10). Other bispecific antigen-binding fragments include an F(ab)₂ of a bispecific IgG antibody which comprises the heavy and light chain CDRs of mAb8021 or mAb8028 or mAb8029 and of another antibody that binds to a different epitope.

### Therapeutic Methods

The present invention provides methods for treating or preventing viral infection or cancer (e.g., prostate cancer) by administering a therapeutically effective amount of anti-TMPRSS2 antigen-binding protein, e.g., antibody or antigen-binding fragment, (e.g., mAb8021, mAb8028, or mAb8029) to a subject (e.g., a human) in need of such treatment or prevention.

Coronavirus or influenza virus infection may be treated or prevented, in a subject, by administering an anti-TMPRSS2 antigen-binding protein of the present invention to a subject. Influenza viruses are classified into types A, B and C on the basis of their core proteins. The subtypes of influenza A viruses are determined by envelope glycoproteins possessing either hemagglutinin (HA) or neuraminidase (NA) activity. There are several HA subtypes (e.g., HA1, HA2, HA3, HA4, HA5, HA6, HA7, HA8, HA9, HA10, HA11, HA12, HA13, HA14, HA15, HA16, HA17 or HA18—these subtypes may be designated as H1, H2, H3, etc.) and NA subtypes (e.g., NA1, NA2, NA3, NA4, NA5, NA6, NA7, NA8, NA9, NA10 or NA11—these subtypes may be designated as N1, N2, N3, etc.) of influenza A viruses which are used to designate influenza A subtype. For example, Influenza A virus H1N1 and H3N2 are commonly known human pathogens. Humans are commonly infected by viruses of the subtypes H1, H2 or H3, and N1 or N2. The present invention includes methods for treating or preventing infection with an influenza virus subtype discussed herein. Multispecific antibodies and antigen-binding fragments thereof that bind to TMPRSS2, in an embodiment of the invention, also bind to spike protein of a coronavirus (e.g., SARS-CoV-2, MERS-CoV, or SARS-CoV) or to HA and/or to NA of an influenza virus (e.g., an influenza virus of a subtype set forth herein).

An effective or therapeutically effective dose of anti-TMPRSS2 antigen-binding protein, e.g., antibody or antigen-binding fragment (e.g., mAb8021 or mAb8028 or mAb8029), for treating or preventing a viral infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the invention, an effective or therapeutically effective dose of antibody or antigen-binding fragment thereof of the present invention, for treating or preventing viral infection, e.g., in an adult human subject, is about 0.01 to about 200 mg/kg, e.g., up to about 150 mg/kg. In an embodiment of the invention, the dosage is up to about 10.8 or 11 grams (e.g., about 1, 2, 3, 4, 5, 6, 7, 8, 9, 10 or 11 grams). Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

As used herein, the term “subject” refers to a mammal (e.g., rat, mouse, cat, dog, cow, sheep, horse, goat, rabbit), preferably a human, for example, in need of prevention and/or treatment of a disease or disorder such as viral infection or cancer. The subject may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g. subject resides in a densely-populated city or in close proximity to subjects having confirmed or suspected infections of a virus, or choice of employment, e.g. hospital worker, pharmaceutical researcher, traveler to infected area, or frequent flier.

“Treat” or “treating” means to administer an anti-TMPRSS2 antigen-binding protein, e.g., antibody or antigen-binding fragment of the present invention (e.g., mAb8021 or mAb8028 or mAb8029), to a subject having one or more signs or symptoms of a disease or infection, e.g., viral infection, for which the antigen-binding protein is effective when administered to the subject at an effective or therapeutically effective amount or dose (as discussed herein).

The present invention also encompasses prophylactically administering an anti-TMPRSS2 antigen-binding protein, e.g., antibody or antigen-binding fragment thereof of the present invention (e.g., mAb8021 or mAb8028 or mAb8029), to a subject who is at risk of viral infection so as to prevent such infection. Passive antibody-based immunoprophylaxis has proven an effective strategy for preventing subject from viral infection. See e.g., Berry et al., Passive broad-spectrum influenza immunoprophylaxis. Influenza Res Treat. 2014; 2014:267594. Epub 2014 Sep. 22; and Jianqiang et al., Passive immune neutralization strategies for prevention and control of influenza A infections, Immunotherapy. 2012 February; 4(2): 175-186; Prabhu et al., Antivir Ther. 2009; 14(7):911-21, Prophylactic and therapeutic efficacy of a chimeric monoclonal antibody specific for H5 hemagglutinin against lethal H5N1 influenza. “Prevent” or “preventing” means to administer an anti-TMPRSS2 antigen-binding protein, e.g., antibody or antigen-binding fragment of the present invention (e.g., mAb8021 or mAb8028 or mAb8029), to a subject to inhibit the manifestation of a disease or infection (e.g., viral infection) in the body of a subject, for which the antigen-binding protein is effective when administered to the subject at an effective or therapeutically effective amount or dose (as discussed herein).

In an embodiment of the invention, a sign or symptom of a viral infection in a subject is survival or proliferation of virus in the body of the subject, e.g., as determined by viral titer assay (e.g., coronavirus or influenza virus propagation in embryonated chicken eggs, coronavirus spike protein assay, or influenza virus hemagglutination assay). Other signs and symptoms of viral infection are discussed herein.

The present invention provides a method for treating or preventing viral infection (e.g., influenza virus or coronavirus infection) or for inducing the regression or elimination or inhibiting the progression of at least one sign or symptom of viral infection such as:
fever or feeling feverish/chills;
cough;
sore throat;
runny or stuffy nose;
sneezing;
muscle or body aches;
headaches;
fatigue (tiredness);
vomiting;
diarrhea;
respiratory tract infection;
chest discomfort;
shortness of breath;
bronchitis; and/or
pneumonia,
   which sign or symptom is secondary to viral infection, in a subject in need thereof (e.g., a human), by administering a therapeutically effective amount of anti-TMPRSS2 antigen-binding protein (e.g., mAb8021 or mAb8028 or mAb8029) to the subject, for example, by injection of the protein into the body of the subject.

The present invention also includes methods for treating or preventing cancer, e.g., metastatic cancer, e.g., prostate cancer (e.g., which is characterized by expression of a TMPRSS2:ERG fusion), colon cancer, lung cancer, pancreas cancer, urinary tract cancer, breast cancer, ovarian cancer, prostate adenocarcinoma, renal cell carcinoma, colorectal adenocarcinoma, lung adenocarcinoma, lung squamous cell carcinoma and/or pleural mesothelioma, in a subject, by administering a therapeutically effective amount of TMPRSS2 antigen-binding protein (e.g., mAb8021 or mAb8028 or mAb8029) to the subject, for example, by injection of the protein into the body of the subject. In an embodiment of the invention, the subject is also administered the TMPRSS2 antigen-binding protein in association with a further therapeutic agent, for example, an anti-cancer therapeutic agent. In an embodiment of the invention, the cancer is a tumor whose cells express TMPRSS2 or a variant thereof.

### Combinations and Pharmaceutical Compositions

To prepare pharmaceutical compositions of the anti-TMPRSS2 antigen-binding proteins, e.g., antibodies and antigen-binding fragments thereof (e.g., mAb8021 or mAb8028 or mAb8029), antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the invention, the pharmaceutical composition is sterile. Such compositions are part of the present invention.

The scope of the present invention includes desiccated, e.g., freeze-dried, compositions comprising an anti-TMPRSS2 antigen-binding proteins, e.g., antibody or antigen-binding fragment thereof (e.g., mAb8021 or mAb8028 or mAb8029), or a pharmaceutical composition thereof that includes a pharmaceutically acceptable carrier but substantially lacks water.

In a further embodiment of the invention, a further therapeutic agent that is administered to a subject in association with an anti-TMPRSS2 antigen-binding protein, e.g., antibody or antigen-binding fragment thereof (e.g., mAb8021 or mAb8028 or mAb8029), disclosed herein is administered to the subject in accordance with the Physicians' Desk Reference 2003 (Thomson Healthcare; 57^{th} edition (Nov. 1, 2002)).

The mode of administration can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial.

The present invention provides methods for administering an anti-TMPRSS2 antigen-binding protein, e.g., antibody or antigen-binding fragment thereof (e.g., mAb8021 or mAb8028 or mAb8029), comprising introducing the protein into the body of a subject. For example, the method comprises piercing the body of the subject with a needle of a syringe and injecting the antigen-binding protein into the body of the subject, e.g., into the vein, artery, tumor, muscular tissue or subcutis of the subject.

The present invention provides a vessel (e.g., a plastic or glass vial, e.g., with a cap or a chromatography column, hollow bore needle or a syringe cylinder) comprising any of the anti-TMPRSS2 antigen-binding proteins, e.g., antibodies or antigen-binding fragments thereof (e.g., mAb8021, mAb8028 or mAb8029), polypeptides (e.g., an HC, LC, V_{H} or V_{L} of mAb8021, mAb8028, or mAb8029) or polynucleotides or vectors set forth herein or a pharmaceutical composition thereof comprising a pharmaceutically acceptable carrier.

In an embodiment of the invention, an anti-TMPRSS2 antigen-binding protein, e.g., antibody or antigen-binding fragment thereof of the present invention (e.g., mAb8021, mAb8028, or mAb8029), is in association with one or more further therapeutic agents. For example, in an embodiment of the invention, the further therapeutic agent is an anti-viral drug and/or a vaccine. As used herein, the term “anti-viral drug” refers to any anti-infective drug or therapy used to treat, prevent, or ameliorate a viral infection in a subject. The term “anti-viral drug” includes, but is not limited to a cationic steroid antimicrobial, leupeptin, aprotinin, amantadine, rimantadine, oseltamivir, zanamivir, ribavirin, or interferon-alpha2b. Methods for treating or preventing virus (e.g., coronavirus or influenza virus) infection in a subject in need of said treatment or prevention by administering mAb8021, mAb8028, or mAb8029 in association with a further therapeutic agent are part of the present invention.

For example, in an embodiment of the invention, the further therapeutic agent is a vaccine, e.g., a coronavirus vaccine or an influenza vaccine. In an embodiment of the invention, a vaccine is an inactivated/killed virus vaccine, a live attenuated virus vaccine or a virus subunit vaccine.

For example, in an embodiment of the invention, the further therapeutic agent is:

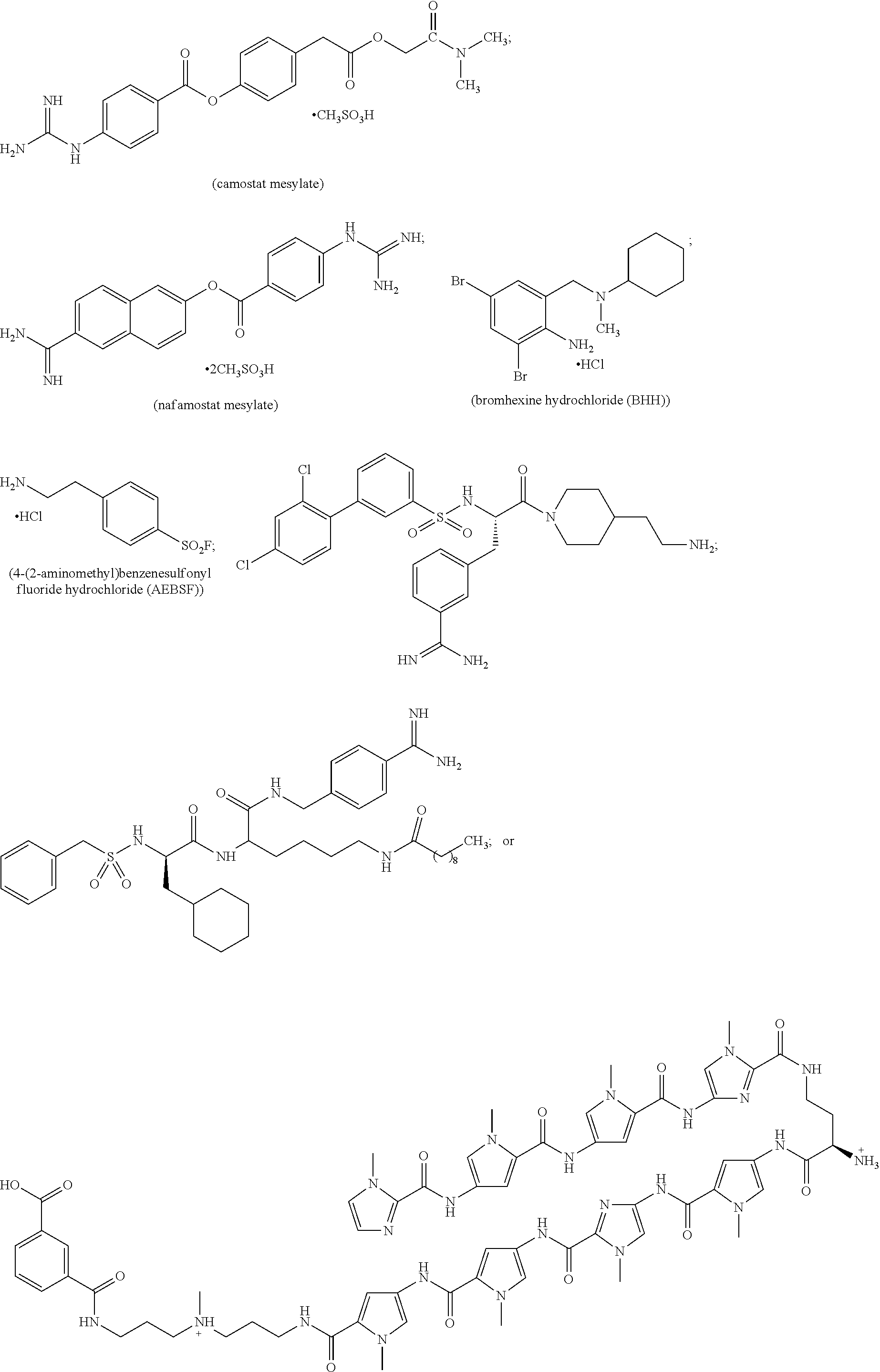

(polyamide). See Shen et al. Biochimie 142: 1-10 (2017).

In an embodiment of the invention, the anti-viral drug is an antibody or antigen-binding fragment that binds specifically to coronavirus, e.g., CoV-S. For example, in an embodiment of the invention, the anti-CoV-S antibody is any one of: H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2, as set forth in International patent application publication no. WO/2015/179535, or an antigen-binding fragment thereof, e.g., wherein the antibody or fragment comprises a light chain immunoglobulin that includes CDR-L1, CDR-L2 and CDR-L3 (e.g., the V_{L} or light chain thereof); and a heavy chain that includes CDR-H1, CDR-H2 and CDR-H3 (e.g., the V_{H} or heavy chain thereof) of any of the foregoing anti-CoV-S antibodies.

In an embodiment of the invention, the anti-viral drug is an antibody or antigen-binding fragment that binds specifically to influenza virus, e.g., influenza HA. For example, in an embodiment of the invention, the anti-HA antibody is any one of H1H14611N2; H1H14612N2; H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; or H1H18335B; as set forth in International patent application publication no. WO2016/100807; or an antigen-binding fragment thereof, e.g., wherein the antibody or fragment comprises a light chain immunoglobulin that includes CDR-L1, CDR-L2 and CDR-L3 (e.g., the V_{L} or light chain thereof); and a heavy chain that includes CDR-H1, CDR-H2 and CDR-H3 (e.g., the V_{H} or heavy chain thereof) of any of the foregoing anti-influenza HA antibodies.

In an embodiment of the invention, a further therapeutic agent is an antibody or antigen-binding fragment that binds to influenza Group II HA protein such as H1H14611N2; or an antibody or fragment that comprises V_{H} and V_{L} of H1H14611N2; or a heavy chain immunoglobulin comprising CDR-H1, CDR-H2 and CDR-H3 of H1H14611N2 and a light chain immunoglobulin comprising CDR-L1, CDR-L2 and CDR-L3 of H1H14611N2. “H1H14611N2” refers to any anti-group II HA antibody comprising such sequences.

In an embodiment of the invention, a further therapeutic agent is an antibody or antigen-binding fragment that binds to influenza Group II HA protein such as H1H14612N2; or an antibody or fragment that comprises V_{H} and V_{L} of H1H14612N2; or a heavy chain immunoglobulin comprising CDR-H1, CDR-H2 and CDR-H3 of H1H14612N2 and a light chain immunoglobulin comprising CDR-L1, CDR-L2 and CDR-L3 of H1H14612N2. “H1H14612N2” refers to any anti-group II HA antibody comprising such sequences.

In an embodiment of the invention, a further therapeutic agent is an antibody or antigen-binding fragment that binds to influenza Group I HA protein such as H1H11729P; or an antibody or fragment that comprises V_{H} and V_{L} of H1H11729P; or a heavy chain immunoglobulin comprising CDR-H1, CDR-H2 and CDR-H3 of H1H11729P and a light chain immunoglobulin comprising CDR-L1, CDR-L2 and CDR-L3 of H1H11729P. “H1H11729P” refers to any anti-group I HA antibody comprising such sequences.

In a certain embodiment of the invention, the further therapeutic agent is not amantadine, rimantadine, oseltamivir, zanamivir, aprotinin, leupeptin, a cationic steroid antimicrobial, an influenza vaccine (e.g., killed, live, attenuated whole virus or subunit vaccine), or an antibody against influenza virus (e.g., an anti-hemagglutinin antibody).

The term “in association with” indicates that the components, an anti-TMPRSS2 antigen-binding protein, e.g., antibody or antigen-binding fragment thereof of the present invention, along with another agent such as oseltamivir, can be formulated into a single composition, e.g., for simultaneous delivery, or formulated separately into two or more compositions (e.g., a kit). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route (e.g., wherein an anti-TMPRSS2 antibody or antigen-binding fragment thereof.

### Kits

Further provided are kits comprising one or more components that include, but are not limited to, an anti-TMPRSS2 antigen-binding protein, e.g., an antibody or antigen-binding fragment as discussed herein (e.g., mAb8021, mAb8028, or mAb8029), in association with one or more additional components including, but not limited to, a further therapeutic agent, as discussed herein. The antigen-binding protein and/or the further therapeutic agent can be formulated as a single composition or separately in two or more compositions, e.g., with a pharmaceutically acceptable carrier, in a pharmaceutical composition.

In one embodiment of the invention, the kit includes an anti-TMPRSS2 antigen-binding protein, e.g., an antibody or antigen-binding fragment thereof of the invention (e.g., mAb8021, mAb8028, or mAb8029), or a pharmaceutical composition thereof in one container (e.g., in a sterile glass or plastic vial) and a further therapeutic agent in another container (e.g., in a sterile glass or plastic vial).

In another embodiment, the kit comprises a combination of the invention, including an anti-TMPRSS2 antigen-binding protein, e.g., antibody or antigen-binding fragment thereof of the invention (e.g., mAb8021, mAb8028, or mAb8029), or pharmaceutical composition thereof in combination with one or more further therapeutic agents formulated together, optionally, in a pharmaceutical composition, in a single, common container.

If the kit includes a pharmaceutical composition for parenteral administration to a subject, the kit can include a device (e.g., an injection device) for performing such administration. For example, the kit can include one or more hypodermic needles or other injection devices as discussed above containing the anti-TMPRSS2 antigen-binding protein, e.g., antibody or antigen-binding fragment thereof of the present invention (e.g., mAb8021, mAb8028, or mAb8029).

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Diagnostic Uses of the Antibodies

The anti-TMPRSS2 antigen-binding proteins, e.g., antibodies or antigen-binding fragments thereof of the present invention (e.g., mAb8021, mAb8028, or mAb8029), may be used to detect and/or measure TMPRSS2 in a sample. Exemplary assays for TMPRSS2 may include, e.g., contacting a sample with an anti-TMPRSS2 antigen-binding protein of the invention, wherein the anti-TMPRSS2 antigen-binding protein is labeled with a detectable label or reporter molecule or used as a capture ligand to selectively isolate TMPRSS2 from samples. The presence of an anti-TMPRSS2 antigen-binding protein complexed with TMPRSS2 indicates the presence of TMRPSS2 in the sample. Alternatively, an unlabeled anti-TMPRSS2 antibody can be used in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure TMPRSS2 in a sample include enzyme-linked immunosorbent assay (ELISA), radioimmunoassay (RIA), and fluorescence-activated cell sorting (FACS). Thus, the present invention includes a method for detecting the presence of TMPRSS2 polypeptide in a sample comprising contacting the sample with an anti-TMPRSS2 antigen-binding protein and detecting the presence of a TMPRSS/anti-TMPRSS2 antigen-binding protein wherein the presence of the complex indicates the presence of TMPRSS2.

The present invention includes cell-based ELISA methods using the anti-TMPRSS2 antigen-binding proteins, e.g., antibodies and antigen-binding fragments thereof of the present invention (e.g., mAb8021, mAb8028, or mAb8029), to detect the presence of TMPRSS2 on a cell. In an embodiment of the invention, the method includes the steps:
(i) contacting cells immobilized to a solid surface (e.g., a microplate) to be tested for the presence of TMPRSS2 with an anti-TMPRSS2 antigen-binding protein of the present invention;
(ii) optionally washing the mixture to remove unbound anti-TMPRSS2 antigen-binding protein;
(iii) contacting the anti-TMPRSS2 antigen-binding protein with a labeled secondary antibody or antigen-binding fragment thereof that binds to the anti-TMPRSS2 antigen-binding protein;
(iv) optionally washing the complex to remove unbound antigen-binding protein; and
(v) detecting the presence of the label on the secondary antibody or fragment, wherein detection of the label indicates that the cells contain TMPRSS2. For example, the present invention includes such cell-based ELISA methods for identifying TMPRSS2⁺ cells in a sample.

An anti-TMPRSS2 antigen-binding protein of the invention (e.g., mAb8021, mAb8028, or mAb8029) may be used in a Western blot or immune-protein blot procedure for detecting the presence of TMPRSS2 or a fragment thereof in a sample. Such a procedure forms part of the present invention and includes the steps of e.g.:
(1) providing a membrane or other solid substrate comprising a sample to be tested for the presence of TMPRSS2, e.g., optionally including the step of transferring proteins from a sample to be tested for the presence of TMPRSS2 (e.g., from a PAGE or SDS-PAGE electrophoretic separation of the proteins in the sample) onto a membrane or other solid substrate using a method known in the art (e.g., semi-dry blotting or tank blotting); and contacting the membrane or other solid substrate to be tested for the presence of TMPRSS2 or a fragment thereof with an anti-TMPRSS2 antigen-binding protein of the invention.

Such a membrane may take the form, for example, of a nitrocellulose or vinyl-based (e.g., polyvinylidene fluoride (PVDF)) membrane to which the proteins to be tested for the presence of TMPRSS2 in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contacting the membrane with the anti-TMPRSS2 antigen-binding protein, the membrane is optionally blocked, e.g., with non-fat dry milk or the like so as to bind non-specific protein binding sites on the membrane.
(2) washing the membrane one or more times to remove unbound anti-TMPRSS2 antigen-binding protein and other unbound substances; and
(3) detecting the bound anti-TMPRSS2 antigen-binding protein.

Detection of the bound antigen-binding protein indicates that the TMPRSS2 protein is present on the membrane or substrate and in the sample. Detection of the bound antigen-binding protein may be by binding the antigen-binding protein with a secondary antibody (an anti-immunoglobulin antibody) which is detectably labeled and, then, detecting the presence of the secondary antibody label.

The anti-TMPRSS2 antigen-binding proteins (e.g., antibodies and antigen-binding fragments (e.g., mAb8021, mAb8028, or mAb8029)) disclosed herein may also be used for immunohistochemistry. Such a method forms part of the present invention and comprises, e.g.,
(1) contacting tissue to be tested for the presence of TMPRSS2 protein with an anti-TMPRSS2 antigen-binding protein of the invention; and
(2) detecting the antigen-binding protein on or in the tissue.

If the antigen-binding protein itself is detectably labeled, it can be detected directly. Alternatively, the antigen-binding protein may be bound by a detectably labeled secondary antibody wherein the label is then detected.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to TMPRSS2

Human antibodies to TMPRSS2 were generated in a VELOCIMMUNE® mouse comprising DNA encoding human immunoglobulin heavy and kappa light chain variable regions. The mice were immunized with vectors expressing TMPRSS2, followed by a booster dose of TMPRSS2. The antibody immune response was monitored by a TMPRSS2-specific immunoassay. Anti-TMPRSS2 antibodies were isolated directly from antigen-positive mouse B cells without fusion to myeloma cells, as described in U.S. Pat. No. 7,582,298, herein specifically incorporated by reference in its entirety. Using this method, fully human anti-TMPRSS2 antibodies (i.e., antibodies possessing human variable domains and human constant domains) were obtained.

Exemplary antibodies described herein are designated mAb8028, mAb8021, and mAb8029. The biological properties of the exemplary antibodies generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions (HCVR and LCVR, respectively) and CDRs (HCDR-1, HCDR-2, HCDR-3, LCDR-1, LCDR-2, and LCDR-3), as well as the heavy chain (HC) and light chain (LC) sequences, of the exemplary anti-TMPRSS2 antibodies. The corresponding nucleic acid sequence identifiers are set forth in Table 2.

TABLE 1
Amino acid sequence identifiers

| | SEQUENCE IDENTIFIER | | | | | | | | | |
|---|---|---|---|---|---|---|---|---|---|---|
| | HCVR | CDR-H1 | CDR-H2 | CDR-H3 | LCVR | CDR-L1 | CDR-L2 | CDR-L3 | HC | LC |
| mAb8028 | 2 | 4 | 6 | 8 | 10 | 12 | 14 | 16 | 18 | 20 |
| mAb8021 | 22 | 24 | 26 | 28 | 30 | 32 | 34 | 36 | 38 | 40 |
| mAb8029 | 42 | 44 | 46 | 48 | 50 | 52 | 54 | 56 | 58 | 60 |

TABLE 2
Nucleic acid sequence identifiers

| | SEQUENCE IDENTIFIER | | | | | | | | | |
|---|---|---|---|---|---|---|---|---|---|---|
| | HCVR | CDR-H1 | CDR-H2 | CDR-H3 | LCVR | CDR-L1 | CDR-L2 | CDR-L3 | HC | LC |
| mAb8028 | 1 | 3 | 5 | 7 | 9 | 11 | 13 | 15 | 17 | 19 |
| mAb8021 | 21 | 23 | 25 | 27 | 29 | 31 | 33 | 35 | 37 | 39 |
| mAb8029 | 41 | 43 | 45 | 47 | 49 | 51 | 53 | 55 | 57 | 59 |

Antibodies disclosed herein have fully human variable regions but can have mouse constant regions (e.g., a mouse IgG1 Fc or a mouse IgG2 Fc (a or b isotype)) or human constant regions (e.g., a human IgG1 Fc or a human IgG4 Fc). As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Tables 1 and 2 will remain the same, and the binding properties to antigen are expected to be identical or substantially similar regardless of the nature of the Fc domain.

### Example 3: In Vitro Multicycle Replication

The ability of the influenza virus, Influenza A A/Puerto Rico/08/1934 (H1_PR34), to replicate in Calu3 cells after treatment with mAb8021, mAb8028, or mAb8029 was assessed.

TABLE 3
Reagents used

| Description | Vendor |
|---|---|
| Calu-3 cells | ATCC |
| EMEM | Gibco |
| Non-essential amino acids | Invitrogen |
| F12 | Gibco |
| 3.5 mL 30% low IgG BSA | Sigma |
| Pen/Strep | Gibco |
| Low IgG BSA | Sigma |
| PBS | Life Technologies |
| Fetal Bovine Serum | Life Technologies |
| Influenza A A/Puerto Rico/08/1934 | ATCC |
| (H1_PR34) | |
| Anti-influenza NP (mouse) | Millipore |

Calu-3 cells were seeded at 40,000 cells/well in a 96-well plate in DMEM:F12 medium with 5% FBS. The next day, influenza virus was diluted to a MOI of 0.01 and antibodies were diluted to 25 μg/mL. The HA antibody was pre-incubated with an influenza virus for one hour at 37° C. in a separate plate. After the pre-incubation period, uninfected Calu-3 cells were treated with either the anti-HA antibody/virus mixture, or with one of the anti-TMPRSS2 antibodies mAb8021, mAb8028, or mAb8029 in combination with the influenza virus. The treated Calu-3 cells were then incubated for one hour. After the hour-long infection, the cells were washed three times with PBS and fresh antibody (anti-HA or anti-TMPRSS2, consistent with the previous antibody) was added along with new medium to each well. Additional antibody was added at 24 and 48 hours post-infection. At 72 hours post-infection, the cells were stained with an anti-NP and imaged on a CTL-ImmunoSpot® S6 Universal Analyzer (Cellular Technology Limited, Cleveland, OH).

Calu-3 is an immortalized human airway epithelial cell line which has been shown to allow multicycle replication of human influenza viruses in the absence of exogenous trypsin (Zeng et al., Highly pathogenic avian influenza H5N1 viruses elicit an attenuated type I interferon response in polarized human bronchial epithelial cells. J Virol. 81:12439-12449 (2007)). In addition, Calu-3 cells have been shown to express TMPRSS2, which is important for testing anti-TMPRSS2 antibodies. Although these antibodies were assayed for their ability to prevent the multicycle replication of influenza PR8 virus, the skilled worker will appreciate that TMPRSS2 is also involved in coronavirus infection and, therefore, that blocking TMPRSS2 would have a similar effect on coronavirus infection. Moreover, blocking TMPRSS2 may be beneficial because it has the potential to inhibit broader viral infectivity than blocking a specific viral protein. As a positive control, an anti-HA neutralizing antibody was used for inhibition of infectivity. As a negative control, an isotype control antibody was used. Imaging of the infected cells was performed on a CTL-ImmunoSpot® S6 Universal Analyzer (Cellular Technology Limited, Cleveland, OH). TMPRSS2 mAbs demonstrated inhibition of influenza infection, as observed by examining the differences in the number of infected cells between the anti-TMPRSS2 mAb-treated cells and isotype control-treated cells. The relative degree of mAb-mediated inhibition of virus replication is reported in Table 4, below.

TABLE 4
Viral inhibition by mAb8021, mAb8028, and mAb8029

| | | Level of |
|---|---|---|
| | mAb | virus |
| mAb | target | inhibition |
| mAb8021 | TMPRSS2 | +++ |
| mAb8028 | TMPRSS2 | +++ |
| mAb8029 | TMPRSS2 | +++ |
| Positive Control | Influenza | ++++ |
| | HA | |
| Negative Control | Isotype | − |
| | control | |

Level of virus inhibition relative to infected, untreated control.
No inhibition (−), no virus positive cells (++++), few virus positive cells (+++).

### Example 3: Binding Kinetics of Anti-TMPRSS2 Antibodies

The equilibrium dissociation constant (K_{D}) for TMPRSS2 binding to different TMPRSS2 monoclonal antibodies (mAbs) was determined with a real-time surface plasmon resonance biosensor using a Biacore T200 instrument. All binding studies were performed in 10 mM HEPES, 150 mM NaCl, and 0.05% v/v Surfactant Tween-20, pH 7.4 (HBS-P) running buffer at 25° C. and 37° C. The Biacore CM5 sensor chip surface was first derivatized by amine coupling with anti-human Fc mAb (REGN2567) to capture different TMPRSS2 mAbs expressed with a C-terminal myc-myc-hexahistidine (MMH) tag. Different concentrations (100-3.7 nM, 3-fold serial dilution) of the ectodomain of human TMPRSS2-myc-myc-His (hTMPRSS2-MMH), Macaca fascicularis TMPRSS2-MMH (mfTMPRSS2-MMH), rat TMPRSS2-MMH (rTMPRSS2-MMH), or mouse TMPRSS2-MMH (mTMPRSS2-MMH), prepared in HBS-P running buffer were injected over the TMPRSS2 mAb captured surface for 150 sec at a flow rate of 30 μL/min and their dissociation in HBS-P running buffer was monitored for 10 minutes. At the end of each cycle, the TMPRSS2 mAb captured surface was regenerated using a 12 sec injection of 20 mM phosphoric acid.

The association rate (kₐ) and dissociation rate (k_{d}) were determined by fitting the real-time binding sensorgrams to a 1:1 binding model with mass transport limitation using Scrubber 2.0c curve-fitting software. Binding dissociation equilibrium constant (K_{D}) and dissociative half-life (t½) were calculated from the kinetic rates as:

$$\\ {K}_{D} ( M ) = \frac{kd}{ka} , and t 1 / 2 ( min ) = \frac{ln ( 2 )}{60 * kd}$$

Binding kinetics parameters for TMPRSS2 binding to different TMPRSS2 mAbs of the invention at 25° C. and 37° C. are shown in Tables 5 through 12. Both mAb8021 and mAb8029 exhibited strong binding to TMPRSS2, and mAb8621 in particular exhibited preferential binding to hTMPRSS2 and mfTMPRSS2 over rTMPRSS2 and mTMPRSS2.

TABLE 5
Binding kinetics of anti-TMPRSS2 mAbs
binding to hTMPRSS2-MMH at 25° C.

| | mAb | 100 nM | | | | |
|---|---|---|---|---|---|---|
| | Capture | hTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control* | 226.2 ± 1.9 | 58.4 | 6.02E ± 06 | 4.09E−04 | 6.80E−11 | 28.2 |
| mAb8021 | 199.4 ± 1.5 | 62.6 | 3.08E ± 06 | 9.19E−05 | 2.99E−11 | 125.7 |
| mAb8029 | 234.6 ± 2 | 64.5 | 2.18E ± 06 | 1.29E−04 | 5.92E−11 | 89.5 |

*Positive control antibody H4H7017N, as described in International Patent Pub. No. WO/2019/147831

TABLE 6
Binding kinetics of anti-TMPRSS2 mAbs
binding to hTMPRSS2-MMH at 37° C.

| | mAb | 100 nM | | | | |
|---|---|---|---|---|---|---|
| | Capture | hTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control* | 144.9 ± 1.3 | 28.5 | 9.93E ± 06 | 2.21E−03 | 2.22E−10 | 5.2 |
| mAb8021 | 134.9 ± 1.1 | 40.1 | 4.17E ± 06 | 2.61E−04 | 6.27E−11 | 44.2 |
| mAb8029 | 147.8 ± 0.5 | 35.9 | 3.12E ± 06 | 6.73E−04 | 2.16E−10 | 17.2 |

*Positive control antibody H4H7017N, as described in International Patent Pub. No. WO/2019/147831

TABLE 7
Binding kinetics of anti-TMPRSS2 mAbs binding
to mfTMPRSS2-MMH at 25° C.

| | mAb | 100 nM | | | | |
|---|---|---|---|---|---|---|
| | Capture | mfTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control* | 231.4 ± 4.4 | 47.2 | 6.60E ± 06 | 8.78E−03 | 1.33E−09 | 1.3 |
| mAb8021 | 201.5 ± 1.9 | 63.4 | 4.48E ± 06 | 1.13E−04 | 2.52E−11 | 102.2 |
| mAb8029 | 233.5 ± 2.2 | 66.7 | 3.20E ± 06 | 1.96E−04 | 6.09E−11 | 59.1 |

*Positive control antibody H4H7017N, as described in International Patent Pub. No. WO/2019/147831

TABLE 8
Binding kinetics of anti-TMPRSS2 mAbs binding
to mfTMPRSS2-MMH at 37° C.

| | mAb | 100 nM | | | | |
|---|---|---|---|---|---|---|
| | Capture | mfMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control* | 144.7 ± 0.8 | 11.8 | 1.08E ± 07 | 3.96E−02 | 3.66E−09 | 0.3 |
| mAb8021 | 134.4 ± 0.6 | 36.7 | 7.71E ± 06 | 1.09E−04 | 1.41E−11 | 106.3 |
| mAb8029 | 146.0 ± 0.2 | 35.3 | 5.17E ± 06 | 3.92E−04 | 7.58E−11 | 29.5 |

*Positive control antibody H4H7017N, as described in International Patent Pub. No. WO/2019/147831

TABLE 9
Binding kinetics of anti-TMPRSS2 mAbs
binding to rTMPRSS2-MMH at 25° C.

| | mAb | 100 nM | | | | |
|---|---|---|---|---|---|---|
| | Capture | rTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control** | 207.6 ± 2.4 | 0.24 | NB* | NB* | NB* | NB* |
| mAb8021 | 300.2 ± 0.9 | −0.31 | NB* | NB* | NB* | NB* |
| mAb8029 | 326.7 ± 1.3 | 41.21 | 1.07E ± 05 | 3.64E−04 | 3.40E−09 | 31.8 |

*No binding was observed under the current experimental conditions
**Positive control antibody H4H7017N, as described in International Patent Pub. No. WO/2019/147831

TABLE 10
Binding kinetics of anti-TMPRSS2 mAbs
binding to rTMPRSS2-MMH at 37° C.

| | mAb | 100 nM | | | | |
|---|---|---|---|---|---|---|
| | Capture | rTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control** | 259.6 ± 0.9 | 0.5 | NB* | NB* | NB* | NB* |
| mAb8021 | 365.6 ± 1 | −0.6 | NB* | NB* | NB* | NB* |
| mAb8029 | 404.1 ± 3.7 | 65.4 | 6.82E ± 04 | 1.72E−03 | 2.51E−08 | 6.7 |

*No binding was observed under the current experimental conditions.
**Positive control antibody H4H7017N, as described in International Patent Pub. No. WO/2019/147831

TABLE 11
Binding kinetics of anti-TMPRSS2 mAbs binding
to mTMPRSS2-MMH at 25° C.

| | mAb | 100 nM | | | | |
|---|---|---|---|---|---|---|
| | Capture | mTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control** | 204.6 ± 1.6 | −0.81 | NB* | NB* | NB* | NB* |
| mAb8021 | 298.6 ± 0.8 | −0.8 | NB* | NB* | NB* | NB* |
| mAb8029 | 324.2 ± 1 | 41.99 | 1.33E ± 05 | 4.42E−04 | 3.33E−09 | 26.2 |

*No binding was observed under the current experimental conditions.
**Positive control antibody H4H7017N, as described in International Patent Pub. No. WO/2019/147831

TABLE 12
Binding kinetics of anti-TMPRSS2 mAbs binding
to mTMPRSS2-MMH at 37° C.

| | mAb | 100 nM | | | | |
|---|---|---|---|---|---|---|
| | Capture | mTMPRSS2- | | | | |
| | Level | MMH Bound | kₐ | k_{d} | K_{D} | t½ |
| mAb Captured | (RU) | (RU) | (1/Ms) | (1/s) | (M) | (min) |
| Positive control** | 258.4 ± 2.4 | 0.0 | NB* | NB* | NB* | NB* |
| mAb8021 | 363.0 ± 1.0 | −0.5 | NB* | NB* | NB* | NB* |
| mAb8029 | 402.4 ± 3.3 | 64.7 | 2.14E ± 05 | 2.09E−03 | 9.75E−09 | 5.5 |

*indicates that no binding was observed under the current experimental conditions.
**Positive control antibody H4H7017N, as described in International Patent Pub. No. WO/2019/147831

### Example 4: pH Sensitivity of Anti-TMPRSS2 Antibodies

Dissociation rate constant (k_{d}) for TMPRSS2 binding to purified anti-TMPRSS2 monoclonal antibody was determined using a real-time surface plasmon resonance based Biacore T200 biosensor platform. All binding studies were performed at 37° C. using two running buffers, (i) 1.9 mM NaH2PO4, 8.1 mM Na2HPO4, 2.7 mM KCl, 137 mM NaCl, 0.05% v/v Surfactant Tween-20, pH7.4 (PBS-T-pH7.4), and (ii) 8.8 mM NaH2PO4, 1.2 mM Na2HPO4, 2.7 mM KCl, 137 mM NaCl, 0.05% v/v Surfactant Tween-20, pH6.0 (PBS-T-pH6.0). The CM5 Biacore sensor surface derivatized by amine coupling with an anti-human Fc specific mouse mAb was used to capture different anti-TMPRSS2 mAbs. All the TMPRSS2 reagents were expressed with a C-terminal myc-myc-hexahistidine tag (subsequently referred to as TMPRSS2-MMH). Different concentrations of human TMPRSS2-MMH, Macaca fascicularis TMPRSS2-MMH, rat TMPRSS2-MMH, or mouse TMPRSS2-MMH prepared in PBS-T-pH7.4 running buffer (25 nM-6.25 nM; 4-fold serial dilution) were injected for 2.5 minutes at a flow rate of 30 L/minute followed by the dissociation of bound TMPRSS2-MMH proteins in PBS-T-pH7.4 or PBS-T-pH6.0 running buffers for 8 minutes.

Dissociation rate constants (k_{d}) in two running buffers were determined by fitting the real-time binding sensorgrams to a 1:1 binding model using Scrubber 2.0c curve-fitting software.

Values of dissociation rate for anti-TMPRSS2 mAb binding to human TMPRSS2-MMH, monkey TMPRSS2-MMH, rat TMPRSS2-MMH and mouse TMPRSS2-MMH at 37° C. in PBS-T-pH7.4 and PBS-T-pH6.0 are shown in Table 13 through Table 16. None of the antibodies exhibited pH sensitivity.

TABLE 13
Dissociation rate constants of anti-TMPRSS2 mAbs binding to human TMPRSS2-
MMH at 37° C. in PBS-T-pH 7.4 and PBS-T-pH 6.0

| | Human TMPRSS2-MMH Binding at 37° C. | | | | | | | | |
|---|---|---|---|---|---|---|---|---|---|
| | pH 7.4 Running Buffer | | | | pH 6.0 Running Buffer | | | | |
| | mAb | 100 nm | | | mAb | 100 nm | | | t½ |
| | Capture | Ag | | | Capture | Ag | | | Ratio |
| | Level | Bound | kd | t½ | Level | Bound | kd | t½ | (pH 7.4/ |
| mAb Captured | (RU) | (RU) | (1/s) | (min) | (RU) | (RU) | (1/s) | (min) | pH 6.0) |
| Positive control* | 332 | 66 | 2.63E−03 | 4 | 320.4 | 67 | 3.05E−03 | 4 | 1.2 |
| mAb8021 | 254.9 | 69 | 1.92E−04 | 60 | 245.9 | 71 | 1.29E−04 | 90 | 0.7 |
| mAb8029 | 368.8 | 82 | 6.24E−04 | 19 | 356.2 | 80 | 6.15E−04 | 19 | 1.0 |

*Positive control antibody H4H7017N, as described in International Patent Pub. No. WO/2019/147831

TABLE 14
Dissociation rate constants of anti-TMPRSS2 mAbs binding to monkey
TMPRSS2-MMH at 37° C. in PBS-T-pH 7.4 and PBS-T-pH 6.0

| | Monkey TMPRSS2-MMH Binding at 37° C. | | | | | | | | |
|---|---|---|---|---|---|---|---|---|---|
| | pH 7.4 Running Buffer | | | | pH 6.0 Running Buffer | | | | |
| | mAb | 100 nm | | | mAb | 100 nm | | | t½ |
| | Capture | Ag | | | Capture | Ag | | | Ratio |
| | Level | Bound | kd | t½ | Level | Bound | kd | t½ | (pH 7.4/ |
| mAb Captured | (RU) | (RU) | (1/s) | (min) | (RU) | (RU) | (1/s) | (min) | pH 6.0) |
| Positive control* | 334.7 | 20 | 3.70E−02 | 0.3 | 320.6 | 13 | 4.31E−02 | 0.3 | 1.2 |
| mAb8021 | 253.3 | 71 | 1.27E−04 | 91 | 244.2 | 70 | 1.11E−04 | 104 | 0.9 |
| mAb8029 | 366 | 90 | 4.08E−04 | 28 | 360.1 | 94 | 3.39E−04 | 34 | 0.8 |

*Positive control antibody H4H7017N, as described in International Patent Pub. No. WO/2019/147831

TABLE 15
Dissociation rate constants of anti-TMPRSS2 mAbs binding to rat TMPRSS2-
MMH at 37° C. in PBS-T-pH 7.4 and PBS-T-pH 6.0

| | Rat TMPRSS2-MMH Binding at 37° C. | | | | | | | | |
|---|---|---|---|---|---|---|---|---|---|
| | pH 7.4 Running Buffer | | | | pH 6.0 Running Buffer | | | | |
| | mAb | 100 nm | | | mAb | 100 nm | | | t½ |
| | Capture | Ag | | | Capture | Ag | | | Ratio |
| mAb | Level | Bound | kd | t½ | Level | Bound | kd | t½ | (pH 7.4/ |
| Captured | (RU) | (RU) | (1/s) | (min) | (RU) | (RU) | (1/s) | (min) | pH 6.0) |
| Positive | 297.2 | 0 | NB* | NB* | 295.7 | 0 | NB* | NB* | NB* |
| control** | | | | | | | | | |
| mAb8021 | 333.6 | −1 | NB* | NB* | 332.4 | −1 | NB* | NB* | NB* |
| mAb8029 | 340.4 | 70 | 2.02E−03 | 6 | 338.2 | 71 | 2.28E−03 | 5 | 1.1 |

*No binding was observed under the current experimental conditions
**Positive control antibody H4H7017N, as described in International Patent Pub. No. WO/2019/147831

TABLE 16
Dissociation rate constants of anti-TMPRSS2 mAbs binding to mouse TMPRSS2-
MMH at 37° C. in PBS-T-pH 7.4 and PBS-T-pH 6.0

| | Mouse TMPRSS2-MMH Binding at 37° C. | | | | | | | | |
|---|---|---|---|---|---|---|---|---|---|
| | pH 7.4 Running Buffer | | | | pH 6.0 Running Buffer | | | | |
| | mAb | 100 nm | | | mAb | 100 nm | | | t½ |
| | Capture | Ag | | | Capture | Ag | | | Ratio |
| mAb | Level | Bound | kd | t½ | Level | Bound | kd | t½ | (pH 7.4/ |
| Captured | (RU) | (RU) | (1/s) | (min) | (RU) | (RU) | (1/s) | (min) | pH 6.0) |
| Positive | 330.7 | −1 | NB* | NB* | 317.3 | −1 | NB* | NB* | NB* |
| control** | | | | | | | | | |
| mAb8021 | 251.3 | −2 | NB* | NB* | 244.8 | −1 | NB* | NB* | NB* |
| mAb8029 | 359.9 | 74 | 2.46E−03 | 5 | 360.9 | 74 | 2.57E−03 | 4 | 1.0 |

*No binding was observed under the current experimental conditions.
**Positive control antibody H4H7017N, as described in International Patent Pub. No. WO/2019/147831

### Example 5: Octet Cross-Competition Between Anti-TMPRSS2 Monoclonal Antibodies

Binding competition between TMPRSS2 monoclonal antibodies (mAbs) was determined using a real time, label-free bio-layer interferometry (BLI) assay on the Octet HTX biosensor platform (Pall ForteBio Corp.). The entire experiment was performed at 25° C. in 10 mM HEPES, 150 mM NaCl, 0.05% v/v Surfactant Tween-20, 1 mg/mL BSA, 0.02% NaN3, pH7.4 (HBS-P) buffer with the plate shaking at a speed of 1000 rpm. To assess whether two mAbs are able to compete with one another for binding to their respective epitopes on TMPRSS2, the ectodomain of human TMPRSS2-MMH (hTMPRSS2-MMH) was first captured onto anti-penta-His antibody (HIS1K) coated Octet biosensor tips by submerging the biosensor tips for 1.5 minute in wells containing 10 μg/mL solution of hTMPRSS2-MMH. The hTMPRSS2-MMH captured biosensor tips were then saturated with the first TMPRSS2 mAb (subsequently referred to as mAb-1) by dipping into wells containing 50 g/mL solution of mAb-1 for 5 minutes. The biosensor tips were then subsequently dipped into wells containing 50 g/mL solution of second TMPRSS2 mAb (subsequently referred to as mAb-2) for 5 minutes. The biosensor tips were washed in HBS-P buffer in between every step of the experiment. The real-time binding response was monitored during the entire course of the experiment and the binding response at the end of every step was recorded. The response of mAb-2 binding to hTMPRSS2-MMH pre-complexed with mAb-1 was compared and competitive/non-competitive behavior of different anti-TMPRSS2 mAbs was determined as shown in Table 17.

Dissociation rate constants (k_{d}) in two running buffers were determined by fitting the real-time binding sensorgrams to a 1:1 binding model using Scrubber 2.0c curve-fitting software.

TABLE 17
Cross-competition between anti-TMPRSS2 mAbs

| | mAb-2 Competing |
|---|---|
| mAb-1 | with mAb-1 |
| Positive | mAb8021 |
| control* | mAb8029 |
| mAb8021 | Positive control* |
| | mAb8029 |
| mAb8029 | Positive control* |
| | mAb8021 |

*Positive control antibody H4H7017N, as described in International Patent Pub. No. WO/2019/147831

TABLE 18
Informal Sequence Listing

| SEQ | | |
|---|---|---|
| ID | | |
| NO. | Sequence | Description |
| 1 | gaggtgcagc tggtggagtc tgggggaggc ttggtacagc ctggggggtc cctgagactc | DNA |
| | tcctgtgaag cctctggatt caccttcagg agctacgaca tgcactgggt ccgccaagct | nucleotide |
| | acaggaaaag gtctggagtg ggtctcagct attggttctg ctggtgacac actctatcca | sequence |
| | gactccgtga agggccgatt caccatctcc agagagaatg ccaagaactc cttgtatctt | |
| | caaatggaca gcctgagagc cggggacacg gctgtgtatt actgtgcaag agttggagac | |
| | tggggatcgg ggtacttaga ctactggggc cagggaaccc tggtcaccgt ctcctca | |
| 2 | EVQLVESGGG LVQPGGSLRL SCEASGFTFR SYDMHWVRQA | AA amino |
| | TGKGLEWVSA IGSAGDTLYP DSVKGRFTIS RENAKNSLYL | acid |
| | QMDSLRAGDT AVYYCARVGD WGSGYLDYWG QGTLVTVSS | sequence |
| 3 | ggattcacct tcaggagcta cgac | DNA |
| | | nucleotide |
| | | sequence |
| 4 | GFTFRSYD | AA amino |
| | | acid |
| | | sequence |
| 5 | attggttctg ctggtgacac a | DNA |
| | | nucleotide |
| | | sequence |
| 6 | IGSAGDT | AA amino |
| | | acid |
| | | sequence |
| 7 | gcaagagttg gagactgggg atcggggtac ttagactac | DNA |
| | | nucleotide |
| | | sequence |
| 8 | ARVGDWGSGY LDY | AA amino |
| | | acid |
| | | sequence |
| 9 | gacatccaga tgacccagtc tccatcctcc ctgtctgcat ctgtaggaga cagagtcacc | DNA |
| | atcacttgcc gggcaagtca gagcattagc atctatttaa attggtatca gcagaaacca | nucleotide |
| | gggagagccc ctaaactcct gatctatgtt gcatccagtt tgcaaagtgg ggtcccatca | sequence |
| | aggttcagtg gcagtggatc tgggacagat ttcactctca ccatcagcag tctgcaacct | |
| | gaagattttg caacttacta ctgtcaacag agttacggta ccccattcac tttcggccct | |
| | gggaccaaag tggatatcaa a | |
| 10 | DIQMTQSPSS LSASVGDRVT ITCRASQSIS IYLNWYQQKP | AA amino |
| | GRAPKLLIYV ASSLQSGVPS RFSGSGSGTD FTLTISSLQP | acid |
| | EDFATYYCQQ SYGTPFTFGP GTKVDIK | sequence |
| 11 | cagagcatta gcatctat | DNA |
| | | nucleotide |
| | | sequence |
| 12 | QSISIY | AA amino |
| | | acid |
| | | sequence |
| 13 | gttgcatcc | DNA |
| | | nucleotide |
| | | sequence |
| 14 | VAS | AA amino |
| | | acid |
| | | sequence |
| 15 | caacagagtt acggtacccc attcact | DNA |
| | | nucleotide |
| | | sequence |
| 16 | QQSYGTPFT | AA amino |
| | | acid |
| | | sequence |
| 17 | gaggtgcagc tggtggagtc tgggggaggc ttggtacagc ctggggggtc cctgagactc | DNA |
| | tcctgtgaag cctctggatt caccttcagg agctacgaca tgcactgggt ccgccaagct | nucleotide |
| | acaggaaaag gtctggagtg ggtctcagct attggttctg ctggtgacac actctatcca | sequence |
| | gactccgtga agggccgatt caccatctcc agagagaatg ccaagaactc cttgtatctt | |
| | caaatggaca gcctgagagc cggggacacg gctgtgtatt actgtgcaag agttggagac | |
| | tggggatcgg ggtacttaga ctactggggc cagggaaccc tggtcaccgt ctcctcagcc | |
| | tccaccaagg gcccatcggt cttccccctg gcgccctgct ccaggagcac ctccgagagc | |
| | acagccgccc tgggctgcct ggtcaaggac tacttccccg aaccggtgac ggtgtcgtgg | |
| | aactcaggcg ccctgaccag cggcgtgcac accttcccgg ctgtcctaca gtcctcagga | |
| | ctctactccc tcagcagcgt ggtgaccgtg ccctccagca gcttgggcac gaagacctac | |
| | acctgcaacg tagatcacaa gcccagcaac accaaggtgg acaagagagt tgagtccaaa | |
| | tatggtcccc catgcccacc ctgcccagca cctgagttcc tggggggacc atcagtcttc | |
| | ctgttccccc caaaacccaa ggacactctc atgatctccc ggacccctga ggtcacgtgc | |
| | gtggtggtgg acgtgagcca ggaagacccc gaggtccagt tcaactggta cgtggatggc | |
| | gtggaggtgc ataatgccaa gacaaagccg cgggaggagc agttcaacag cacgtaccgt | |
| | gtggtcagcg tcctcaccgt cctgcaccag gactggctga acggcaagga gtacaagtgc | |
| | aaggtctcca acaaaggcct cccgtcctcc atcgagaaaa ccatctccaa agccaaaggg | |
| | cagccccgag agccacaggt gtacaccctg cccccatccc aggaggagat gaccaagaac | |
| | caggtcagcc tgacctgcct ggtcaaaggc ttctacccca gcgacatcgc cgtggagtgg | |
| | gagagcaatg ggcagccgga gaacaactac aagaccacgc ctcccgtgct ggactccgac | |
| | ggctccttct tcctctacag caggctcacc gtggacaaga gcaggtggca ggaggggaat | |
| | gtcttctcat gctccgtgat gcatgaggct ctgcacaacc actacacaca gaagtccctc | |
| | tccctgtctc tgggtaaatg a | |
| 18 | EVQLVESGGG LVQPGGSLRL SCEASGFTFR SYDMHWVRQA | AA amino |
| | TGKGLEWVSA IGSAGDTLYP | acid |
| | DSVKGRFTIS RENAKNSLYL QMDSLRAGDT AVYYCARVGD | sequence |
| | WGSGYLDYWG QGTLVTVSSA | |
| | STKGPSVFPL APCSRSTSES TAALGCLVKD YFPEPVTVSW | |
| | NSGALTSGVH TFPAVLQSSG | |
| | LYSLSSVVTV PSSSLGTKTY TCNVDHKPSN TKVDKRVESK | |
| | YGPPCPPCPA PEFLGGPSVF | |
| | LFPPKPKDTL MISRTPEVTC VVVDVSQEDP EVQFNWYVDG | |
| | VEVHNAKTKP REEQFNSTYR | |
| | VVSVLTVLHQ DWLNGKEYKC KVSNKGLPSS IEKTISKAKG | |
| | QPREPQVYTL PPSQEEMTKN | |
| | QVSLTCLVKG FYPSDIAVEW ESNGQPENNY KTTPPVLDSD | |
| | GSFFLYSRLT VDKSRWQEGN | |
| | VFSCSVMHEA LHNHYTQKSL SLSLGK | |
| 19 | gacatccaga tgacccagtc tccatcctcc ctgtctgcat ctgtaggaga cagagtcacc | DNA |
| | atcacttgcc gggcaagtca gagcattagc atctatttaa attggtatca gcagaaacca | nucleotide |
| | gggagagccc ctaaactcct gatctatgtt gcatccagtt tgcaaagtgg ggtcccatca | sequence |
| | aggttcagtg gcagtggatc tgggacagat ttcactctca ccatcagcag tctgcaacct | |
| | gaagattttg caacttacta ctgtcaacag agttacggta ccccattcac tttcggccct | |
| | gggaccaaag tggatatcaa acgaactgtg gctgcaccat ctgtcttcat cttcccgcca | |
| | tctgatgagc agttgaaatc tggaactgcc tctgttgtgt gcctgctgaa taacttctat | |
| | cccagagagg ccaaagtaca gtggaaggtg gataacgccc tccaatcggg taactcccag | |
| | gagagtgtca cagagcagga cagcaaggac agcacctaca gcctcagcag caccctgacg | |
| | ctgagcaaag cagactacga gaaacacaaa gtctacgcct gcgaagtcac ccatcagggc | |
| | ctgagctcgc ccgtcacaaa gagcttcaac aggggagagt gttag | |
| 20 | DIQMTQSPSS LSASVGDRVT ITCRASQSIS IYLNWYQQKP | AA amino |
| | GRAPKLLIYV ASSLQSGVPS | acid |
| | RFSGSGSGTD FTLTISSLQP EDFATYYCQQ SYGTPFTFGP | sequence |
| | GTKVDIKRTV AAPSVFIFPP | |
| | SDEQLKSGTA SVVCLLNNFY PREAKVQWKV DNALQSGNSQ | |
| | ESVTEQDSKD STYSLSSTLT | |
| | LSKADYEKHK VYACEVTHQG LSSPVTKSFN RGEC | |
| 21 | caggttcagc tggtgcagtc tggagctgag gtgaagaagc ctggggcctc agtgagggtc | DNA |
| | tcctgcaagg cttctggtta cacctttacc aattatggta tcacctgggt gcgacaggcc | nucleotide |
| | cctggacaag ggcttgagtg gatgggaggg atcagcgttt acaatggtaa cacagaatat | sequence |
| | gcagagaaat tccagggcag agtctccatg accacagaca catccacgac cacagcctac | |
| | ttggagctga ggagcctgaa atctgacgac acggccgtct atttctgtgc gagagggcaa | |
| | ctggactact tcgacccctg gggccaggga accctggtca ctgtctcctc a | |
| 22 | QVQLVQSGAE VKKPGASVRV SCKASGYTFT NYGITWVRQA | AA amino |
| | PGQGLEWMGG ISVYNGNTEY | acid |
| | AEKFQGRVSM TTDTSTTTAY LELRSLKSDD TAVYFCARGQ | sequence |
| | LDYFDPWGQG TLVTVSS | |
| 23 | ggttacacct ttaccaatta tggt | DNA |
| | | nucleotide |
| | | sequence |
| 24 | GYTFTNYG | AA amino |
| | | acid |
| | | sequence |
| 25 | atcagcgttt acaatggtaa caca | DNA |
| | | nucleotide |
| | | sequence |
| 26 | ISVYNGNT | AA amino |
| | | acid |
| | | sequence |
| 27 | gcgagagggc aactggacta cttcgacccc | DNA |
| | | nucleotide |
| | | sequence |
| 28 | ARGQLDYFDP | AA amino |
| | | acid |
| | | sequence |
| 29 | gaaattgtga tgacgcagtc tccagccacc ctgtctgtgt ctccaggaga aagagccacc | DNA |
| | ctctcctgca gggccagtca gagtgttaga agcaacttag cctggtacca gcagaaacct | nucleotide |
| | ggccaggctc ccagtctcct catctatggt gcatccacca gggccactgg tatcccagcc | sequence |
| | aggttcagtg gcagtgggtc tgggacagag ttcactctca ccatcagcag cctgcagtct | |
| | gaagattttg cagtttattt ctgtctgcag tataataact ggtggacgtt cggccaaggg | |
| | accaaggtgg aaatcaaa | |
| 30 | EIVMTQSPAT LSVSPGERAT LSCRASQSVR SNLAWYQQKP | AA amino |
| | GQAPSLLIYG ASTRATGIPA | acid |
| | RFSGSGSGTE FTLTISSLQS EDFAVYFCLQ YNNWWTFGQG | sequence |
| | TKVEIK | |
| 31 | cagagtgtta gaagcaac | DNA |
| | | nucleotide |
| | | sequence |
| 32 | QSVRSN | AA amino |
| | | acid |
| | | sequence |
| 33 | ggtgcatcc | DNA |
| | | nucleotide |
| | | sequence |
| 34 | GAS | AA amino |
| | | acid |
| | | sequence |
| 35 | ctgcagtata ataactggtg gacg | DNA |
| | | nucleotide |
| | | sequence |
| 36 | LQYNNWWT | AA amino |
| | | acid |
| | | sequence |
| 37 | caggttcagc tggtgcagtc tggagctgag gtgaagaagc ctggggcctc agtgagggtc | DNA |
| | tcctgcaagg cttctggtta cacctttacc aattatggta tcacctgggt gcgacaggcc | nucleotide |
| | cctggacaag ggcttgagtg gatgggaggg atcagcgttt acaatggtaa cacagaatat | sequence |
| | gcagagaaat tccagggcag agtctccatg accacagaca catccacgac cacagcctac | |
| | ttggagctga ggagcctgaa atctgacgac acggccgtct atttctgtgc gagagggcaa | |
| | ctggactact tcgacccctg gggccaggga accctggtca ctgtctcctc agcctccacc | |
| | aagggcccat cggtcttccc cctggcgccc tgctccagga gcacctccga gagcacagcc | |
| | gccctgggct gcctggtcaa ggactacttc cccgaaccgg tgacggtgtc gtggaactca | |
| | ggcgccctga ccagcggcgt gcacaccttc ccggctgtcc tacagtcctc aggactctac | |
| | tccctcagca gcgtggtgac cgtgccctcc agcagcttgg gcacgaagac ctacacctgc | |
| | aacgtagatc acaagcccag caacaccaag gtggacaaga gagttgagtc caaatatggt | |
| | cccccatgcc caccctgccc agcacctgag ttcctggggg gaccatcagt cttcctgttc | |
| | cccccaaaac ccaaggacac tctcatgatc tcccggaccc ctgaggtcac gtgcgtggtg | |
| | gtggacgtga gccaggaaga ccccgaggtc cagttcaact ggtacgtgga tggcgtggag | |
| | gtgcataatg ccaagacaaa gccgcgggag gagcagttca acagcacgta ccgtgtggtc | |
| | agcgtcctca ccgtcctgca ccaggactgg ctgaacggca aggagtacaa gtgcaaggtc | |
| | tccaacaaag gcctcccgtc ctccatcgag aaaaccatct ccaaagccaa agggcagccc | |
| | cgagagccac aggtgtacac cctgccccca tcccaggagg agatgaccaa gaaccaggtc | |
| | agcctgacct gcctggtcaa aggcttctac cccagcgaca tcgccgtgga gtgggagagc | |
| | aatgggcagc cggagaacaa ctacaagacc acgcctcccg tgctggactc cgacggctcc | |
| | ttcttcctct acagcaggct caccgtggac aagagcaggt ggcaggaggg gaatgtcttc | |
| | tcatgctccg tgatgcatga ggctctgcac aaccactaca cacagaagtc cctctccctg | |
| | tctctgggta aatga | |
| 38 | QVQLVQSGAE VKKPGASVRV SCKASGYTFT NYGITWVRQA | AA amino |
| | PGQGLEWMGG ISVYNGNTEY | acid |
| | AEKFQGRVSM TTDTSTTTAY LELRSLKSDD TAVYFCARGQ | sequence |
| | LDYFDPWGQG TLVTVSSAST | |
| | KGPSVFPLAP CSRSTSESTA ALGCLVKDYF PEPVTVSWNS | |
| | GALTSGVHTF PAVLQSSGLY | |
| | SLSSVVTVPS SSLGTKTYTC NVDHKPSNTK VDKRVESKYG | |
| | PPCPPCPAPE FLGGPSVFLF | |
| | PPKPKDTLMI SRTPEVTCVV VDVSQEDPEV QFNWYVDGVE | |
| | VHNAKTKPRE EQFNSTYRVV | |
| | SVLTVLHQDW LNGKEYKCKV SNKGLPSSIE KTISKAKGQP | |
| | REPQVYTLPP SQEEMTKNQV | |
| | SLTCLVKGFY PSDIAVEWES NGQPENNYKT TPPVLDSDGS | |
| | FFLYSRLTVD KSRWQEGNVF | |
| | SCSVMHEALH NHYTQKSLSL SLGK | |
| 39 | gaaattgtga tgacgcagtc tccagccacc ctgtctgtgt ctccaggaga aagagccacc | DNA |
| | ctctcctgca gggccagtca gagtgttaga agcaacttag cctggtacca gcagaaacct | nucleotide |
| | ggccaggctc ccagtctcct catctatggt gcatccacca gggccactgg tatcccagcc | sequence |
| | aggttcagtg gcagtgggtc tgggacagag ttcactctca ccatcagcag cctgcagtct | |
| | gaagattttg cagtttattt ctgtctgcag tataataact ggtggacgtt cggccaaggg | |
| | accaaggtgg aaatcaaacg aactgtggct gcaccatctg tcttcatctt cccgccatct | |
| | gatgagcagt tgaaatctgg aactgcctct gttgtgtgcc tgctgaataa cttctatccc | |
| | agagaggcca aagtacagtg gaaggtggat aacgccctcc aatcgggtaa ctcccaggag | |
| | agtgtcacag agcaggacag caaggacagc acctacagcc tcagcagcac cctgacgctg | |
| | agcaaagcag actacgagaa acacaaagtc tacgcctgcg aagtcaccca tcagggcctg | |
| | agctcgcccg tcacaaagag cttcaacagg ggagagtgtt ag | |
| 40 | EIVMTQSPAT LSVSPGERAT LSCRASQSVR SNLAWYQQKP | AA amino |
| | GQAPSLLIYG ASTRATGIPA | acid |
| | RFSGSGSGTE FTLTISSLQS EDFAVYFCLQ YNNWWTFGQG | sequence |
| | TKVEIKRTVA APSVFIFPPS | |
| | DEQLKSGTAS VVCLLNNFYP REAKVQWKVD NALQSGNSQE | |
| | SVTEQDSKDS TYSLSSTLTL | |
| | SKADYEKHKV YACEVTHQGL SSPVTKSFNR GEC | |
| 41 | cagctgcagc tgcaggagtc gggcccagga ctggtgaagc cttcggagac cctgtccctc | DNA |
| | acctgcactg tctctggtgg ctccatcaac agtaatagtt actactgggg ctggatccgc | nucleotide |
| | cagcccccag ggaagggact ggagtggatt gggagtatct attatgatag gaacacctac | sequence |
| | tacaccccgt ccctcaagag tcgagtcacc atatccgtag acacgtccaa gaaccagttc | |
| | tccctgaagc tgacctctgt gaccgccgca gacacggctg tatattattg tgcgagagag | |
| | caggtccccg gctactacgg tatggacgtc tggggccaag ggaccacggt caccgtctcc | |
| | tca | |
| 42 | QLQLQESGPG LVKPSETLSL TCTVSGGSIN SNSYYWGWIR | AA amino |
| | QPPGKGLEWI GSIYYDRNTY | acid |
| | YTPSLKSRVT ISVDTSKNQF SLKLTSVTAA DTAVYYCARE | sequence |
| | QVPGYYGMDV WGQGTTVTVS | |
| | S | |
| 43 | ggtggctcca tcaacagtaa tagttactac | DNA |
| | | nucleotide |
| | | sequence |
| 44 | GGSINSNSYY | AA amino |
| | | acid |
| | | sequence |
| 45 | atctattatg ataggaacac c | DNA |
| | | nucleotide |
| | | sequence |
| 46 | IYYDRNT | AA amino |
| | | acid |
| | | sequence |
| 47 | gcgagagagc aggtccccgg ctactacggt atggacgtc | DNA |
| | | nucleotide |
| | | sequence |
| 48 | AREQVPGYYG MDV | AA amino |
| | | acid |
| | | sequence |
| 49 | gacatccagt tgacccagtc tccatccttc ctgtctgcat ctgtgggaga cagagtcacc | DNA |
| | atcacttgct gggccagtca gggcattagc agatatttag cctggtatca gcaaaaacca | nucleotide |
| | gggaaaaccc ctaaactcct gatctatgct gcatccactt tgcaaagtgg ggtcccatca | sequence |
| | aggttcagcg gcactggatc tgggacagat ttcactctca caatcagcaa cctgcagcct | |
| | gaagattttg caacttatta ctgtcaacag tttgatagtt acccgtggac gttcggccaa | |
| | gggaccaagg tggaaatcaa a | |
| 50 | DIQLTQSPSF LSASVGDRVT ITCWASQGIS RYLAWYQQKP | AA amino |
| | GKTPKLLIYA ASTLQSGVPS | acid |
| | RFSGTGSGTD FTLTISNLQP EDFATYYCQQ FDSYPWTFGQ | sequence |
| | GTKVEIK | |
| 51 | cagggcatta gcagatat | DNA |
| | | nucleotide |
| | | sequence |
| 52 | QGISRY | AA amino |
| | | acid |
| | | sequence |
| 53 | gctgcatcc | DNA |
| | | nucleotide |
| | | sequence |
| 54 | AAS | AA amino |
| | | acid |
| | | sequence |
| 55 | caacagtttg atagttaccc gtggacg | DNA |
| | | nucleotide |
| | | sequence |
| 56 | QQFDSYPWT | AA amino |
| | | acid |
| | | sequence |
| 57 | cagctgcagc tgcaggagtc gggcccagga ctggtgaagc cttcggagac cctgtccctc | DNA |
| | acctgcactg tctctggtgg ctccatcaac agtaatagtt actactgggg ctggatccgc | nucleotide |
| | cagcccccag ggaagggact ggagtggatt gggagtatct attatgatag gaacacctac | sequence |
| | tacaccccgt ccctcaagag tcgagtcacc atatccgtag acacgtccaa gaaccagttc | |
| | tccctgaagc tgacctctgt gaccgccgca gacacggctg tatattattg tgcgagagag | |
| | caggtccccg gctactacgg tatggacgtc tggggccaag ggaccacggt caccgtctcc | |
| | tcagcctcca ccaagggccc atcggtcttc cccctggcgc cctgctccag gagcacctcc | |
| | gagagcacag ccgccctggg ctgcctggtc aaggactact tccccgaacc ggtgacggtg | |
| | tcgtggaact caggcgccct gaccagcggc gtgcacacct tcccggctgt cctacagtcc | |
| | tcaggactct actccctcag cagcgtggtg accgtgccct ccagcagctt gggcacgaag | |
| | tccaaatatg gtcccccatg cccaccctgc ccagcacctg agttcctggg gggaccatca | |
| | gtcttcctgt tccccccaaa acccaaggac actctcatga tctcccggac ccctgaggtc | |
| | acgtgcgtgg tggtggacgt gagccaggaa gaccccgagg tccagttcaa ctggtacgtg | |
| | gatggcgtgg aggtgcataa tgccaagaca aagccgcggg aggagcagtt caacagcacg | |
| | taccgtgtgg tcagcgtcct caccgtcctg caccaggact ggctgaacgg caaggagtac | |
| | aagtgcaagg tctccaacaa aggcctcccg tcctccatcg agaaaaccat ctccaaagcc | |
| | aaagggcagc cccgagagcc acaggtgtac accctgcccc catcccagga ggagatgacc | |
| | aagaaccagg tcagcctgac ctgcctggtc aaaggcttct accccagcga catcgccgtg | |
| | gagtgggaga gcaatgggca gccggagaac aactacaaga ccacgcctcc cgtgctggac | |
| | tccgacggct ccttcttcct ctacagcagg ctcaccgtgg acaagagcag gtggcaggag | |
| | gggaatgtct tctcatgctc cgtgatgcat gaggctctgc acaaccacta cacacagaag | |
| | tccctctccc tgtctctggg taaatga | |
| 58 | QLQLQESGPG LVKPSETLSL TCTVSGGSIN SNSYYWGWIR | AA amino |
| | QPPGKGLEWI GSIYYDRNTY | acid |
| | YTPSLKSRVT ISVDTSKNQF SLKLTSVTAA DTAVYYCARE | sequence |
| | QVPGYYGMDV WGQGTTVTVS | |
| | SASTKGPSVF PLAPCSRSTS ESTAALGCLV KDYFPEPVTV | |
| | SWNSGALTSG VHTFPAVLQS | |
| | SGLYSLSSVV TVPSSSLGTK TYTCNVDHKP SNTKVDKRVE | |
| | SKYGPPCPPC PAPEFLGGPS | |
| | VFLFPPKPKD TLMISRTPEV TCVVVDVSQE DPEVQFNWYV | |
| | DGVEVHNAKT KPREEQFNST | |
| | YRVVSVLTVL HQDWLNGKEY KCKVSNKGLP SSIEKTISKA | |
| | KGQPREPQVY TLPPSQEEMT | |
| | KNQVSLTCLV KGFYPSDIAV EWESNGQPEN NYKTTPPVLD | |
| | SDGSFFLYSR LTVDKSRWQE | |
| | GNVFSCSVMH EALHNHYTQK SLSLSLGK | |
| 59 | gacatccagt tgacccagtc tccatccttc ctgtctgcat ctgtgggaga cagagtcacc | DNA |
| | atcacttgct gggccagtca gggcattagc agatatttag cctggtatca gcaaaaacca | nucleotide |
| | gggaaaaccc ctaaactcct gatctatgct gcatccactt tgcaaagtgg ggtcccatca | sequence |
| | aggttcagcg gcactggatc tgggacagat ttcactctca caatcagcaa cctgcagcct | |
| | gaagattttg caacttatta ctgtcaacag tttgatagtt acccgtggac gttcggccaa | |
| | gggaccaagg tggaaatcaa acgaactgtg gctgcaccat ctgtcttcat cttcccgcca | |
| | tctgatgagc agttgaaatc tggaactgcc tctgttgtgt gcctgctgaa taacttctat | |
| | cccagagagg ccaaagtaca gtggaaggtg gataacgccc tccaatcggg taactcccag | |
| | gagagtgtca cagagcagga cagcaaggac agcacctaca gcctcagcag caccctgacg | |
| | ctgagcaaag cagactacga gaaacacaaa gtctacgcct gcgaagtcac ccatcagggc | |
| | ctgagctcgc ccgtcacaaa gagcttcaac aggggagagt gttag | |
| 60 | DIQLTQSPSF LSASVGDRVT ITCWASQGIS RYLAWYQQKP | AA amino |
| | GKTPKLLIYA ASTLQSGVPS | acid |
| | RFSGTGSGTD FTLTISNLQP EDFATYYCQQ FDSYPWTFGQ | sequence |
| | GTKVEIKRTV AAPSVFIFPP | |
| | SDEQLKSGTA SVVCLLNNFY PREAKVQWKV DNALQSGNSQ | |
| | ESVTEQDSKD STYSLSSTLT | |
| | LSKADYEKHK VYACEVTHQG LSSPVTKSEN RGEC | |
| 61 | MFVFLVLLPL VSSQCVNLTT RTQLPPAYTN SFTRGVYYPD | AA amino |
| | KVFRSSVLHS TQDLFLPFFS | acid |
| | NVTWFHAIHV SGTNGTKRFD NPVLPFNDGV YFASTEKSNI | sequence |
| | IRGWIFGTTL DSKTQSLLIV | |
| | NNATNVVIKV CEFQFCNDPF LGVYYHKNNK SWMESEFRVY | |
| | SSANNCTFEY VSQPFLMDLE | |
| | GKQGNFKNLR EFVFKNIDGY FKIYSKHTPI NLVRDLPQGF | |
| | SALEPLVDLP IGINITRFQT | |
| | LLALHRSYLT PGDSSSGWTA GAAAYYVGYL QPRTFLLKYN | |
| | ENGTITDAVD CALDPLSETK | |
| | CTLKSFTVEK GIYQTSNFRV QPTESIVRFP NITNLCPFGE | |
| | VFNATRFASV YAWNRKRISN | |
| | CVADYSVLYN SASFSTFKCY GVSPTKLNDL CFTNVYADSF | |
| | VIRGDEVRQI APGQTGKIAD | |
| | YNYKLPDDFT GCVIAWNSNN LDSKVGGNYN YLYRLFRKSN | |
| | LKPFERDIST EIYQAGSTPC | |
| | NGVEGENCYF PLQSYGFQPT NGVGYQPYRV VVLSFELLHA | |
| | PATVCGPKKS TNLVKNKCVN | |
| | FNFNGLTGTG VLTESNKKFL PFQQFGRDIA DTTDAVRDPQ | |
| | TLEILDITPC SFGGVSVITP | |
| | GTNTSNQVAV LYQDVNCTEV PVAIHADQLT PTWRVYSTGS | |
| | NVFQTRAGCL IGAEHVNNSY | |
| | ECDIPIGAGI CASYQTQTNS PRRARSVASQ SIIAYTMSLG | |
| | AENSVAYSNN SIAIPTNFTI | |
| | SVTTEILPVS MTKTSVDCTM YICGDSTECS NLLLQYGSFC | |
| | TQLNRALTGI AVEQDKNTQE | |
| | VFAQVKQIYK TPPIKDFGGF NFSQILPDPS KPSKRSFIED | |
| | LLFNKVTLAD AGFIKQYGDC | |
| | LGDIAARDLI CAQKEFGLTV LPPLLTDEMI AQYTSALLAG | |
| | TITSGWTFGA GAALQIPFAM | |
| | QMAYRFNGIG VTQNVLYENQ KLIANQFNSA IGKIQDSLSS | |
| | TASALGKLQD VVNQNAQALN | |
| | TLVKQLSSNF GAISSVLNDI LSRLDKVEAE VQIDRLITGR | |
| | LQSLQTYVTQ QLIRAAEIRA | |
| | SANLAATKMS ECVLGQSKRV DFCGKGYHLM SFPQSAPHGV | |
| | VFLHVTYVPA QEKNFTTAPA | |
| | ICHDGKAHFP REGVFVSNGT HWFVTQRNFY EPQIITTDNT | |
| | FVSGNCDVVI GIVNNTVYDP | |
| | LQPELDSFKE ELDKYFKNHT SPDVDLGDIS GINASVVNIQ | |
| | KEIDRLNEVA KNLNESLIDL | |
| | QELGKYEQYI KWPWYIWLGF IAGLIAIVMV TIMLCCMTSC | |
| | CSCLKGCCSC GSCCKFDEDD | |
| | SEPVLKGVKL HYT | |
| 62 | GGGGS | AA amino |
| | | acid |
| | | sequence |
| 63 | MALNSGSPPA IGPYYENHGY QPENPYPAQP TVVPTVYEVH | AA amino |
| | PAQYYPSPVP QYAPRVLTQA | acid |
| | SNPVVCTQPK SPSGTVCTSK TKKALCITLT LGTFLVGAAL | sequence |
| | AAGLLWKFMG SKCSNSGIEC | |
| | DSSGTCINPS NWCDGVSHCP GGEDENRCVR LYGPNFILQM | |
| | YSSQRKSWHP VCQDDWNENY | |
| | GRAACRDMGY KNNFYSSQGI VDDSGSTSFM KLNTSAGNVD | |
| | IYKKLYHSDA CSSKAVVSLR | |
| | CIACGVNLNS SRQSRIVGGE SALPGAWPWQ VSLHVQNVHV | |
| | CGGSIITPEW IVTAAHCVEK | |
| | PLNNPWHWTA FAGILRQSFM FYGAGYQVEK VISHPNYDSK | |
| | TKNNDIALMK LQKPLTFNDL | |
| | VKPVCLPNPG MMLQPEQLCW ISGWGATEEK GKTSEVLNAA | |
| | KVLLIETQRC NSRYVYDNLI | |
| | TPAMICAGFL QGNVDSCQGD SGGPLVTSKN NIWWLIGDTS | |
| | WGSGCAKAYR PGVYGNVMVF | |
| | TDWIYRQMRA DG | |
| 64 | MALNSGSPPG VGPYYENHGY QPENPYPAQP TVAPNVYEVH | AA amino |
| | PAQYYPSPVP QYTPRVLTHA | acid |
| | SNPAVCRQPK SPSGTVCTSK TKKALCVTMT LGAVLVGAAL | sequence |
| | AAGLLWKFMG SKCSDSGIEC | |
| | DSSGTCISLS NWCDGVSHCP NGEDENRCVR LYGPNFILQV | |
| | YSSQRKSWHP VCRDDWNENY | |
| | ARAACRDMGY KNSFYSSQGI VDNSGATSFM KLNTSAGNVD | |
| | IYKKLYHSDA CSSKAVVSLR | |
| | CIACGVRSNL SRQSRIVGGQ NALLGAWPWQ VSLHVQNIHV | |
| | CGGSIITPEW IVTAAHCVEK | |
| | PLNSPWQWTA FVGTLRQSSM FYEKGHRVEK VISHPNYDSK | |
| | TKNNDIALMK LHTPLTFNEV | |
| | VKPVCLPNPG MMLEPEQHCW ISGWGATQEK GKTSDVLNAA | |
| | MVPLIEPRRC NNKYVYDGLI | |
| | TPAMICAGFL QGTVDSCQGD SGGPLVTLKN DVWWLIGDTS | |
| | WGSGCAQANR PGVYGNVTVF | |
| | TDWIYRQMRA DG | |

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent identified even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1.-40. (canceled)

41. An isolated recombinant antibody or antigen-binding fragment thereof that specifically binds to human transmembrane protease, serine 2 (TMPRSS2), wherein the antibody comprises:
(a) a heavy chain complementarity determining region (HCDR)1 amino acid sequence of SEQ ID NO: 4 with no more than one amino acid substitution; an HCDR2 amino acid sequence of SEQ ID NO: 6 with no more than one amino acid substitution; an HCDR3 amino acid sequence of SEQ ID NO: 8 with no more than one amino acid substitution; an LCDR1 amino acid sequence of SEQ ID NO: 12 with no more than one amino acid substitution; an LCDR2 amino acid sequence of SEQ ID NO: 14 with no more than one amino acid substitution; and an LCDR3 amino acid sequence of SEQ ID NO: 16 with no more than one amino acid substitution;
(b) an HCDR1 amino acid sequence of SEQ ID NO: 24 with no more than one amino acid substitution; an HCDR2 amino acid sequence of SEQ ID NO: 26 with no more than one amino acid substitution; an HCDR3 amino acid sequence of SEQ ID NO: 28 with no more than one amino acid substitution; an LCDR1 amino acid sequence of SEQ ID NO: 32 with no more than one amino acid substitution; an LCDR2 amino acid sequence of SEQ ID NO: 34 with no more than one amino acid substitution; and an LCDR3 amino acid sequence of SEQ ID NO: 36 with no more than one amino acid substitution; or
(c) an HCDR1 amino acid sequence of SEQ ID NO: 44 with no more than one amino acid substitution; an HCDR2 amino acid sequence of SEQ ID NO: 46 with no more than one amino acid substitution; an HCDR3 amino acid sequence of SEQ ID NO: 48 with no more than one amino acid substitution; an LCDR1 amino acid sequence of SEQ ID NO: 52 with no more than one amino acid substitution; an LCDR2 amino acid sequence of SEQ ID NO: 54 with no more than one amino acid substitution; and an LCDR3 amino acid sequence of SEQ ID NO: 56 with no more than one amino acid substitution.

42. The isolated recombinant antibody or antigen-binding fragment thereof of claim 41 comprising:
(a) a heavy chain variable region (HCVR) amino acid sequence which contains the HCDR1, HCDR2 and HCDR3 having at least 90% sequence identity to an amino acid sequence of SEQ ID NO: 2 and a light chain variable region (LCVR) amino acid sequence which contains the LCDR1, LCDR2 and LCDR3 having at least 90% sequence identity to an amino acid sequence of SEQ ID NO: 10;
(b) an HCVR amino acid sequence which contains the HCDR1, HCDR2 and HCDR3 having at least 90% sequence identity to an amino acid sequence of SEQ ID NO: 22 and an LCVR amino acid sequence which contains the LCDR1, LCDR2 and LCDR3 having at least 90% sequence identity to an amino acid sequence of SEQ ID NO: 30; or
(c) an HCVR amino acid sequence which contains the HCDR1, HCDR2 and HCDR3 having at least 90% sequence identity to an amino acid sequence of SEQ ID NO: 42 and an LCVR amino acid sequence which contains the LCDR1, LCDR2 and LCDR3 having at least 90% sequence identity to an amino acid sequence of SEQ ID NO: 50.

43. The isolated recombinant antibody or antigen-binding fragment thereof of claim 41 comprising:
(a) a heavy chain variable region (HCVR) amino acid sequence which contains the HCDR1, HCDR2 and HCDR3 having at least 95% sequence identity to an amino acid sequence of SEQ ID NO: 2 and a light chain variable region (LCVR) amino acid sequence which contains the LCDR1, LCDR2 and LCDR3 having at least 95% sequence identity to an amino acid sequence of SEQ ID NO: 10;
(b) an HCVR amino acid sequence which contains the HCDR1, HCDR2 and HCDR3 having at least 95% sequence identity to an amino acid sequence of SEQ ID NO: 22 and an LCVR amino acid sequence which contains the LCDR1, LCDR2 and LCDR3 having at least 95% sequence identity to an amino acid sequence of SEQ ID NO: 30; or
(c) an HCVR amino acid sequence which contains the HCDR1, HCDR2 and HCDR3 having at least 95% sequence identity to an amino acid sequence of SEQ ID NO: 42 and an LCVR amino acid sequence which contains the LCDR1, LCDR2 and LCDR3 having at least 95% sequence identity to an amino acid sequence of SEQ ID NO: 50.

44. The isolated recombinant antibody or antigen-binding fragment thereof of claim 41 comprising:
(a) a heavy chain variable region (HCVR) amino acid sequence which contains the HCDR1, HCDR2 and HCDR3 having at least 98% sequence identity to an amino acid sequence of SEQ ID NO: 2 and a light chain variable region (LCVR) amino acid sequence which contains the LCDR1, LCDR2 and LCDR3 having at least 98% sequence identity to an amino acid sequence of SEQ ID NO: 10;
(b) an HCVR amino acid sequence which contains the HCDR1, HCDR2 and HCDR3 having at least 98% sequence identity to an amino acid sequence of SEQ ID NO: 22 and an LCVR amino acid sequence which contains the LCDR1, LCDR2 and LCDR3 having at least 98% sequence identity to an amino acid sequence of SEQ ID NO: 30; or
(c) an HCVR amino acid sequence which contains the HCDR1, HCDR2 and HCDR3 having at least 98% sequence identity to an amino acid sequence of SEQ ID NO: 42 and an LCVR amino acid sequence which contains the LCDR1, LCDR2 and LCDR3 having at least 98% sequence identity to an amino acid sequence of SEQ ID NO: 50.

45. The isolated recombinant antibody or antigen-binding fragment thereof of claim 41 comprising:
(a) a heavy chain variable region (HCVR) amino acid sequence which contains the HCDR1, HCDR2 and HCDR3 having at least 99% sequence identity to an amino acid sequence of SEQ ID NO: 2 and a light chain variable region (LCVR) amino acid sequence which contains the LCDR1, LCDR2 and LCDR3 having at least 99% sequence identity to an amino acid sequence of SEQ ID NO: 10;
(b) an HCVR amino acid sequence which contains the HCDR1, HCDR2 and HCDR3 having at least 99% sequence identity to an amino acid sequence of SEQ ID NO: 22 and an LCVR amino acid sequence which contains the LCDR1, LCDR2 and LCDR3 having at least 99% sequence identity to an amino acid sequence of SEQ ID NO: 30; or
(c) an HCVR amino acid sequence which contains the HCDR1, HCDR2 and HCDR3 having at least 99% sequence identity to an amino acid sequence of SEQ ID NO: 42 and an LCVR amino acid sequence which contains the LCDR1, LCDR2 and LCDR3 having at least 99% sequence identity to an amino acid sequence of SEQ ID NO: 50.

46. The isolated recombinant antibody or antigen-binding fragment thereof of claim 41 comprising:
(a) an HCVR amino acid sequence of SEQ ID NO: 2 with no more than 3 amino acid substitutions and an LCVR amino acid sequence of SEQ ID NO: 10 with no more than 3 amino acid substitutions;
(b) an HCVR amino acid sequence of SEQ ID NO: 22 with no more than 3 amino acid substitutions and an LCVR amino acid sequence of SEQ ID NO: 30 with no more than 3 amino acid substitutions; or
(c) an HCVR amino acid sequence of SEQ ID NO: 42 with no more than 3 amino acid substitutions and an LCVR amino acid sequence of SEQ ID NO: 50 with no more than 3 amino acid substitutions.

47. The isolated recombinant antibody or antigen-binding fragment thereof of claim 41 comprising:
(a) an HCVR amino acid sequence of SEQ ID NO: 2 with no more than 2 amino acid substitutions and an LCVR amino acid sequence of SEQ ID NO: 10 with no more than 2 amino acid substitutions;
(b) an HCVR amino acid sequence of SEQ ID NO: 22 with no more than 2 amino acid substitutions and an LCVR amino acid sequence of SEQ ID NO: 30 with no more than 2 amino acid substitutions; or
(c) an HCVR amino acid sequence of SEQ ID NO: 42 with no more than 2 amino acid substitutions and an LCVR amino acid sequence of SEQ ID NO: 50 with no more than 2 amino acid substitutions.

48. The isolated recombinant antibody or antigen-binding fragment thereof of claim 41 comprising:
(a) an HCVR amino acid sequence of SEQ ID NO: 2 with no more than 1 amino acid substitutions and an LCVR amino acid sequence of SEQ ID NO: 10 with no more than 1 amino acid substitutions;
(b) an HCVR amino acid sequence of SEQ ID NO: 22 with no more than 1 amino acid substitutions and an LCVR amino acid sequence of SEQ ID NO: 30 with no more than 1 amino acid substitutions; or
(c) an HCVR amino acid sequence of SEQ ID NO: 42 with no more than 1 amino acid substitutions and an LCVR amino acid sequence of SEQ ID NO: 50 with no more than 1 amino acid substitutions.

49. The isolated recombinant antibody or antigen-binding fragment thereof of claim 41 comprising:
(a) a heavy chain amino acid sequence which contains the HCDR1, HCDR2 and HCDR3 having at least 85% sequence identity to an amino acid sequence of SEQ ID NO: 18 and a light chain amino acid sequence which contains the LCDR1, LCDR2 and LCDR3 having at least 85% sequence identity to an amino acid sequence of SEQ ID NO: 20;
(b) a heavy chain amino acid sequence which contains the HCDR1, HCDR2 and HCDR3 having at least 85% sequence identity to an amino acid sequence of SEQ ID NO: 38 and a light chain amino acid sequence which contains the LCDR1, LCDR2 and LCDR3 having at least 85% sequence identity to an amino acid sequence of SEQ ID NO: 40; or
(c) a heavy chain amino acid sequence which contains the HCDR1, HCDR2 and HCDR3 having at least 85% sequence identity to an amino acid sequence of SEQ ID NO: 58 and a light chain amino acid sequence which contains the LCDR1, LCDR2 and LCDR3 having at least 85% sequence identity to an amino acid sequence of SEQ ID NO: 60.

50. The isolated recombinant antibody or antigen-binding fragment thereof of claim 41 comprising:
(a) a heavy chain amino acid sequence which contains the HCDR1, HCDR2 and HCDR3 having at least 90% sequence identity to an amino acid sequence of SEQ ID NO: 18 and a light chain amino acid sequence which contains the LCDR1, LCDR2 and LCDR3 having at least 90% sequence identity to an amino acid sequence of SEQ ID NO: 20;
(b) a heavy chain amino acid sequence which contains the HCDR1, HCDR2 and HCDR3 having at least 90% sequence identity to an amino acid sequence of SEQ ID NO: 38 and a light chain amino acid sequence which contains the LCDR1, LCDR2 and LCDR3 having at least 90% sequence identity to an amino acid sequence of SEQ ID NO: 40; or
(c) a heavy chain amino acid sequence which contains the HCDR1, HCDR2 and HCDR3 having at least 90% sequence identity to an amino acid sequence of SEQ ID NO: 58 and a light chain amino acid sequence which contains the LCDR1, LCDR2 and LCDR3 having at least 90% sequence identity to an amino acid sequence of SEQ ID NO: 60.

51. The isolated recombinant antibody or antigen-binding fragment thereof of claim 41 comprising:
(a) a heavy chain amino acid sequence which contains the HCDR1, HCDR2 and HCDR3 having at least 95% sequence identity to an amino acid sequence of SEQ ID NO: 18 and a light chain amino acid sequence which contains the LCDR1, LCDR2 and LCDR3 having at least 95% sequence identity to an amino acid sequence of SEQ ID NO: 20;
(b) a heavy chain amino acid sequence which contains the HCDR1, HCDR2 and HCDR3 having at least 95% sequence identity to an amino acid sequence of SEQ ID NO: 38 and a light chain amino acid sequence which contains the LCDR1, LCDR2 and LCDR3 having at least 95% sequence identity to an amino acid sequence of SEQ ID NO: 40; or
(c) a heavy chain amino acid sequence which contains the HCDR1, HCDR2 and HCDR3 having at least 95% sequence identity to an amino acid sequence of SEQ ID NO: 58 and a light chain amino acid sequence which contains the LCDR1, LCDR2 and LCDR3 having at least 95% sequence identity to an amino acid sequence of SEQ ID NO: 60.

52. The isolated recombinant antibody or antigen-binding fragment thereof of claim 41 comprising:
(a) a heavy chain amino acid sequence which contains the HCDR1, HCDR2 and HCDR3 having at least 98% sequence identity to an amino acid sequence of SEQ ID NO: 18 and a light chain amino acid sequence which contains the LCDR1, LCDR2 and LCDR3 having at least 98% sequence identity to an amino acid sequence of SEQ ID NO: 20;
(b) a heavy chain amino acid sequence which contains the HCDR1, HCDR2 and HCDR3 having at least 98% sequence identity to an amino acid sequence of SEQ ID NO: 38 and a light chain amino acid sequence which contains the LCDR1, LCDR2 and LCDR3 having at least 98% sequence identity to an amino acid sequence of SEQ ID NO: 40; or
(c) a heavy chain amino acid sequence which contains the HCDR1, HCDR2 and HCDR3 having at least 98% sequence identity to an amino acid sequence of SEQ ID NO: 58 and a light chain amino acid sequence which contains the LCDR1, LCDR2 and LCDR3 having at least 98% sequence identity to an amino acid sequence of SEQ ID NO: 60.

53. The isolated recombinant antibody or antigen-binding fragment thereof of claim 41 comprising:
(a) a heavy chain amino acid sequence which contains the HCDR1, HCDR2 and HCDR3 having at least 99% sequence identity to an amino acid sequence of SEQ ID NO: 18 and a light chain amino acid sequence which contains the LCDR1, LCDR2 and LCDR3 having at least 99% sequence identity to an amino acid sequence of SEQ ID NO: 20;
(b) a heavy chain amino acid sequence which contains the HCDR1, HCDR2 and HCDR3 having at least 99% sequence identity to an amino acid sequence of SEQ ID NO: 38 and a light chain amino acid sequence which contains the LCDR1, LCDR2 and LCDR3 having at least 99% sequence identity to an amino acid sequence of SEQ ID NO: 40; or
(c) a heavy chain amino acid sequence which contains the HCDR1, HCDR2 and HCDR3 having at least 99% sequence identity to an amino acid sequence of SEQ ID NO: 58 and a light chain amino acid sequence which contains the LCDR1, LCDR2 and LCDR3 having at least 99% sequence identity to an amino acid sequence of SEQ ID NO: 60.

54. The antibody or antigen-binding fragment thereof of claim 41 which is multispecific.

55. The antibody or antigen-binding fragment thereof of claim 41 which comprises one or more of the following properties:
a) inhibits growth of coronavirus in TMPRSS2-expressing cells;
b) inhibits growth of influenza virus in TMPRSS2-expressing cells;
c) binds to the surface of TMPRSS-expressing cells;
d) does not significantly bind to MDCK/Tet-on cells which do not express TMPRSS2;
e) limits spread of coronavirus infection of cells in vitro;
f) limits spread of influenza virus infection of cells in vitro;
g) protects mice engineered to express the human TMPRSS2 protein from death and/or weight loss caused by coronavirus infection; and
h) protects mice engineered to express the human TMPRSS2 protein from death caused by influenza virus infection.

56. A complex comprising the antibody or antigen-binding fragment thereof of claim 41 bound to a TMPRSS2 polypeptide.

57. A method for making the antibody or antigen-binding fragment thereof of claim 41, comprising:
(a) introducing into a host cell one or more polynucleotides encoding said antibody or antigen-binding fragment thereof;
(b) culturing the host cell under conditions favorable to expression of the one or more polynucleotides; and
(c) optionally, isolating the antibody or antigen-binding fragment thereof from the host cell and/or medium in which the host cell is grown.

58. The method of claim 57, wherein the host cell is a Chinese hamster ovary cell.

59. An antibody or antigen-binding fragment thereof which is a product of the method of claim 57.

60. A composition comprising:
(i) a first polynucleotide encoding a polypeptide comprising (a) a CDR-H1 comprising SEQ ID NO: 4 with no more than one amino acid substitution, (b) a CDR-H2 comprising SEQ ID NO: 6 with no more than one amino acid substitution, and (c) a CDR-H3 comprising SEQ ID NO: 8 with no more than one amino acid substitution; and a second polynucleotide encoding a polypeptide comprising (a) a CDR-L1 comprising SEQ ID NO: 12 with no more than one amino acid substitution, (b) a CDR-L2 comprising SEQ ID NO: 14 with no more than one amino acid substitution, and (c) a CDR-L3 comprising SEQ ID NO: 16 with no more than one amino acid substitution;
(ii) a first polynucleotide encoding a polypeptide comprising (a) a CDR-H1 comprising SEQ ID NO: 24 with no more than one amino acid substitution, (b) a CDR-H2 comprising SEQ ID NO: 26 with no more than one amino acid substitution, and (c) a CDR-H3 comprising SEQ ID NO: 28; with no more than one amino acid substitution and a second polynucleotide encoding a polypeptide comprising (a) CDR-L1 comprising SEQ ID NO: 32 with no more than one amino acid substitution, (b) a CDR-L2 comprising SEQ ID NO: 34 with no more than one amino acid substitution, and (c) a CDR-L3 comprising SEQ ID NO: 36 with no more than one amino acid substitution; or
(iii) a first polynucleotide encoding a polypeptide comprising (a) a CDR-H1 comprising SEQ ID NO: 44 with no more than one amino acid substitution, (b) a CDR-H2 comprising SEQ ID NO: 46 with no more than one amino acid substitution, and (c) a CDR-H3 comprising SEQ ID NO: 48 with no more than one amino acid substitution; and a second polynucleotide encoding a polypeptide comprising (a) a CDR-L1 comprising SEQ ID NO: 52 with no more than one amino acid substitution, (b) a CDR-L2 comprising SEQ ID NO: 54 with no more than one amino acid substitution, and (c) a CDR-L3 comprising SEQ ID NO: 56 with no more than one amino acid substitution.

61. A vector comprising:
(i) a first polynucleotide encoding a polypeptide comprising (a) a CDR-H1 comprising SEQ ID NO: 4 with no more than one amino acid substitution, (b) a CDR-H2 comprising SEQ ID NO: 6 with no more than one amino acid substitution, and (c) a CDR-H3 comprising SEQ ID NO:8 with no more than one amino acid substitution; and a second polynucleotide encoding a polypeptide comprising (a) a CDR-L1 comprising SEQ ID NO: 12 with no more than one amino acid substitution, (b) a CDR-L2 comprising SEQ ID NO: 14 with no more than one amino acid substitution, and (c) a CDR-L3 comprising SEQ ID NO: 16 with no more than one amino acid substitution;
(ii) a first polynucleotide encoding a polypeptide comprising (a) a CDR-H1 comprising SEQ ID NO: 4 with no more than one amino acid substitution, (b) a CDR-H2 comprising SEQ ID NO: 6 with no more than one amino acid substitution, and (c) a CDR-H3 comprising SEQ ID NO: 8 with no more than one amino acid substitution; and a second polynucleotide encoding a polypeptide comprising (a) a CDR-L1 comprising SEQ ID NO: 12 with no more than one amino acid substitution, (b) a CDR-L2 comprising SEQ ID NO: 14 with no more than one amino acid substitution, and (c) a CDR-L3 comprising SEQ ID NO: 16 with no more than one amino acid substitution; or
(iii) a first polynucleotide encoding a polypeptide comprising (a) a CDR-H1 comprising SEQ ID NO: 44 with no more than one amino acid substitution, (b) a CDR-H2 comprising SEQ ID NO: 46 with no more than one amino acid substitution, and (c) a CDR-H3 comprising SEQ ID NO: 48 with no more than one amino acid substitution; and a second polynucleotide encoding a polypeptide comprising (a) a CDR-L1 comprising SEQ ID NO: 52 with no more than one amino acid substitution, (b) a CDR-L2 comprising SEQ ID NO: 54 with no more than one amino acid substitution, and (c) a CDR-L3 comprising SEQ ID NO: 56 with no more than one amino acid substitution.

62. A host cell comprising the vector of claim 61.

63. A composition or kit comprising the antibody or antigen-binding fragment thereof of claim 41.

64. The composition or kit of claim 63 comprising a further therapeutic agent selected from the group consisting of an anti-viral drug, a vaccine, an antibody or antigen-binding fragment thereof that binds to influenza Group I HA protein, and an antibody or antigen-binding fragment thereof that binds to influenza Group II HA protein.

65. The composition or kit of claim 64 wherein the further therapeutic agent is a member selected from the group consisting of remdesivir, chloroquine, lopinavir, ritonavir, ribavirin, ledipasvir, sofosbuvir, a combination of ledipasvir and sofosbuvir, oseltamivir, zanamivir, ribavirin and interferon-alpha2b, interferon-alpha2a, an anti-cancer agent, and an antibody or antigen-binding fragment thereof that specifically binds to influenza HA or coronavirus spike protein; and/or
an antibody or antigen binding fragment thereof selected from the group consisting of H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; H1H18335B; H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2.

66. A pharmaceutical composition comprising the antibody or antigen-binding fragment thereof of claim 41 and a pharmaceutically acceptable carrier.

67. The pharmaceutical composition of claim 66, further comprising a therapeutic agent selected from the group consisting of an anti-viral drug, a vaccine, an antibody or antigen-binding fragment thereof that binds to influenza Group I HA protein, and an antibody or antigen-binding fragment thereof that binds to influenza Group II HA protein.

68. A vessel or injection device comprising the antibody or antigen-binding fragment thereof of claim 41.

69. A method for treating prostate, renal, or pancreatic cancer or infection with an influenza virus, coronavirus, SARS-CoV, MERS-CoV, SARS-CoV-2, or parainfluenza virus in a subject in need thereof, comprising administering a therapeutically effective amount of the antibody or antigen-binding fragment thereof of claim 41.

70. The method of claim 69, wherein the subject is administered one or more further therapeutic agents selected from the group consisting of an anti-viral drug, a vaccine, an antibody or antigen-binding fragment thereof that binds to influenza Group I HA protein, and an antibody or antigen-binding fragment thereof that binds to influenza Group II HA protein.

71. The method of claim 70, wherein the subject is administered one or more further therapeutic agents which is an anti-viral drug or a vaccine.

72. The method of claim 70, wherein the subject is administered one or more further therapeutic agents which is a member selected from the group consisting of: remdesivir, chloroquine, lopinavir, ritonavir, ribavirin, ledipasvir, sofosbuvir, a combination of ledipasvir and sofosbuvir, oseltamivir, zanamivir, ribavirin and interferon-alpha2b, interferon-alpha2a, and an antibody or antigen-binding fragment thereof that specifically binds to influenza HA or coronavirus spike protein; and/or an antibody or antigen binding fragment thereof selected from the group consisting of
H1H11723P; H1H11729P; H1H11820N; H1H11829N; H1H11829N2; H2aM11829N; H2M11830N; H1H11830N2; H1H11903N; H1H14571N; H2a14571N; H1H11704P; H1H11711P; H1H11714P; H1H11717P; H1H11724P; H1H11727P; H1H11730P2; H1H11731P2; H1H11734P2; H1H11736P2; H1H11742P2; H1H11744P2; H1H11745P2; H1H11747P2; H1H11748P2; H1H17952B; H1H17953B; H1H17954B; H1H17955B; H1H17956B; H1H17957B; H1H17958B; H1H17959B; H1H17960B; H1H17961B; H1H17962B; H1H17963B; H1H17964B; H1H17965B; H1H17966B; H1H17967B; H1H17968B; H1H17969B; H1H17970B; H1H17971B; H1H17972B; H1H17973B; H1H17974B; H1H17975B; H1H17976B; H1H17977B; H1H17978B; H1H17979B; H1H17980B; H1H17981B; H1H17982B; H1H17983B; H1H17984B; H1H17985B; H1H17986B; H1H17987B; H1H17988B; H1H17989B; H1H17990B; H1H17991B; H1H17992B; H1H17993B; H1H17994B; H1H17995B; H1H17996B; H1H17997B; H1H17998B; H1H17999B; H1H18000B; H1H18001B; H1H18002B; H1H18003B; H1H18004B; H1H18005B; H1H18006B; H1H18007B; H1H18008B; H1H18009B; H1H18010B; H1H18011B; H1H18012B; H1H18013B; H1H18014B; H1H18015B; H1H18016B; H1H18017B; H1H18018B; H1H18019B; H1H18020B; H1H18021B; H1H18022B; H1H18023B; H1H18024B; H1H18025B; H1H18026B; H1H18027B; H1H18028B; H1H18029B; H1H18030B; H1H18031B; H1H18032B; H1H18033B; H1H18034B; H1H18035B; H1H18037B; H1H18038B; H1H18039B; H1H18040B; H1H18041B; H1H18042B; H1H18043B; H1H18044B; H1H18045B; H1H18046B; H1H18047B; H1H18048B; H1H18049B; H1H18051B; H1H18052B; H1H18053B; H1H18054B; H1H18055B; H1H18056B; H1H18057B; H1H18058B; H1H18059B; H1H18060B; H1H18061B; H1H18062B; H1H18063B; H1H18064B; H1H18065B; H1H18066B; H1H18067B; H1H18068B; H1H18069B; H1H18070B; H1H18071B; H1H18072B; H1H18073B; H1H18074B; H1H18075B; H1H18076B; H1H18077B; H1H18078B; H1H18079B; H1H18080B; H1H18081B; H1H18082B; H1H18083B; H1H18084B; H1H18085B; H1H18086B; H1H18087B; H1H18088B; H1H18089B; H1H18090B; H1H18091B; H1H18092B; H1H18093B; H1H18094B; H1H18095B; H1H18096B; H1H18097B; H1H18098B; H1H18099B; H1H18100B; H1H18101B; H1H18102B; H1H18103B; H1H18104B; H1H18105B; H1H18107B; H1H18108B; H1H18109B; H1H18110B; H1H18111B; H1H18112B; H1H18113B; H1H18114B; H1H18115B; H1H18116B; H1H18117B; H1H18118B; H1H18119B; H1H18120B; H1H18121B; H1H18122B; H1H18123B; H1H18124B; H1H18125B; H1H18126B; H1H18127B; H1H18128B; H1H18129B; H1H18130B; H1H18131B; H1H18132B; H1H18133B; H1H18134B; H1H18135B; H1H18136B; H1H18137B; H1H18138B; H1H18139B; H1H18140B; H1H18141B; H1H18142B; H1H18143B; H1H18144B; H1H18145B; H1H18146B; H1H18147B; H1H18148B; H1H18149B; H1H18150B; H1H18151B; H1H18152B; H1H18153B; H1H18154B; H1H18155B; H1H18156B; H1H18157B; H1H18158B; H1H18159B; H1H18160B; H1H18161B; H1H18162B; H1H18163B; H1H18164B; H1H18165B; H1H18166B; H1H18167B; H1H18168B; H1H18169B; H1H18170B; H1H18171B; H1H18172B; H1H18173B; H1H18174B; H1H18175B; H1H18176B; H1H18177B; H1H18178B; H1H18179B; H1H18180B; H1H18181B; H1H18182B; H1H18183B; H1H18184B; H1H18185B; H1H18186B; H1H18187B; H1H18188B; H1H18189B; H1H18190B; H1H18191B; H1H18192B; H1H18193B; H1H18194B; H1H18195B; H1H18196B; H1H18197B; H1H18198B; H1H18199B; H1H18200B; H1H18201B; H1H18202B; H1H18203B; H1H18204B; H1H18205B; H1H18206B; H1H18207B; H1H18208B; H1H18209B; H1H18210B; H1H18211B; H1H18212B; H1H18213B; H1H18214B; H1H18216B; H1H18217B; H1H18218B; H1H18219B; H1H18220B; H1H18221B; H1H18222B; H1H18223B; H1H18224B; H1H18225B; H1H18226B; H1H18227B; H1H18228B; H1H18229B; H1H18230B; H1H18231B; H1H18232B; H1H18233B; H1H18234B; H1H18235B; H1H18236B; H1H18237B; H1H18238B; H1H18239B; H1H18240B; H1H18241B; H1H18242B; H1H18243B; H1H18244B; H1H18245B; H1H18246B; H1H18247B; H1H18248B; H1H18249B; H1H18250B; H1H18251B; H1H18252B; H1H18253B; H1H18254B; H1H18255B; H1H18256B; H1H18257B; H1H18258B; H1H18259B; H1H18261B; H1H18262B; H1H18263B; H1H18264B; H1H18265B; H1H18266B; H1H18267B; H1H18268B; H1H18269B; H1H18270B; H1H18271B; H1H18272B; H1H18274B; H1H18275B; H1H18276B; H1H18277B; H1H18278B; H1H18279B; H1H18280B; H1H18281B; H1H18282B; H1H18283B; H1H18284B; H1H18285B; H1H18286B; H1H18287B; H1H18288B; H1H18289B; H1H18290B; H1H18291B; H1H18292B; H1H18293B; H1H18294B; H1H18295B; H1H18297B; H1H18298B; H1H18299B; H1H18300B; H1H18301B; H1H18302B; H1H18303B; H1H18304B; H1H18305B; H1H18306B; H1H18307B; H1H18308B; H1H18309B; H1H18310B; H1H18311B; H1H18312B; H1H18313B; H1H18314B; H1H18315B; H1H18316B; H1H18317B; H1H18318B; H1H18319B; H1H18320B; H1H18321B; H1H18322B; H1H18323B; H1H18324B; H1H18325B; H1H18326B; H1H18327B; H1H18328B; H1H18329B; H1H18330B; H1H18331B; H1H18332B; H1H18333B; H1H18334B; H1H18335B; H4sH15188P; H1H15188P; H1H15211P; H1H15177P; H4sH15211P; H1H15260P2; H1H15259P2; H1H15203P; H4sH15260P2; H4sH15231P2; H1H15237P2; H1H15208P; H1H15228P2; H1H15233P2; H1H15264P2; H1H15231P2; H1H15253P2; H1H15215P; and H1H15249P2.

73. A method for administering the antibody or antigen-binding fragment thereof of claim 41 into the body of a subject comprising injecting the antibody or antigen-binding fragment thereof into the body of the subject.

74. The method of claim 73, wherein the antibody or antigen-binding fragment thereof is injected into the body of the subject subcutaneously, intravenously or intramuscularly.

75. The antibody or antigen-binding fragment thereof of claim 41, which binds to TMPRSS2 with an EC₅₀ of less than about 10⁻⁹ M.

76. The antibody or antigen-binding fragment thereof of claim 41, which demonstrates an increase in survival in a coronavirus-infected animal after administration to said coronavirus-infected animal, as compared to a comparable coronavirus-infected animal without said administration.


SEQUENCE LISTING (64 sequences)

SEQ ID NO: 1 - DNA, 357 bp, synthetic construct
  misc_feature 1..357: note = Synthetic
  source 1..357: mol_type = other DNA
gaggtgcagctggtggagtctgggggaggcttggtacagcctggggggtccctgagactctcctgtgaagcctctggattcaccttcaggagctacgacatgcactgggtccgccaagctacaggaaaaggtctggagtgggtctcagctattggttctgctggtgacacactctatccagactccgtgaagggccgattcaccatctccagagagaatgccaagaactccttgtatcttcaaatggacagcctgagagccggggacacggctgtgtattactgtgcaagagttggagactggggatcggggtacttagactactggggccagggaaccctggtcaccgtctcctca

SEQ ID NO: 2 - protein, 119 aa, synthetic construct
  REGION 1..119: note = Synthetic
EVQLVESGGGLVQPGGSLRLSCEASGFTFRSYDMHWVRQATGKGLEWVSAIGSAGDTLYPDSVKGRFTISRENAKNSLYLQMDSLRAGDTAVYYCARVGDWGSGYLDYWGQGTLVTVSS

SEQ ID NO: 3 - DNA, 24 bp, synthetic construct
  misc_feature 1..24: note = Synthetic
  source 1..24: mol_type = other DNA
ggattcaccttcaggagctacgac

SEQ ID NO: 4 - protein, 8 aa, synthetic construct
  REGION 1..8: note = Synthetic
GFTFRSYD

SEQ ID NO: 5 - DNA, 21 bp, synthetic construct
  misc_feature 1..21: note = Synthetic
  source 1..21: mol_type = other DNA
attggttctgctggtgacaca

SEQ ID NO: 6 - protein, 7 aa, synthetic construct
  REGION 1..7: note = Synthetic
IGSAGDT

SEQ ID NO: 7 - DNA, 39 bp, synthetic construct
  misc_feature 1..39: note = Synthetic
  source 1..39: mol_type = other DNA
gcaagagttggagactggggatcggggtacttagactac

SEQ ID NO: 8 - protein, 13 aa, synthetic construct
  REGION 1..13: note = Synthetic
ARVGDWGSGYLDY

SEQ ID NO: 9 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagcattagcatctatttaaattggtatcagcagaaaccagggagagcccctaaactcctgatctatgttgcatccagtttgcaaagtggggtcccatcaaggttcagtggcagtggatctgggacagatttcactctcaccatcagcagtctgcaacctgaagattttgcaacttactactgtcaacagagttacggtaccccattcactttcggccctgggaccaaagtggatatcaaa

SEQ ID NO: 10 - protein, 107 aa, synthetic construct
  REGION 1..107: note = Synthetic
DIQMTQSPSSLSASVGDRVTITCRASQSISIYLNWYQQKPGRAPKLLIYVASSLQSGVPSRFSGSGSGTDFTLTISSLQPEDFATYYCQQSYGTPFTFGPGTKVDIK

SEQ ID NO: 11 - DNA, 18 bp, synthetic construct
  misc_feature 1..18: note = Synthetic
  source 1..18: mol_type = other DNA
cagagcattagcatctat

SEQ ID NO: 12 - protein, 6 aa, synthetic construct
  REGION 1..6: note = Synthetic
QSISIY

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
caacagagttacggtaccccattcact

SEQ ID NO: 16 - protein, 9 aa, synthetic construct
  REGION 1..9: note = Synthetic
QQSYGTPFT

SEQ ID NO: 17 - DNA, 1341 bp, synthetic construct
  misc_feature 1..1341: note = Synthetic
  source 1..1341: mol_type = other DNA
gaggtgcagctggtggagtctgggggaggcttggtacagcctggggggtccctgagactctcctgtgaagcctctggattcaccttcaggagctacgacatgcactgggtccgccaagctacaggaaaaggtctggagtgggtctcagctattggttctgctggtgacacactctatccagactccgtgaagggccgattcaccatctccagagagaatgccaagaactccttgtatcttcaaatggacagcctgagagccggggacacggctgtgtattactgtgcaagagttggagactggggatcggggtacttagactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 446 aa, synthetic construct
  REGION 1..446: note = Synthetic
EVQLVESGGGLVQPGGSLRLSCEASGFTFRSYDMHWVRQATGKGLEWVSAIGSAGDTLYPDSVKGRFTISRENAKNSLYLQMDSLRAGDTAVYYCARVGDWGSGYLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 645 bp, synthetic construct
  misc_feature 1..645: note = Synthetic
  source 1..645: mol_type = other DNA
gacatccagatgacccagtctccatcctccctgtctgcatctgtaggagacagagtcaccatcacttgccgggcaagtcagagcattagcatctatttaaattggtatcagcagaaaccagggagagcccctaaactcctgatctatgttgcatccagtttgcaaagtggggtcccatcaaggttcagtggcagtggatctgggacagatttcactctcaccatcagcagtctgcaacctgaagattttgcaacttactactgtcaacagagttacggtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, synthetic construct
  REGION 1..214: note = Synthetic
DIQMTQSPSSLSASVGDRVTITCRASQSISIYLNWYQQKPGRAPKLLIYVASSLQSGVPSRFSGSGSGTDFTLTISSLQPEDFATYYCQQSYGTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 351 bp, synthetic construct
  misc_feature 1..351: note = Synthetic
  source 1..351: mol_type = other DNA
caggttcagctggtgcagtctggagctgaggtgaagaagcctggggcctcagtgagggtctcctgcaaggcttctggttacacctttaccaattatggtatcacctgggtgcgacaggcccctggacaagggcttgagtggatgggagggatcagcgtttacaatggtaacacagaatatgcagagaaattccagggcagagtctccatgaccacagacacatccacgaccacagcctacttggagctgaggagcctgaaatctgacgacacggccgtctatttctgtgcgagagggcaactggactacttcgacccctggggccagggaaccctggtcactgtctcctca

SEQ ID NO: 22 - protein, 117 aa, synthetic construct
  REGION 1..117: note = Synthetic
QVQLVQSGAEVKKPGASVRVSCKASGYTFTNYGITWVRQAPGQGLEWMGGISVYNGNTEYAEKFQGRVSMTTDTSTTTAYLELRSLKSDDTAVYFCARGQLDYFDPWGQGTLVTVSS

SEQ ID NO: 23 - DNA, 24 bp, synthetic construct
  misc_feature 1..24: note = Synthetic
  source 1..24: mol_type = other DNA
ggttacacctttaccaattatggt

SEQ ID NO: 24 - protein, 8 aa, synthetic construct
  REGION 1..8: note = Synthetic
GYTFTNYG

SEQ ID NO: 25 - DNA, 24 bp, synthetic construct
  misc_feature 1..24: note = Synthetic
  source 1..24: mol_type = other DNA
atcagcgtttacaatggtaacaca

SEQ ID NO: 26 - protein, 8 aa, synthetic construct
  REGION 1..8: note = Synthetic
ISVYNGNT

SEQ ID NO: 27 - DNA, 30 bp, synthetic construct
  misc_feature 1..30: note = Synthetic
  source 1..30: mol_type = other DNA
gcgagagggcaactggactacttcgacccc

SEQ ID NO: 28 - protein, 10 aa, synthetic construct
  REGION 1..10: note = Synthetic
ARGQLDYFDP

SEQ ID NO: 29 - DNA, 318 bp, synthetic construct
  misc_feature 1..318: note = Synthetic
  source 1..318: mol_type = other DNA
gaaattgtgatgacgcagtctccagccaccctgtctgtgtctccaggagaaagagccaccctctcctgcagggccagtcagagtgttagaagcaacttagcctggtaccagcagaaacctggccaggctcccagtctcctcatctatggtgcatccaccagggccactggtatcccagccaggttcagtggcagtgggtctgggacagagttcactctcaccatcagcagcctgcagtctgaagattttgcagtttatttctgtctgcagtataataactggtggacgttcggccaagggaccaaggtggaaatcaaa

SEQ ID NO: 30 - protein, 106 aa, synthetic construct
  REGION 1..106: note = Synthetic
EIVMTQSPATLSVSPGERATLSCRASQSVRSNLAWYQQKPGQAPSLLIYGASTRATGIPARFSGSGSGTEFTLTISSLQSEDFAVYFCLQYNNWWTFGQGTKVEIK

SEQ ID NO: 31 - DNA, 18 bp, synthetic construct
  misc_feature 1..18: note = Synthetic
  source 1..18: mol_type = other DNA
cagagtgttagaagcaac

SEQ ID NO: 32 - protein, 6 aa, synthetic construct
  REGION 1..6: note = Synthetic
QSVRSN

SEQ ID NO: 33-34 - skipped (2 entries)

SEQ ID NO: 35 - DNA, 24 bp, synthetic construct
  misc_feature 1..24: note = Synthetic
  source 1..24: mol_type = other DNA
ctgcagtataataactggtggacg

SEQ ID NO: 36 - protein, 8 aa, synthetic construct
  REGION 1..8: note = Synthetic
LQYNNWWT

SEQ ID NO: 37 - DNA, 1335 bp, synthetic construct
  misc_feature 1..1335: note = Synthetic
  source 1..1335: mol_type = other DNA
caggttcagctggtgcagtctggagctgaggtgaagaagcctggggcctcagtgagggtctcctgcaaggcttctggttacacctttaccaattatggtatcacctgggtgcgacaggcccctggacaagggcttgagtggatgggagggatcagcgtttacaatggtaacacagaatatgcagagaaattccagggcagagtctccatgaccacagacacatccacgaccacagcctacttggagctgaggagcctgaaatctgacgacacggccgtctatttctgtgcgagagggcaactggactacttcgacccctggggccagggaaccctggtcactgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 444 aa, synthetic construct
  REGION 1..444: note = Synthetic
QVQLVQSGAEVKKPGASVRVSCKASGYTFTNYGITWVRQAPGQGLEWMGGISVYNGNTEYAEKFQGRVSMTTDTSTTTAYLELRSLKSDDTAVYFCARGQLDYFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 642 bp, synthetic construct
  misc_feature 1..642: note = Synthetic
  source 1..642: mol_type = other DNA
gaaattgtgatgacgcagtctccagccaccctgtctgtgtctccaggagaaagagccaccctctcctgcagggccagtcagagtgttagaagcaacttagcctggtaccagcagaaacctggccaggctcccagtctcctcatctatggtgcatccaccagggccactggtatcccagccaggttcagtggcagtgggtctgggacagagttcactctcaccatcagcagcctgcagtctgaagattttgcagtttatttctgtctgcagtataataactggtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 213 aa, synthetic construct
  REGION 1..213: note = Synthetic
EIVMTQSPATLSVSPGERATLSCRASQSVRSNLAWYQQKPGQAPSLLIYGASTRATGIPARFSGSGSGTEFTLTISSLQSEDFAVYFCLQYNNWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 363 bp, synthetic construct
  misc_feature 1..363: note = Synthetic
  source 1..363: mol_type = other DNA
cagctgcagctgcaggagtcgggcccaggactggtgaagccttcggagaccctgtccctcacctgcactgtctctggtggctccatcaacagtaatagttactactggggctggatccgccagcccccagggaagggactggagtggattgggagtatctattatgataggaacacctactacaccccgtccctcaagagtcgagtcaccatatccgtagacacgtccaagaaccagttctccctgaagctgacctctgtgaccgccgcagacacggctgtatattattgtgcgagagagcaggtccccggctactacggtatggacgtctggggccaagggaccacggtcaccgtctcctca

SEQ ID NO: 42 - protein, 121 aa, synthetic construct
  REGION 1..121: note = Synthetic
QLQLQESGPGLVKPSETLSLTCTVSGGSINSNSYYWGWIRQPPGKGLEWIGSIYYDRNTYYTPSLKSRVTISVDTSKNQFSLKLTSVTAADTAVYYCAREQVPGYYGMDVWGQGTTVTVSS

SEQ ID NO: 43 - DNA, 30 bp, synthetic construct
  misc_feature 1..30: note = Synthetic
  source 1..30: mol_type = other DNA
ggtggctccatcaacagtaatagttactac

SEQ ID NO: 44 - protein, 10 aa, synthetic construct
  REGION 1..10: note = Synthetic
GGSINSNSYY

SEQ ID NO: 45 - DNA, 21 bp, synthetic construct
  misc_feature 1..21: note = Synthetic
  source 1..21: mol_type = other DNA
atctattatgataggaacacc

SEQ ID NO: 46 - protein, 7 aa, synthetic construct
  REGION 1..7: note = Synthetic
IYYDRNT

SEQ ID NO: 47 - DNA, 39 bp, synthetic construct
  misc_feature 1..39: note = Synthetic
  source 1..39: mol_type = other DNA
gcgagagagcaggtccccggctactacggtatggacgtc

SEQ ID NO: 48 - protein, 13 aa, synthetic construct
  REGION 1..13: note = Synthetic
AREQVPGYYGMDV

SEQ ID NO: 49 - DNA, 321 bp, synthetic construct
  misc_feature 1..321: note = Synthetic
  source 1..321: mol_type = other DNA
gacatccagttgacccagtctccatccttcctgtctgcatctgtgggagacagagtcaccatcacttgctgggccagtcagggcattagcagatatttagcctggtatcagcaaaaaccagggaaaacccctaaactcctgatctatgctgcatccactttgcaaagtggggtcccatcaaggttcagcggcactggatctgggacagatttcactctcacaatcagcaacctgcagcctgaagattttgcaacttattactgtcaacagtttgatagttacccgtggacgttcggccaagggaccaaggtggaaatcaaa

SEQ ID NO: 50 - protein, 107 aa, synthetic construct
  REGION 1..107: note = Synthetic
DIQLTQSPSFLSASVGDRVTITCWASQGISRYLAWYQQKPGKTPKLLIYAASTLQSGVPSRFSGTGSGTDFTLTISNLQPEDFATYYCQQFDSYPWTFGQGTKVEIK

SEQ ID NO: 51 - DNA, 18 bp, synthetic construct
  misc_feature 1..18: note = Synthetic
  source 1..18: mol_type = other DNA
cagggcattagcagatat

SEQ ID NO: 52 - protein, 6 aa, synthetic construct
  REGION 1..6: note = Synthetic
QGISRY

SEQ ID NO: 53-54 - skipped (2 entries)

SEQ ID NO: 55 - DNA, 27 bp, synthetic construct
  misc_feature 1..27: note = Synthetic
  source 1..27: mol_type = other DNA
caacagtttgatagttacccgtggacg

SEQ ID NO: 56 - protein, 9 aa, synthetic construct
  REGION 1..9: note = Synthetic
QQFDSYPWT

SEQ ID NO: 57 - DNA, 1347 bp, synthetic construct
  misc_feature 1..1347: note = Synthetic
  source 1..1347: mol_type = other DNA
cagctgcagctgcaggagtcgggcccaggactggtgaagccttcggagaccctgtccctcacctgcactgtctctggtggctccatcaacagtaatagttactactggggctggatccgccagcccccagggaagggactggagtggattgggagtatctattatgataggaacacctactacaccccgtccctcaagagtcgagtcaccatatccgtagacacgtccaagaaccagttctccctgaagctgacctctgtgaccgccgcagacacggctgtatattattgtgcgagagagcaggtccccggctactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 58 - protein, 448 aa, synthetic construct
  REGION 1..448: note = Synthetic
QLQLQESGPGLVKPSETLSLTCTVSGGSINSNSYYWGWIRQPPGKGLEWIGSIYYDRNTYYTPSLKSRVTISVDTSKNQFSLKLTSVTAADTAVYYCAREQVPGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - DNA, 645 bp, synthetic construct
  misc_feature 1..645: note = Synthetic
  source 1..645: mol_type = other DNA
gacatccagttgacccagtctccatccttcctgtctgcatctgtgggagacagagtcaccatcacttgctgggccagtcagggcattagcagatatttagcctggtatcagcaaaaaccagggaaaacccctaaactcctgatctatgctgcatccactttgcaaagtggggtcccatcaaggttcagcggcactggatctgggacagatttcactctcacaatcagcaacctgcagcctgaagattttgcaacttattactgtcaacagtttgatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 214 aa, synthetic construct
  REGION 1..214: note = Synthetic
DIQLTQSPSFLSASVGDRVTITCWASQGISRYLAWYQQKPGKTPKLLIYAASTLQSGVPSRFSGTGSGTDFTLTISNLQPEDFATYYCQQFDSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 1273 aa, synthetic construct
  REGION 1..1273: note = Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - protein, 5 aa, synthetic construct
  REGION 1..5: note = Synthetic
GGGGS

SEQ ID NO: 63 - protein, 492 aa, synthetic construct
  REGION 1..492: note = Synthetic
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQMYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 64 - protein, 492 aa, synthetic construct
  REGION 1..492: note = Synthetic
MALNSGSPPGVGPYYENHGYQPENPYPAQPTVAPNVYEVHPAQYYPSPVPQYTPRVLTHASNPAVCRQPKSPSGTVCTSKTKKALCVTMTLGAVLVGAALAAGLLWKFMGSKCSDSGIECDSSGTCISLSNWCDGVSHCPNGEDENRCVRLYGPNFILQVYSSQRKSWHPVCRDDWNENYARAACRDMGYKNSFYSSQGIVDNSGATSFMKLNTSAGNVDIYKKLYHSDACSSKAVVSLRCIACGVRSNLSRQSRIVGGQNALLGAWPWQVSLHVQNIHVCGGSIITPEWIVTAAHCVEKPLNSPWQWTAFVGTLRQSSMFYEKGHRVEKVISHPNYDSKTKNNDIALMKLHTPLTFNEVVKPVCLPNPGMMLEPEQHCWISGWGATQEKGKTSDVLNAAMVPLIEPRRCNNKYVYDGLITPAMICAGFLQGTVDSCQGDSGGPLVTLKNDVWWLIGDTSWGSGCAQANRPGVYGNVTVFTDWIYRQMRADG